SEQUENCE LISTING (4 sequences)

SEQ ID NO: 1 - DNA, 20 bp, Homo sapiens
  source 1..20: mol_type = other DNA
gatagtaatcccaacagacc

SEQ ID NO: 2 - DNA, 20 bp, Homo sapiens
  source 1..20: mol_type = other DNA
gacccccaggagtgtgacgg

SEQ ID NO: 3 - DNA, 59 bp, Homo sapiens
  source 1..59: mol_type = other DNA
cgcatgtatttattaccagacgccgccgcctcaggagccgccgccacacacgtgtcaag

SEQ ID NO: 4 - DNA, 60 bp, Homo sapiens
  source 1..60: mol_type = other DNA
ctttgacacgtgtgtggcggcggctcctgaggcggcggcgtctggtaataaatacatgcg

(19) United States
(12) Patent Application Publication
(10) Pub. No.: US 2023/0263797 A1
(43) Pub. Date: Aug. 24, 2023

(54) Mammalian Hybrid Pre-Autophagosomal Structure HyPAS and Compositions and Methods for the Treatment of Coronavirus Infections and Related Disease States

(71) Applicant: UNM Rainforest Innovations, Albuquerque, NM (US)

(72) Inventors: Vojo P. Deretic, Placitas, NM (US); Graham Timmins, Albuquerque, NM (US); Suresh Kumar, Albuquerque, NM (US)

(21) Appl. No.: 17/895,849

(22) Filed: Aug. 25, 2022

Related U.S. Application Data
(60) Provisional application No. 63/237,293, filed on Aug. 26, 2021.

(51) Int. Cl. A61K 31/495; A61K 31/03; A61K 31/135; A61P 31/14

(52) U.S. Cl. CPC A61K 31/495; A61K 31/03; A61K 31/135; A61P 31/14

(57) ABSTRACT

The present invention is directed to elucidating the mechanism of formation of autophagosomal membranes that emerge via convergence of secretory and endosomal pathways and providing therapeutic approaches to the treatment of several acute respiratory syndrome coronavirus 1 (SARS) and 2 SARS-CoV-2), MERS-CoV and numerous other autophagy-mediated diseases based upon this mechanism. This process is targeted by microbial factors such as coronaviral membrane modulating proteins and represents a potential target for agents which may be used in the treatment of SARS, COVID and MERS. The present invention is also directed to compositions and methods for the treatment of SARS, COVID and/or MERS and other autophagy-mediated disease states.

### RELATED APPLICATIONS AND GRANT SUPPORT

This application claims the benefit of priority of U.S. provisional application Ser. No. 63/237,293, filed Aug. 26, 2021, the entire contents of which is incorporated by reference herein.

This invention was made with government support under grant nos. R37AI042999 and R01AI111935 awarded by the National Institutes of Health. The government has certain rights in the invention.

### FIELD OF THE INVENTION

The present invention is directed to elucidating the mechanism of formation of autophagosomal membranes that emerge via convergence of secretory and endosomal pathways and providing therapeutic approaches to the treatment of several acute respiratory syndrome coronavirus 1 (SARS-CoV-1), 2 (SARS-CoV-2) or MERS-CoV and related disease states and/or conditions and numerous other autophagy-mediated diseases based upon this mechanism. This process is targeted by microbial factors such as coronaviral membrane modulating proteins and represents a potential target for agents which may be used in the treatment of SARS, COVID 19 and MERS, among other disease states and/or conditions. The present invention is also directed to compositions and methods for the treatment of SARS, COVID19 and/or MERS and other autophagy-mediated disease states.

### INCORPORATION BY REFERENCE

This application incorporates the Sequence Listing titled “Sequence_Listing_N12-355US”, created on May 11, 2023, and has a file size of 4,722 bytes by reference in its entirety herein.

### BACKGROUND AND OVERVIEW OF THE INVENTION

The novel SARS-CoV-like virus, SARS-CoV-2, related to SARS-CoV (SARS) but a distinct form of it, was identified as the causative agent of the recent 2019-2020 outbreak of viral pneumonia that started in Wuhan, China, which spread to all permanently inhabited continents. The human-to-human spread of CoV2 has resulted in a runaway global pandemic, causing a disease termed COVID-19 (coronavirus disease-2019) by the World Health Organization. The COVID-19 pandemic escalated at an alarming rate with millions infected, as of this writing, and tens of thousands of deaths just in Italy and Spain with a high mortality rate in these disproportionately affected countries away from the epicenter of the initial epidemic. COVID-19 and its variants threaten worldwide populations based on its pernicious combination of long incubation times coupled with exceptional spreading potential, and significantly high morbidity and mortality. The reproductive number R₀ initially estimated to be 2.2-2.7 according to some estimates may be as high as 4.7-6.6. There is an urgent public health need to understand SARS-CoV-2 as a virus, COVID-19 as a disease, and COVID-19 as an epidemiological phenomenon. The development of countermeasures, including therapeutics aiming to lessen disease severity and to come up with prophylaxes including vaccines is occurring at a rapid rate.

Recently, FDA approved drugs, chloroquine (CQ) and hydroxychloroquine (HCQ) and azithromycin (AZT) have shown therapeutic effects in in COVID-19, using viral loads as endpoints in initial clinical trials and also in in vitro cellular systems. Both HCQ and CQ are known inhibitors of autophagy, and so the field is actively looking to repurpose and develop other inhibitors of autophagy that may also show such activity.

Autophagy is a fundamental biological process contributing to cytoplasmic quality control and cellular metabolism (Deretic and Kroemer, 2021; Levine and Kroemer, 2019; Mizushima et al., 2011) with implications in cancer, infection, metabolic disorders, aging, and neurodegeneration (Deretic, 2021; Klionsky et al., 2021; Levine and Kroemer, 2019). The mammalian autophagy pathway induced by starvation (Morishita and Mizushima, 2019) is controlled by several protein modules (Morishita and Mizushima, 2019). This includes the FIP200 complex with ULK1/2 kinase acting as the conduit for regulation by mTOR and AMPK, and a protein lipidation system which includes ATG16L1 and results in membrane association of mammalian Atg8 proteins (mAtg8s), such as the autophagosomal marker LC3B (Morishita and Mizushima, 2019). Autophagosomes are presumed to originate from pre-existing membranes contributed by a number of putative sources (Melia et al., 2020). Through subsequent stages, mammalian autophagosomes enlarge, envelop cargo, and merge with lysosomes (Itakura et al., 2012; Matsui et al., 2018b; Reggiori and Ungermann, 2017) whereby the cargo is degraded.

Mammalian systems controlling autophagy include less understood but important contributors, extended synaptotagmins (E-SYTs) (Nascimbeni et al., 2017), and sigma receptor 1 (SIGMAR1) (Yang et al., 2019). E-SYTs function as Ca²⁺-regulated tethers between endoplasmic reticulum (ER) and plasma membrane (PM) and participate in intermembrane lipid transfer (Saheki et al., 2016). They localize to ER (Saheki et al., 2016; Sclip et al., 2016), PM (Giordano et al., 2013; Min et al., 2007) and membranes with ATG16L1 and LC3 (Nascimbeni et al., 2017). E-SYT2 affects autophagosome biogenesis by regulating phosphatidylinositol-3-phosphate (PI3P) synthesis at the ER-PM contact sites (Nascimbeni et al., 2017). SIGMAR1, an ER resident protein and Ca²⁺-regulator participating in autophagy (Christ et al., 2019; Vollrath et al., 2014; Yang et al., 2019), is a trimeric transmembrane ER protein (Schmidt et al., 2016) with many physiological effects, including Ca²⁺ transactions at ER-mitochondria contacts (Hayashi and Su, 2007). SIGMAR1 has roles in cancer (Vilner et al., 1995) and neurodegeneration including Alzheimer's disease (Feher et al., 2012) and amyotrophic lateral sclerosis (Vollrath et al., 2014; Watanabe et al., 2016). SIGMAR1 interacts with SNAREs implicated in autophagy, STX17 and VAMP8, and with ATG14L (Yang et al., 2019), a component of PI3KC1 involved in autophagy initiation (Baskaran et al., 2014; Chang et al., 2019). SIGMAR1 interacts with SARS-CoV-2 nsp6 (Gordon et al., 2020) and is a target for pharmacological agents such as chloroquine (CQ) (Gordon et al., 2020; Hirata et al., 2011; Schmidt et al., 2016).

A key area of interest in autophagy research is the integration of known protein complexes with the provenance and source of membranes for autophagosome formation and growth (Hamasaki et al., 2013; Lamb et al., 2013; Melia et al., 2020; Moreau et al., 2011; Nishimura et al., 2017). Here we show that FIP200 and ATG16L1 reside on two distinct sources of membranes, cis-Golgi and PM-derived endosomes respectively, which merge to form autophagosomes. This is a pivotal event leading to the formation of hybrid pre-autophagosomal structures (HyPAS) during autophagosomal biogenesis. Further, HyPAS is a hitherto unknown cellular target perturbed by SARS-CoV-2, positioned at the crossroads between autophagy and the biogenesis of specialized coronavirus-induced compartments (Cottam et al., 2011; Cottam et al., 2014; Fung and Liu, 2019; Gassen et al., 2019; Ghosh et al., 2020; Reggiori et al., 2010).

### BRIEF DESCRIPTION OF THE INVENTION

In an embodiment, the present invention is directed to the use of a modulator of the SIGMA1 receptor (“SIGMA1 receptor modulator”), especially Cutamesine, BD-1047 or an isotopomer or mixture thereof in the treatment of SARS, SARS-CoV-2 and/or MERS or other autophagy mediated disease state and/or condition. In embodiments, the SIGMA1 receptor modulator is combined with an additional agent such as ambroxol, bromhexine, chloroquine, hydroxychloroquine, bafilomycin A1 (BafA1), azithromycin, zinc or a mixture thereof or an additional autophagy modulator for the treatment of SARS, COVID19/SARSCoV-2 and/or MERS or other autophagy mediated disease state and/or condition. In an embodiment, one or more of these SIGMA1 receptor modulators, especially at least one isotopomer of Cutamesine or BD-1047 is combined with a pharmaceutically acceptable carrier, additive or excipient in pharmaceutical dosage form. In embodiments, the SIGMA1 receptor modulator is combined with at least one compound selected from the group consisting of ambroxol, bromhexine, chloroquine, hydroxychloroquine, bafilomycin A1 (BafA1), azithromycin, zinc, molnupiravir, baricitinib, nirmatrelvir and ritonavir (Paxlovid), or a mixture thereof or an additional autophagy modulator as described in greater detail herein in a one or more part compositions often in combination with a carrier, additive or excipient. This composition is administered to a patient in need to treat a coronavirus infection, in particular a SARS, SARS-CoV-2 (Covid 19) or MERS coronavirus infection or other autophagy mediated disease state or conditions. In an embodiment, the ambroxol, bromhexine, chloroquine (CQ), hydroxychloroquine (HCQ), bafilomycin A1 (BafA1), ivermectin, azithromycin, zinc, olnupiravir, baricitinib, nirmatrelvir and ritonavir (Paxlovid) or a mixture thereof or an additional autophagy agent is included in the pharmaceutical dosage form with the SIGMA1 receptor modulator, especially cutamesine, BD-1047, an isotopomer thereof or a mixture thereof or is separately co-administered alone along with the SIGMA1 receptor modulator, especially cutamesine, BD-1047 or an isotopomer thereof. Co-administration provides a particularly potent effect, including a synergistic inhibition of SARS-CoV-1, SARS-CoV-2 or MERS-CoV. In other embodiments, the present invention is directed to methods of treating coronavirus, including SARS-CoV-1, SARS-CoV-2 (Covid-19) or MERS-CoV where effective amounts of the agent(s) as set forth above are administered to a patient in need to provide a favorable impact on the outcome of therapy of the viral infection.

In embodiments, the invention is directed to treating an autophagy infection with at least one SIGMA1 receptor modulator (e.g. agonist and/or antagonist), especially cutamesine, BD-1047 or an isotopomer thereof optionally in combination with an additional autophagy modulator, especially including ambroxol and/or bromhexine.

In embodiments, the present invention is directed to a method of treating a SARS-CoV-1, SARS-CoV-2 or MERS-CoV infection, acute respiratory distress syndrome (ARDS), arrhythmia, long-term reduction in lung function and/or disability secondary to a coronavirus (especially Covid 19) infection in a subject in need. This method comprises administering to said subject in need an effective amount of a modulator of SIGMA1 receptor, especially at least one of cutamesine, BD-1047 or an isotopomer thereof optionally in combination with chloroquine, hydroxychloroquine, bafilomycin A1 (BafA1), ivermectin, azithromycin, zinc, molnupiravir, baricitinib, nirmatrelvir and ritonavir (Paxlovid) or a mixture thereof or an additional autophagy modulator for the treatment of SARS, COVID19 and/or MERS or other autophagy mediated disease state or condition.

In embodiments, the present invention is directed to pharmaceutical compositions comprising an effective amount of a modulator of SIGMA1 receptor, especially at least one compound selected from the group consisting of haloperidol, cutamesine, an isotopomer of cutamesine, BD-1047, an isotopomer of BD-1047 or a mixture thereof, optionally in combination with an effective amount of at least one additional agent selected from the group consisting of ambroxol, bromhexine, chloroquine (CQ), hydroxychloroquine (HCQ), bafilomycin A1 (BafA1), ivermectin, azithromycin, molnupiravir, baricitinib, nirmatrelvir and ritonavir (Paxlovid) and zinc (preferably HCQ, azithromycin and/or zinc) or an additional autophagy modulator in order to provide a particularly favorable impact on Covid-19 therapy in pharmaceutical dosage form for administration to a patient. In certain embodiments, the pharmaceutical composition comprises a mixture of cutamesine, BD-1047 or an isotopomer thereof. In embodiments, the pharmaceutical composition comprises cutamesine and BD-1047. In embodiments, the pharmaceutical composition comprises an isotopomer of cutamesine, BD-1047 or a mixture thereof.

In embodiments, a modulator of SIGMA 1 receptor as disclosed herein may be used to treat a number of other autophagy mediated disease states and/or conditions. A SIGMA 1 receptor antagonist, especially BD-1047 or an isotopomer thereof finds use in the treatment of cancer, rheumatoid arthritis, malaria, antiphospholipid antibody syndrome, lupus, chronic urticaria or Sjogren's disease, among others. A SIGMA 1 receptor agonist finds use in treating a number of autophagy mediated disease states and/or conditions as described herein.

These and/or other embodiments of the present invention may be readily gleaned from a review of the detailed description of the invention which follows.

### BRIEF DESCRIPTION OF THE FIGURES

FIG. 1 shows that STX17 is required for colocalization between FIP200 and ATG16L1. (A) Effects of autophagy induction (EBSS 2 h) on colocalization (confocal microscopy) between endogenous FIP200 and ATG16L1 in HeLa cells. Scale bar, 5 μm. (B) HCM quantification of colocalization between endogenous FIP200 and ATG16L1 in HeLa cells. (C) HCM quantification of colocalization between FIP200 and FLAG-ATG16L1 in HeLa cells. B and C, **, p<0.01, (n=3) t-test. (D) (i-iii) CLEM of HeLa cells expressing GFP-FIP200 and mCherry-ATG16L1. Boxed area (panel i) is shown at TEM level in panels ii-iii, representing two Z sections 70 nm apart. Panel ii′, overlay of fluorescence and TEM. Yellow fluorescence in panel a corresponds to vesicular (white arrows) and tubular (gray arrows) elements in TEM. Asterisk, a small double-membrane structure or a cross-section of a cup-shaped cisternal element. White asterisks (panels (v and v′) correspond to phagophore membrane. Black asterisk (panel vi), ER-mitochondrial contact site. The membranes of the phagophore (red) and ER surrounding it (white) are traced in panel vii. Nu, nucleus, Mi, mitochondrion. Scale bar in (i) and (iv) 5 μm. (E) Effect of STX17 KO on colocalization (confocal microscopy) between endogenous FIP200 and FLAG-ATG16L1 in HeLa cells induced for autophagy (EBSS, 2 h). Scale bar, 5 μm. (F,G) STX17 KO effects on colocalization between FIP200 and FLAG-ATG16L1 in HeLa and Huh7 cells (HCM). **, p<0.01, (n=3) ANOVA. (H,I) Super-resolution microscopy and cluster analysis of FLAG-ATG16L1 and endogenous FIP200 in HeLa^{WT} or STX17^{KO} cells incubated with EBSS for 2 h. See also Figures S1 and S2.

FIG. 2 shows that STX17 is required for HyPAS formation. (A) Schematic of in vitro fusion assay (ivitHC) between GFP-FIP200 and mCherry-ATG16L1 compartments utilizing high content microscopy quantification. (B,C) Quantification of the effects of STX17 KO on HyPAS formation in vitro (IvitHC). Scale bar 500 nm. (D) PBMM assay (schematic) is a combination of RUSH and APEX-2 proximity biotinylation. (E-H) PBMM quantification of the effects of STX17 KO and FIP200 KO on starvation-induced mixing of early/cis-Golgi (ManII) and endosomal (TFRC) compartments. † p≥0.05, **, p<0.01, (n=3) ANOVA. See also Figures S2 and S3.

FIG. 3 shows that STX17 is required for association between FIP200 and ATG16L1. (A,B) Co-IP analysis and quantifications of FIP200 and FLAG-ATG16L1 interactions in WT or STX17^{KO} HeLa cells. **, p<0.01, (n=3) t-test. (C) MS analysis, GFP vs. GFP-STX17 peptides in VAMP7 complexes. (D,E) Co-IP analysis of interactions between endogenous proteins: VAMP7 or STX17, with ATG16L1 or FIP200. (F) Effects of VAMP7 KD on colocalization between FIP200 and FLAG-ATG16L1 in HeLa cells (HCM quantification). Y-axis, % of FIP200⁺ profiles that were also ATG16L1⁺ (a measure of HyPAS). **, p<0.01, (n=3) ANOVA. (G) Effect of VAMP7 KD on HyPAS formation (confocal microscopy) in HeLa cells induced for autophagy (EBSS, 2 h). Scale bar, 5 μm. Individual channels related to this panel are shown in Figure S3U. (H-J) ivitHC schematic, quantification, and images of VAMP7 KD effect on HyPAS formation in vitro. Vesicle source cells were induced for autophagy by incubating in EBSS for 2 h. † p≥0.05, **, p<0.01, (n=3) ANOVA. Scale bar 500 nm. (K) Effects of TBK1 KO on HyPAS formation (confocal microscopy) in cells induced for autophagy (EBSS; 2 h). Scale bar, 5 μm. (L) HCM quantification of the effects of TBK1 KO on HyPAS formation. **, p<0.01, (n=3) ANOVA. (M) Complementation analysis (HyPAS quantification by HCM): STX17 KO cells transfected with GFP fusions with STX17 wild type, non-phosphorylatable mutant of STX17 (S202A) or phosphomimetic mutant of STX17 (S202D). † p≥0.05, **, p<0.01, (n=3) ANOVA. See also Figures S3 and S4.

FIG. 4 shows that Ca²⁺ positively regulates HyPAS formation. (A) SERCA2 peptides (MS analysis) in GFP or GFP-STX17 complexes. (B) Co-IP analysis with GFP-STX17 and endogenous SERCA2. (C,D) Effects (C, HCM; D, confocal microscopy) of BAPTA-AM treatment on HyPAS formation in WT and STX17^{KO} HeLa cells, **, p<0.01, (n=3) ANOVA. Scale bar, 5 μm. (E,F) Effect of BAPTA on HyPAS formation in vitro (IvitHC assay) in cells induced for autophagy. **, p<0.01, (n=3) ANOVA. Scale bar, 1 μm. (G) Effect of thapsigargin treatment on HyPAS formation in WT and STX17^{KO} HeLa cells (HCM). **, p<0.01, (n=3) ANOVA. (H) Confocal microscopy images of the effect of thapsigargin treatment on HyPAS formation in WT and STX17^{KO} HeLa cells. Scale bar, 5 μm. (I) Confocal microscopy analysis of colocalization between FLAG-SERCA2, FIP200 and mCherry-ATG16L1 in HeLa cells induced for autophagy (EBSS, 2 h). Scale bar, 5 μm. (J,K) Flow cytometry analysis of the effect of STX17 KO on FLUO-3AM fluorescence in cells incubated in full media or in HBSS for 4 h. **, p<0.01, (n=3) ANOVA. (L) A schematic representation of the effect of STX17 on SERCA2 and Ca²⁺ flow from cytosol to ER lumen. See also Figure S4.

FIG. 5 shows that STX17 interactor E-SYT2 controls HyPAS formation. (A) E-SYT2 peptides (MS analysis) in GFP or GFP-STX17 complexes. (B) Co-IP analysis, endogenous STX17 and E-SYT2. (C) Localization (confocal microscopy) of GFP-E-SYT2 and FLAG-STX17 in HeLa cells. Scale bar, 5 μm. (D) Effect of E-SYT2 KO on HyPAS formation (confocal microscopy) in HeLa induced for autophagy (EBSS, 2 h). Scale bar, 5 μm. (E,F) HCM quantification of HyPAS formation in WT or E-SYT2^{KO} HeLa. Masks: white, primary objects (FLAG positive cells), yellow puncta, overlap between FIP200 and FLAG-ATG16L1. **, p<0.01, (n=3) ANOVA. Scale bar, 10 μm. (G,H) In vitro HyPAS formation, effect of E-SYT2 KO (ivitHC quantification). Fusing vesicle preparations were from cells induced for autophagy (EBSS for 2 h). † p≥0.05, **, p<0.01, (n=3) ANOVA. (IJ) PBMM assay and quantifications of the effects of E-SYT2 KO on starvation-induced membrane mixing. The experimental conditions were same as in FIG. 2E,F. † p≥0.05, **, p<0.01, (n=3) ANOVA. (K) Alignment of conserved residues in the polybasic regions of Synaptotagmin 1 and E-SYT2. Upward arrows, key residues in SNARE interactions; downward arrows, basic residues mutated to Ala in E-SYT2^{6A}. (L,M) Co-IP analysis, GFP-E-SYT2 WT or GFP-E-SYT2^{6A} with FLAG-STX17. p>0.05; **p<0.01, (n=3) t-test. (N) Complementation of E-SYT2 KO with WT or E-SYT2^{6A}; HCM analysis of HyPAS formation, † p≥0.05, **, p<0.01, (n=3) ANOVA. (O,P) Co-IP, STX17 and VAMP7 in WT or E-SYT2^{KO} HeLa. **, p<0.01, (n=3) t-test. (Q) Co-IP, FLAG-STX17 and GFP-VAMP7 in WT or E-SYT2^{KO} HeLa. (R) Co-IP, FLAG-STX17 and GFP-VAMP8 in WT or E-SYT2^{KO} HeLa. See also Figure S5.

FIG. 6 shows that SIGMAR1 is required for HyPAS formation and HyPAS apparatus affects autophagy of diverse cargo. (AB) Effect (HCM quantification) of SIGMAR1 KO on HyPAS formation in 293A cells. Masks: white, primary objects (FLAG positive cells), yellow puncta, overlap between FIP200 and FLAG-ATG16L1. Scale bar, 5 μm. (C) Confocal microscopy of HyPAS in WT or SIGMAR1^{KO} 293A cells. Scale bar, 5 μm. (D) Effect of complementation of SIGMAR1 KO with empty FLAG, SIGMAR1-FLAG or with SIGMAR1 N80-FLAG. on HyPAS formation in SIGMAR1^{KO} 293A cells. HCM quantification. (E) Confocal microscopy images related to D. Scale bar, 5 μm. (F,G) Effect of E-SYT2 KO on mitophagy induced by OA (oligomycin+antimycin) treatment. Cells were transfected with YFP-Parkin. YFP-Parkin positive cells were gated for HCM quantification of mito-DNA dots (mitophagy). Masks: white, primary objects (YFP-Parkin positive cells), red puncta, mito-DNA dots. (H) Complementation of E-SYT2 KO with WT or E-SYT2^{6A} in mitophagy assayed by mtDNA clearance (HCM quantifications) in mCherry-Parkin⁺ cells. Masks: white, primary objects (GFP and mCherry positive cells), blue puncta, mtDNA dots. (I) Effect of SIGMAR1 KO on ribophagy using RPL28-Keima probe (HCM quantification). (J,K) Effect of E-SYT2 KO or SIGMAR1 KO on bulk autophagy using LDH-Keima probe (HCM quantification). All graphs, †, p>0.05; **, p<0.01, (n=3) ANOVA. See also Figure S6.

FIG. 7 shows that HyPAS is inhibited in cells infected with SARS-CoV-2 and SARS-CoV-2 nsp6 inhibits HyPAS. (A,B) Effects of SARS-CoV-2 infection (MOI: 1, 24 h) on HyPAS formation in Calu-3 cells; HCM quantification. Masks: white, primary objects (FLAG positive cells), yellow puncta, HyPAS. Scale bar, 10 μm. (C,D) Proximity biotinylation proteomics using 293T-APEX2-nsp6Tet^{ON} cells. Table, subset of SARS-CoV-2-nsp6 interactors; full proteomics data in Table S1. (E-G) Effects (HCM quantification) on HyPAS formation of GFP-nsp6, mChery-ORF3a and mCherry-ORF8 expression. (H-J) Quantification (ivitHC) of the effects of FLAG-nsp6 on HyPAS formation in the vitro fusion assay. Source cells were induced for autophagy (EBSS for 2 h). Scale bar, 0.5 μm. All graphs, † p≥0.05, **, p<0.01, (n=3) ANOVA. (K) Schematic, HyPAS prophagophore formation and the canonical autophagy pathway. HyPAS is generated through a regulated fusion of vesicles and cisternae derived from the early secretory and endosomal pathways to form a prophagophore, which progresses to an LC3-positive phagophore closing to sequester cargo (C) destined for degradation. SARS-CoV-2 infection and at least one of SARS-CoV-2 proteins, nsp6, inhibit HyPAS formation. See also Figure S7.

FIG. 8 shows the chemical synthesis of isotopomeric forms of BD-1047 through deuteration steps using deuterated methyl iodide to introduce deuterated methyl groups on the amine groups. The use of Pd/C catalyst, aluminum catalyst and deuterated water in the presence of microwave radiation deuterates all of the protons in the alkylene chains and the N-alkyl groups.

FIG. 9 shows the chemical synthesis of isotopomeric forms of cutamesine through deuteration steps as presented. The same conditions used for deuteration of BD-1047 presented in the FIG. 8 scheme can provide for the three separate isotopomeric forms of cutamesine. Deuterated methyl iodide is used to introduce deuterated alkoxy groups on the phenyl ring of cutamesine and the use of Pd/C catalyst, aluminum catalyst and deuterated water in the presence of microwave radiation deuterates all of the protons in the alkylene chains and the pyrazine group and the methyl groups on the phenylalkoxy groups when present.

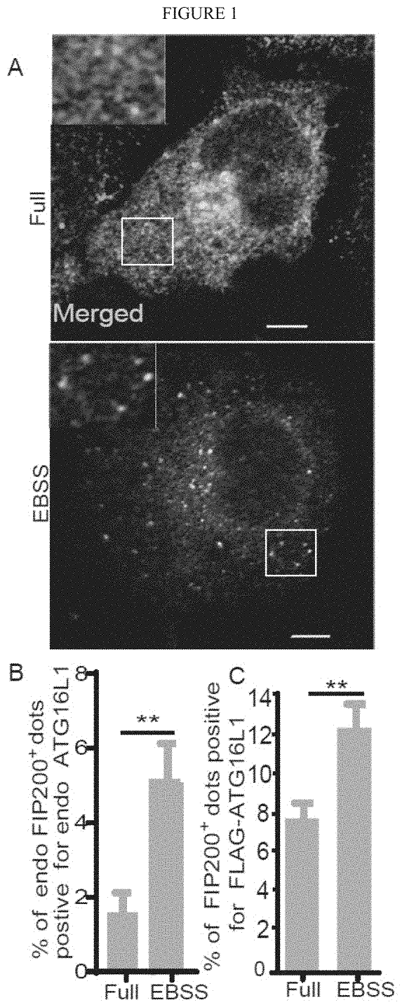

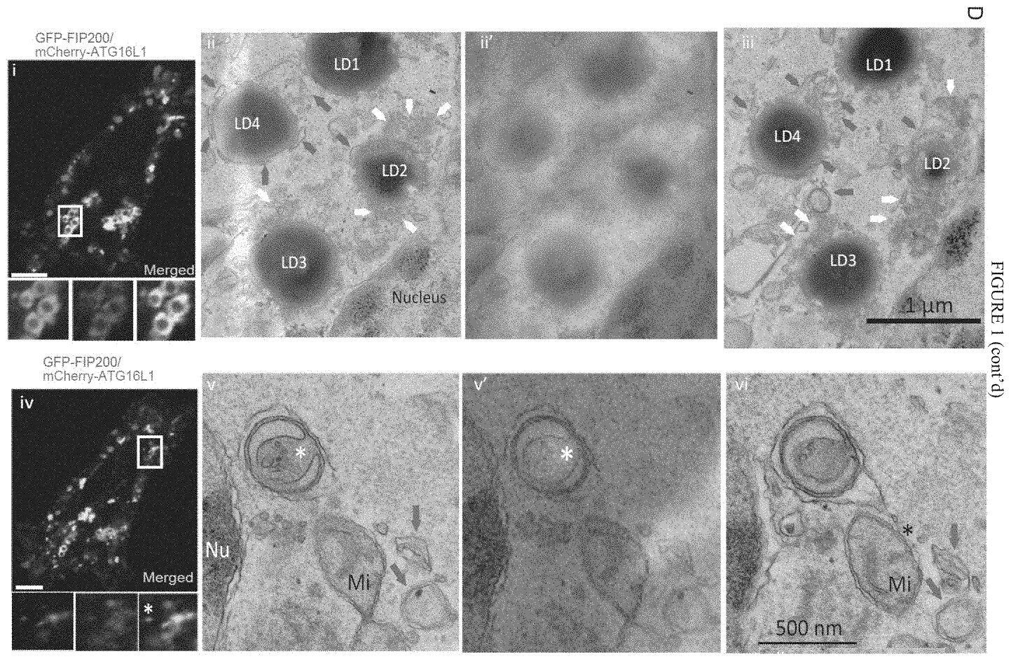

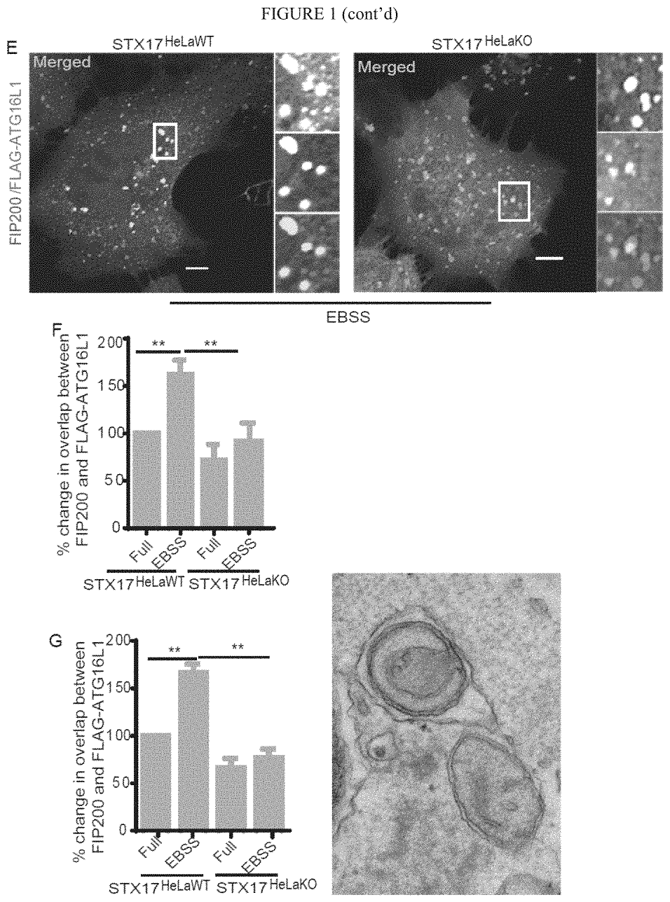

FIGURE S1 shows that FIP200 is localized to cis-Golgi, related to FIG. 1. (A) Effects of autophagy induction (EBSS 2 h) on colocalization (confocal microscopy) between endogenous FIP200 and FLAG-ATG16L1 in HeLa cells. Scale bar, 5 μm. (B,C) HCM quantification (B) and confocal microscopy images (C) of the effect of starvation on colocalization between endogenous FIP200 and FLAG-ATG16L1 in primary normal human bronchial epithelial cells (NHBE) cells. Scale bar, 5 μm. (D) Colocalization (confocal microscopy) of FIP200 with GM130. HeLa; scale bar, 5 μm. (E,F) HCM quantification of % overlap between FIP200 and GM130 in fed (full) or starved (EBSS) HeLa. Masks: white, primary objects (cells), yellow, overlap between FIP200 and GM130. (G-I) Quantification (HCM) of overlap between FIP200 and GM130 as a function of cellular location and number of dispersed punctate FIP200. HeLa, fed vs. starved. (J,K) HCM analysis of the effect of starvation on FIP200 puncta formation in HeLa cells. Masks: white, primary objects (cells), red, FIP200 puncta. HCM, minimum number of wells 6; 3 independent experiments. † p≥0.05, **, p<0.01, (n=3) t-test. (L,M) Effects (HCM) of starvation on FLAG-ATG16L1 puncta formation in HeLa cells. Masks: white, primary objects (cells), red, FIP200 puncta. HCM data in B, F-H, K, L, **, † p≥0.05, p<0.01, (n=3) t-test. Scale bars (E,I,J,L), 10 μm. (N) Confocal microscopy, colocalization between FLAG-ATG16L1, FIP200 and GM130. Scale bar, 5 μm. (O) HCM images of the effect of starvation on colocalization between FLAG-ATG16L1 and FIP200 in HeLa cells. Masks: white, primary objects (FLAG positive cells), yellow puncta, overlap between FIP200 and FLAG-ATG16L1. Scale bar, 10 μm. (P) HCM quantification of the effects of STX17 KO on colocalization between endogenous ATG16L1 and FIP200. **, p<0.01, (n=3) ANOVA. (Q) Localization (confocal microscopy) of endogenous ATG16L1 and FIP200 in HeLa^{WT} or STX17^{KO} cells induced for autophagy by starvation (EBSS). Scale bar, 5 μm. (R,S) HCM quantification of the effect of starvation on colocalization between FLAG-ATG16L1 and FIP200 in Huh7 cells. Masks: white, primary objects (FLAG positive cells), yellow, overlap between FIP200 and FLAG-ATG16L1. **, p<0.01, (n=3) t-test. Scale bar, 10 μm. (T,U) HCM quantification of the effects of starvation on CtxB uptake. **, p<0.01, (n=3) t-test. Scale bar, 10 μm. (V,W) HCM quantification of the effect of starvation on colocalization between FIP200 and Alexa flour-488 cholera toxin B in FLAG-ATG16L1 dots. Masks: white, primary objects (cells), yellow, overlap between FIP200, Alexa flour-488 cholera toxin B and FLAG-ATG16L1 dots. Scale bar, 10 μm. **, p<0.01, (n=3) t-test. (X) Confocal microscopy analysis of colocalization between CtxB, FLAG-ATG16L1 and FIP200 in cells induced for autophagy. Scale bar, 5 μm. (Y,Z) Effects of starvation (HCM) on colocalization between FIP200 and Alexa flour 488 cholera toxin B (CtxB) in HeLa cells. Masks: white, primary objects (cells), yellow, colocalization between FIP200 and CtxB. **, p<0.01, (n=3) t-test. Scale bar, 10 μm. (A¹,B¹) HCM, effect of starvation on colocalization between FLAG-ATG16L1 and CtxB. Masks: white, primary objects (FLAG positive cells), yellow, colocalization between FLAG-ATG16L1 and CtxB. **, p<0.01, (n=3) t-test. In panels T-B¹, for CtxB internalization, cells were incubated with Alexa Fluor-488-conjugated cholera toxin subunit B for 15 min at 4° C. (allowing toxin to bind to the plasma membrane). Then cells were incubated at 37° C. (allowing internalization of cholera toxin) for 10 min and fixed for HCM analysis. Scale bar, 10 μm. (C¹,D¹) HCM, effect of ATG9KO on colocalization between FIP200 and FLAG-ATG16L1 in WT or ATG9^{KO} Huh7 cells incubated in full media or EBSS for 2 h. Masks: white, primary objects (cells), yellow, colocalization between FIP200 and FLAG-ATG16L1. † p≥0.05, **, p<0.01, (n=3) ANOVA. Scale bar, 10 μm. All HCM data, >500 primary objects counted per well; number of wells ≥6/variable/plate; n, number of independent experiments (each on a different plate).

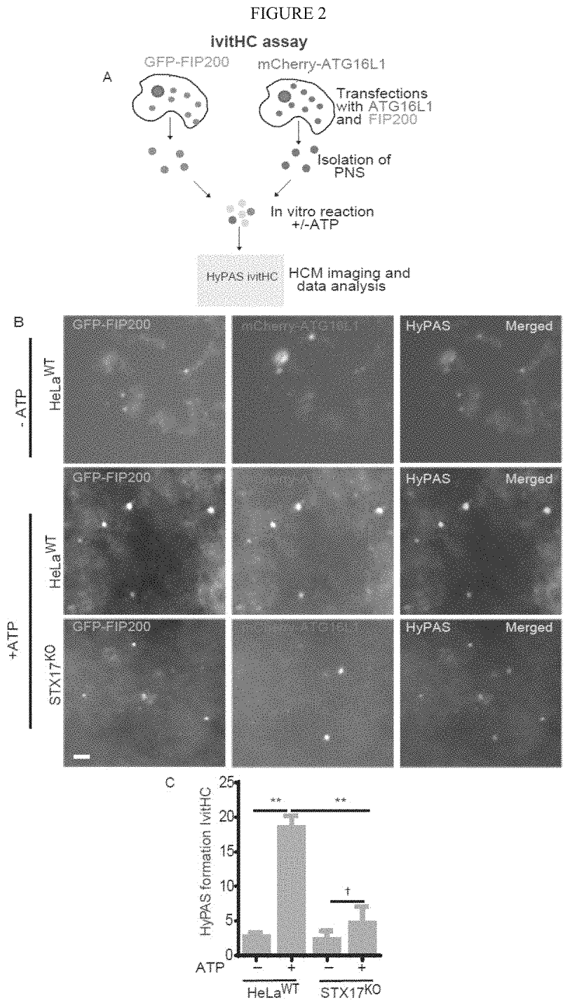

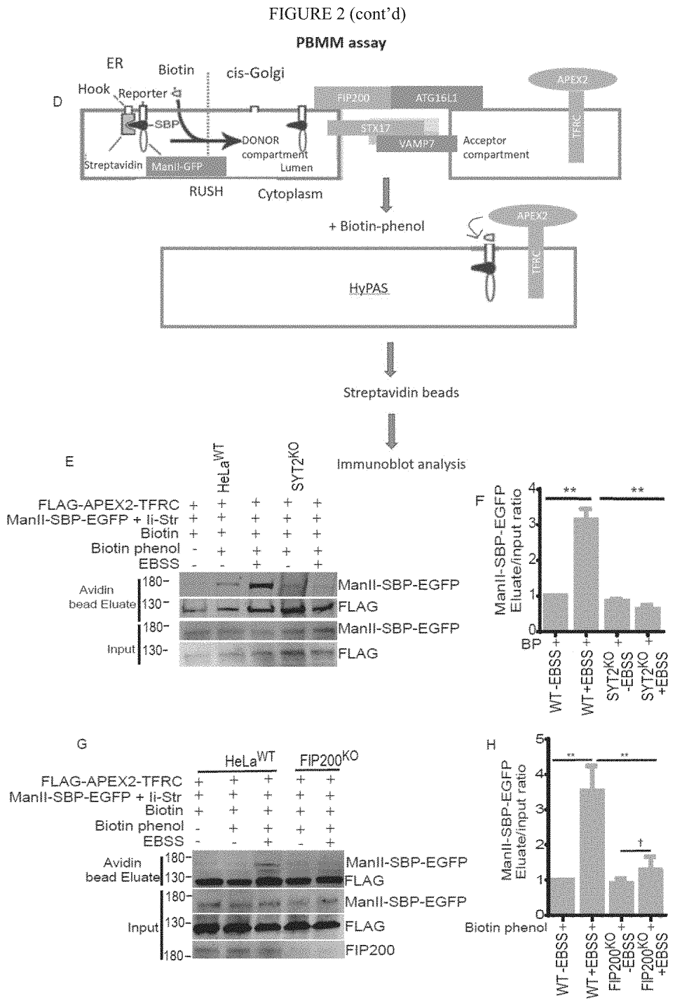

FIGURE S2 shows that endosomal and cis Golgi compartments merge in STX17-dependent and ATG9-independent fashion, related to FIGS. 1 and 2. (A) Confocal images (related to FIG. 1E) showing separate channels. Scale bar, 5 μm. (B) Examples of images and machine imputed masks from a large HCM dataset for quantifying colocalization between FLAG-ATG16L1 and FIP200 in WT vs. STX17^{KO} HeLa cells incubated in full media or in EBSS for 2 h. Masks: white, primary objects (FLAG positive cells), yellow, overlap between FIP200 and FLAG-ATG16L1. Scale bar, 10 μm. (C,D) HCM quantification, effects of rescuing STX17KD in human cells (HeLa) with murine Myc-Stx17 on colocalization between FIP200 and FLAG-ATG16L1. **, p<0.01, (n=3) ANOVA. Scale bar, 10 μm. (E,F) Confocal microscopy and HCM analysis of the effects of starvation on colocalization of GFP-STX17 with FLAG-ATG16L1 and FIP200. Scale bar, 5 μm. HCM, **, p<0.01, (n=3) t-test. (G,H) HCM analysis of the effects of STX17 KO on size of FLAG-ATG16L1 puncta in HeLa cells. HCM, **, p<0.01, (n=3) t-test. Scale bar, 10 μm. (I,J) HCM, effects of STX17 KO on colocalization between TFRC and FLAG-ATG16L1. Scale bar, 10 μm **, p<0.01, (n=3) ANOVA. (K) HCM images, effect of STX17 KO on colocalization between FLAG-ATG16L1 and FIP200 in WT or STX17^{KO} Huh7 cells incubated in full media or in EBSS for 2 h. Scale bar, 10 μm. Masks: white, primary objects (FLAG positive cells), yellow puncta, overlap between FIP200 and FLAG-ATG16L1. (L) IvitHC, HyPAS formation yields in vitro. Source cells were incubated in full media or in EBSS for 15-120 min as indicated. At least 5,000 objects were counted per well and a minimum of 5 wells were used for quantification. † p≥0.05, **p<0.01, (n=3) ANOVA. (M) HCM images from in vitro assay displaying the effects of ATP on in vitro fusion between GFP-FIP200 and mCherry-ATG16L1 in cells induced for autophagy for 60 min. Scale bar, 500 nm. (N) Confocal microscopy (control for the PBMM assay), biotin release of ManII-GFP from the RUSH system hook (Ii in ER; see methods) to translocate to the cis-Golgi (marked by GM130). Scale bar, 5 μm. (O) ManII released from the Ii ER hook by giving biotin to cells colocalizes with FIP200 and additionally moves together with FIP200 to peripheral structures after autophagy induction by starvation. Scale bar, 5 μm. (P) PBMM assay, RUBCN KO, starvation induced membrane mixing. The experimental conditions were same as in FIG. 2E,F. (Q,R) HCM quantification, effects of RUBCN KO on starvation-induced (EBSS, 2 h) HyPAS formation. **, p<0.01, (n=3) ANOVA. Scale bar, 10 μm. Masks: white, primary objects (cells), yellow, overlap between FIP200 and ATG16L1. (S,T) HCM, no effect of STX17 KO on colocalization between FLAG-ATG16L1 and LC3 in WT or STX17^{KO} HeLa cells left untreated (DMSO) or treated with monensin (100 μM for 1 h). Masks: white, primary objects (FLAG positive cells), yellow, overlap between LC3 and FLAG-ATG16L1. Scale bar, 10 μm. HCM. **, p<0.01, (n=3) ANOVA. All HCM data, >500 primary objects counted per well; number of wells ≥6/variable/plate; n, number of independent experiments (each on a different plate).

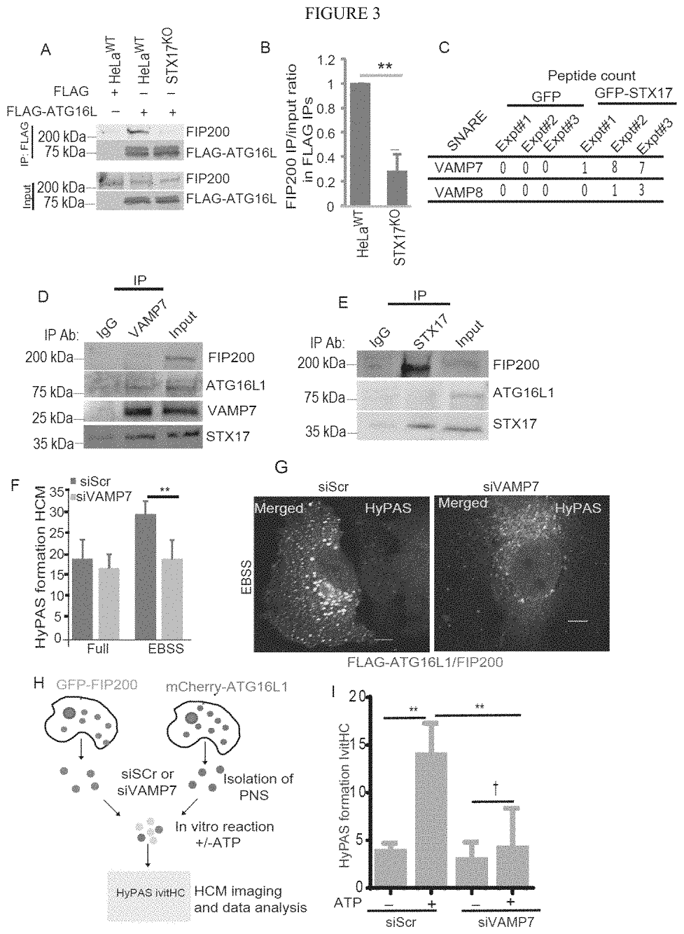

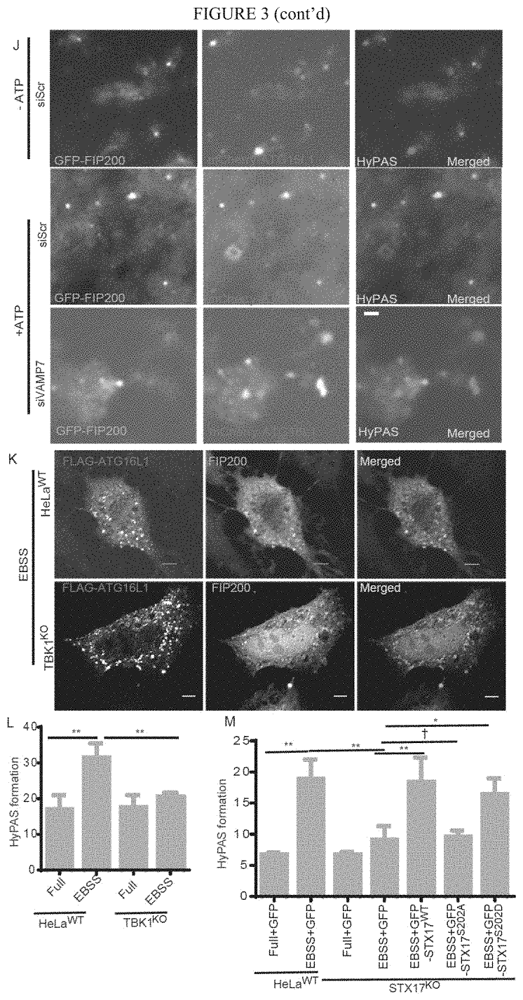

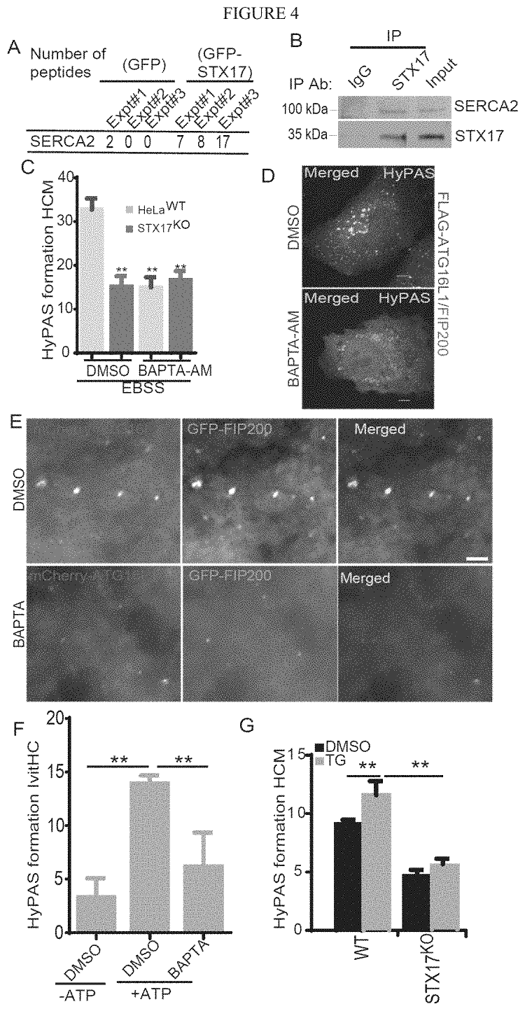

FIGURE S3 shows the dynamics of HyPAS relative to other autophagy markers, related to FIGS. 2 and 3. (A,B) HCM quantification of the effects of STX17 KO on colocalization between mCherry-PIS and FIP200 in HeLa cells incubated with full media or incubated with EBSS for 2 h. Masks: white, primary objects (mcherry-ATG16L1 positive cells), yellow, overlap between FIP200 and mCherry-PIS. **, p<0.01, (n=3) ANOVA. Scale bar, 10 μm. (C) Confocal microscopy, effects of STX17 KO on colocalization between FIP200 and mCherry-PIS. Scale bar, 5 μm. (D) HCM quantification, time course of HyPAS formation and HyPAS profiles positive for GFP-DFCP1 in cells induced for autophagy for indicated times. † p≥0.05, **, p<0.01, (n=3) ANOVA. (E) Confocal microscopy related to ‘D’ showing the effect of starvation (60 min) on GFP-DFCP1 colocalization with HyPAS. White arrows indicate overlap between GFP-DFCP1 and HyPAS. Scale bar, 5 μm. (F) HCM time course of HyPAS formation and HyPAS positivity for GFP-WIPI2b in cells induced for autophagy for indicated times. † p≥0.05, **, p<0.01, (n=3) ANOVA. (G) Confocal microscopy related to ‘F’ showing the effect of starvation (15 min and 60 min) on GFP-WIPI2b positivity of HyPAS. Split channels to the right, zoomed area. Scale bar, 5 μm. (H) HCM, HyPAS formation and HyPAS positivity for LC3 in cells induced for autophagy for indicated times. † p≥0.05, *, p<0.05, **, p<0.01, (n=3) ANOVA. (I) Confocal microscopy, colocalization of HyPAS with LC3 after 60 min of autophagy induction. Scale bar, 5 μm. (J-L) Confocal microscopy and HCM quantification of the effects of STX17 KO on colocalization between LC3 and FLAG-ATG16L1 in cells incubated in full media or induced for autophagy by starvation (EBSS, 2 h). Scale bar, 5 μm (confocal). Masks: white, primary objects (FLAG-ATG16L1 positive cells), yellow, overlap between LC3 and FLAG-ATG16L1. Scale bar, 10 μm (HCM). **, p<0.01, (n=3) ANOVA. (M) Time course HCM analysis of the effects of the simultaneous loss of LC3A,B,C and GABARAP,L1,L2 (Hexa^{KO}) on HyPAS formation in HeLa. † p≥0.05, **, p<0.01, (n=3) ANOVA. (N) Confocal microscopy, no effect of Hexa^{KO} on colocalization between FIP200 and FLAG-ATG16L1 (HyPAS; 1 h starvation). Scale bar, 5 μm. (O) Co-IP analysis, endogenous FIP200 and ATG16L1 in WT or STX17^{KO} HeLa cells. (P) Membrane fractionation on OptiPrep gradients and effects of autophagy induction (EBSS) on redistribution of HyPAS machinery (FIP200, ATG16L1, SIGMAR1 and E-SYT2). (Q) Membrane fractionation on OptiPrep gradients, effect of STX17 KO on redistribution of FIP200 from Golgi in cells upon autophagy induction (EBSS, 2 h). (R) Confocal microscopy, colocalization between endogenous VAMP7 and FLAG-STX17 in HeLa cells. Scale bar, 5 μm. (S) Confocal microscopy, colocalization between VAMP7, FIP200 and FLAG-ATG16L1. Scale bar, 5 μm. (T) HCM images, VAMP7 KD, HyPAS formation. Masks: white, primary objects (FLAG positive cells), yellow, overlap between FIP200 and FLAG-ATG16L1. Scale bar, 10 μm, Western blot, VAMP7KD in cells used for analysis in FIGS. 3G-J and S3T,U. (U) Confocal microscopy, effect of VAMP7KD on colocalization between endogenous FIP200 and FLAG-ATG16L1 in HeLa cells induced for autophagy (EBSS, 2 h). Scale bar, 5 μm. Related to FIG. 3G. All HCM data, >500 primary objects counted per well; number of wells ≥6/variable/plate; n, number of independent experiments (each on a different plate).

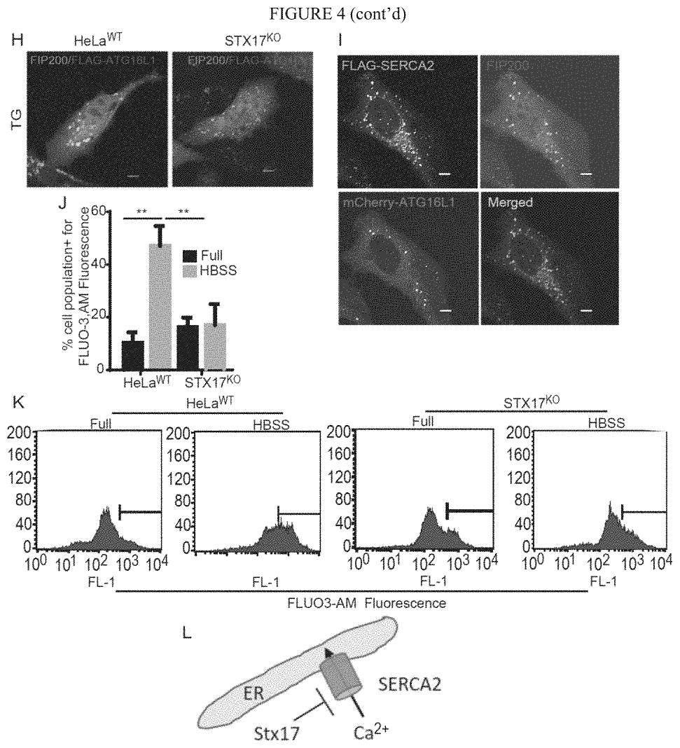

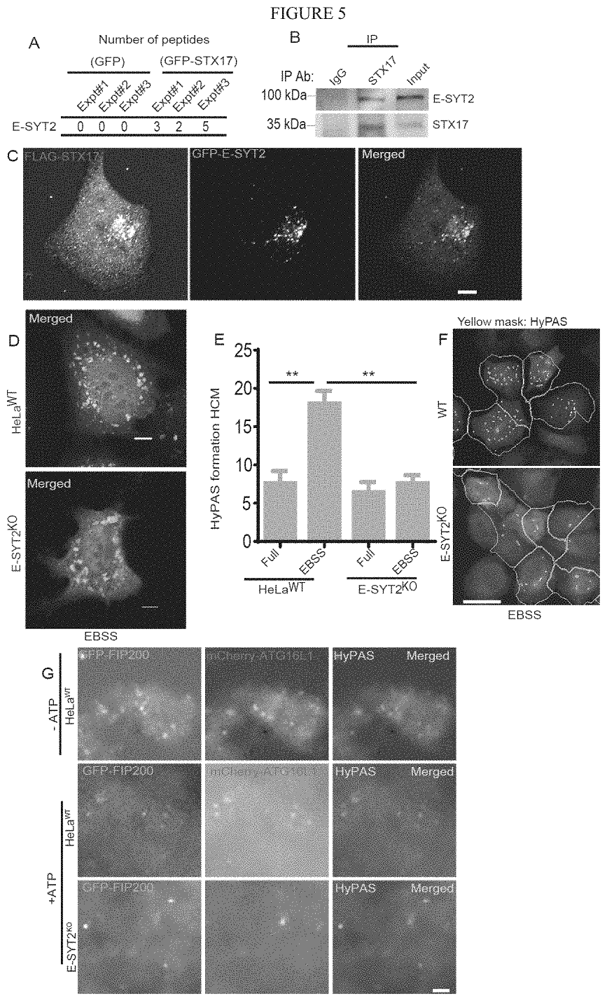

FIGURE S4 shows STX17's SNARE partners and HyPAS formation, related to FIGS. 3 and 4. (A,B) HCM quantification of the effect of VAMP8 KD on HyPAS formation. HCM, minimum number of wells 6; 3 independent experiments. † p≥0.05, **, p<0.01, (n=3) ANOVA. Scale bar, 10 μm. (C) SNAP-47 and SNAP-29 peptides (MS analysis) in GFP or GFP-STX17 complexes. (D) Confocal microscopy, colocalization between GFP-SNAP-47 and FLAG-STX17 in HeLa cells. Scale bar, 5 μm. (E) Co-IP analysis of the interactions between FLAG-STX17 and GFP-SNAP-47. (F) Co-IP analysis of the interactions between GFP-STX17 full length or GFP-STX17 TM (transmembrane region devoid of SNARE domain) with endogenous SNAP-47. (G) HCM, effect of SNAP-29 KD on HyPAS formation. † p≥0.05, **, p<0.01, (n=3) ANOVA. (H-K) Co-IP analyses and quantifications of interactions of wild type STX17, non-phosphorylatable mutant (S202A) of STX17 or phosphomimetic mutant (S202D) of STX17 with VAMP7 or VAMP8. † p≥0.05, **, p<0.01, (n=3) ANOVA. (L) Sample HCM images, transfection of STX17KO cells with WT STX17, non-phosphorylatable mutant of STX17 or phosphomimetic mutant of STX17. Masks: white, primary objects (FLAG and mCherry positive cells), yellow, HyPAS. Scale bar, 10 μm. (M) Co-IP analysis, GFP-STX17 and SERCA2. (N) confocal microscopy, colocalization between endogenous SERCA2 and FLAG-STX17 in HeLa. Scale bar, 5 μm. (O) Confocal microscopy, effect of BAPTA-AM on colocalization between endogenous FIP200 and FLAG-ATG16L1 in HeLa cells induced for autophagy (EBSS, 2 h). Scale bar, 5 μm. Related to FIG. 4D. (P) Sample HCM images, effects of BAPTA-AM treatments in HeLa WT or STX17KO on HyPAS formation. Scale bar, 10 μm. Masks: white, primary objects (FLAG positive cells), yellow, overlap between FIP200 and FLAG-ATG16L1. (Q) Confocal microscopy, effect of thapsigargin treatments on colocalization between endogenous FIP200 and FLAG-ATG16L1 in wild type HeLa or STX17KO cells induced for autophagy (EBSS, 2 h). Scale bar, 5 μm. Related to FIG. 4H. (R) Sample HCM images, effect of thapsigargin treatment in HeLa WT or STX17KO on HyPAS formation. Scale bar, 10 μm. Masks: white, primary objects (FLAG positive cells), yellow, overlap between FIP200 and FLAG-ATG16L1. All HCM data, >500 primary objects counted per well; number of wells ≥6/variable/plate; n, number of independent experiments (each on a different plate).

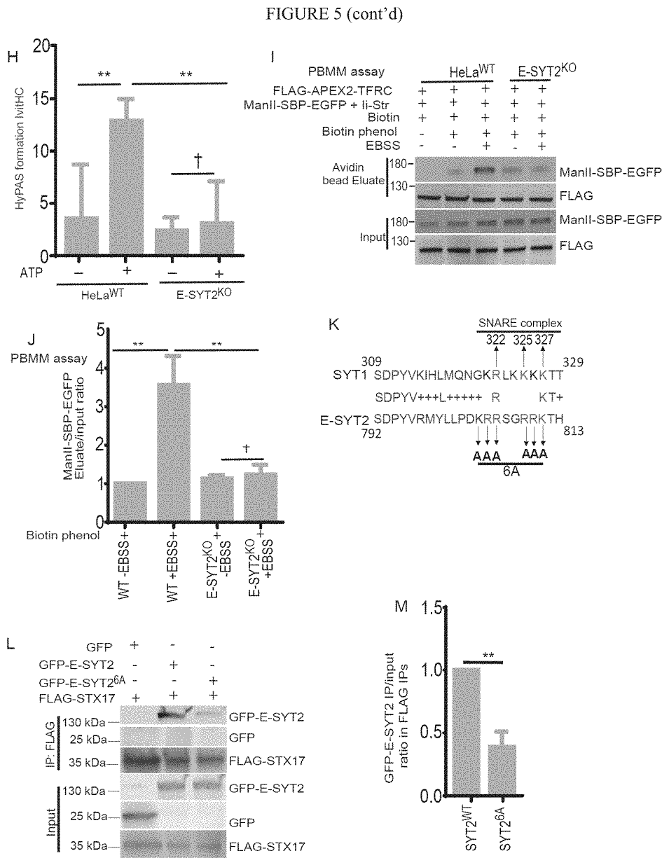

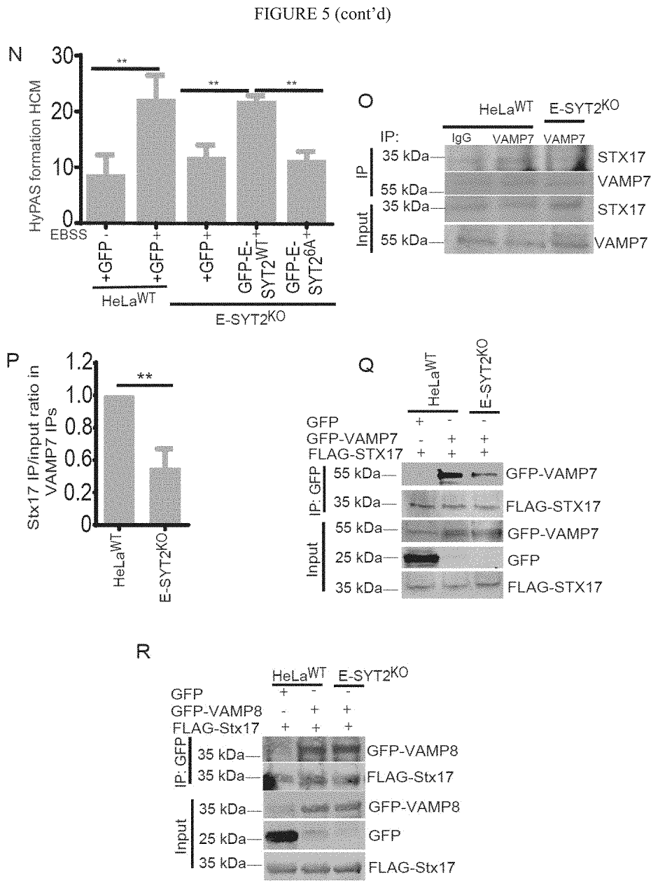

FIGURE S5 shows that E-SYT2 controls HyPAS formation, related to FIG. 5. (A) Co-IP analysis of interactions between GFP-STX17 and E-SYT2 in 293T cells. (B) Confocal microscopy, colocalization between FLAG-E-SYT2, FIP200 and mCherry-ATG16L1 in HeLa cells induced for autophagy (2 h EBSS). Scale bar, 5 μm. (C) Confocal microscopy, effect of E-SYT2KD on colocalization between endogenous FIP200 and FLAG-ATG16L1 in HeLa cells induced for autophagy (EBSS, 2 h). Scale bar, 5 μm. (D,E) HCM quantification, effect of E-SYT2 KD on HyPAS formation in WT or E-SYT2KO HeLa cells. Masks: white, primary objects (FLAG positive cells), yellow puncta, overlap between FIP200 and FLAG-ATG16L1. Scale bar, 10 μm. **, p<0.01, (n=3) ANOVA. (F) Confocal microscopy analysis (split channels related to FIG. 5D) of the effects of E-SYT KO on HyPAS formation (colocalization between FIP200 and FLAG-ATG16L1). Scale bar, 5 μm. (G,H) HCM quantification of the effect of thapsigargin treatment on HyPAS formation in WT or E-SYT2^{KO} HeLa cells. Masks: white, primary objects (FLAG positive cells), yellow, overlap between FIP200 and FLAG-ATG16L1.**, p<0.01, (n=3) ANOVA. Scale bar, 10 μm. (I) Confocal microscopy, complementation of E-SYT2KO cells with GFP-ESYT2WT or GFP-ESYT26A, effects on colocalization between FIP200 and FLAG-ATg16L1 (HyPAS). Scale bar, 5 μm. (J). Co-IP analysis of interactions between FLAG-SIGMAR1 and endogenous VAMP7. (K) HCM quantification (HyPAS formation; overlap between mCherry-ATG16L1 and FIP200), effect of complementation of SIGMAR1KO with empty FLAG, SIGMAR1-FLAG or with SIGMAR1 N80-FLAG transfected into SIGMAR1KO 293A cells. Masks: white, primary objects (FLAG positive cells), yellow, overlap between FIP200 and mCherry-ATG16L1. Scale bar, 10 μm. (L,M) HCM quantification, effects of E-SYT2KO on mitophagy induced by CCCP. Cells were transfected with YFP-Parkin and YFP-Parkin positive cells were gated for quantification of mito-DNA dots (mitophagy). Masks: white, primary objects (YFP-Parkin positive cells), red, mito-DNA dots. **, p<0.01, (n=3) ANOVA. Scale bar, 10 μm. All HCM data, >500 primary objects counted per well; number of wells ≥6/variable/plate; n, number of independent experiments (each on a different plate).

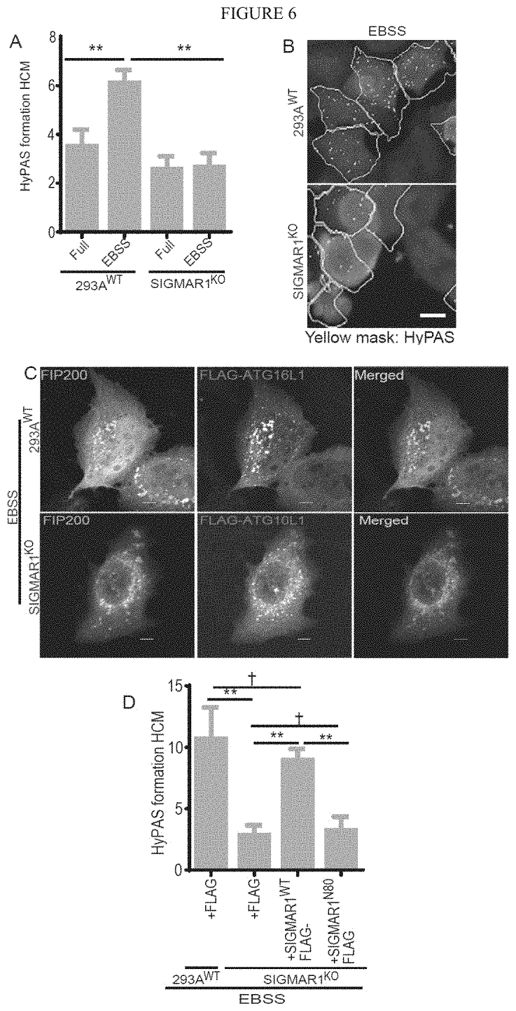

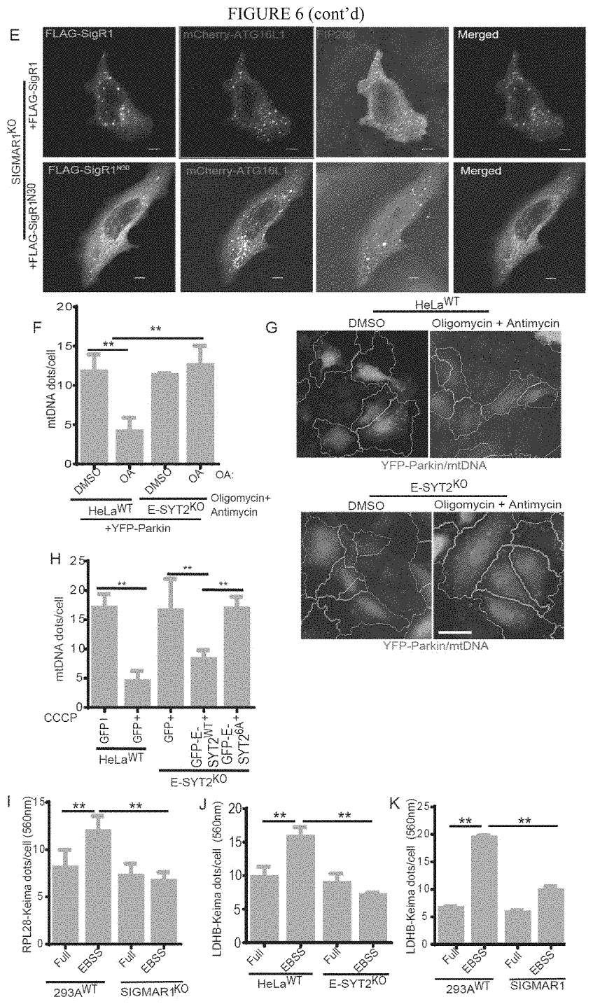

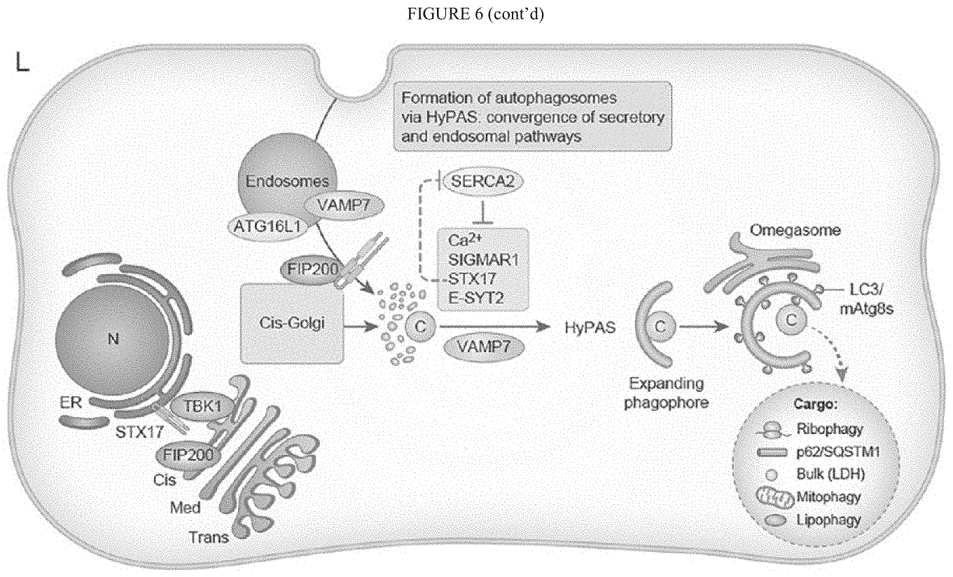

FIGURE S6 shows that E-SYT2 and SIGMAR1 are required for degradation of diverse cargo during autophagy, related to FIG. 6. (A) Sample HCM images, effects of complementation of E-SYT2^{KO} with E-SYT2^{WT} or its 6A mutant E-SYT2^{6A} on mitophagy quantified by mtDNA clearance in mCherry-Parkin⁺ cells. Masks: white, primary objects (GFP and mCherry positive cells), blue, mtDNA dots. Scale bar, 10 μm. (B,C) HCM quantification of effects of SIGMAR1 KO on mitophagy induced by OA treatment in 293A cells. Cells were transfected with YFP-Parkin and YFP-Parkin positive cells were gated for quantification of mito-DNA dots (mitophagy). Scale bar, 10 μm. Masks: white, primary objects (YFP-Parkin positive cells), red, mito-DNA. **, p<0.01, (n=3) ANOVA. (D,E) HCM quantification of the effects of E-SYT2 KO on ribophagy analyzed by ribophagy probe RPL28-Keima. **, p<0.01, (n=3) ANOVA. **, p<0.01, (n=3) ANOVA. Scale bar, 10 μm. Masks: white, primary objects, red, Keima fluorescence at 560 nm; turquoise, Keima fluorescence at 440 nm. (F,G) HCM analysis of the complementation of E-SYT2^{KO} with wild type or 6A mutant of E-SYT2 on ribophagy using RPL-28. Scale bar, 10 μm. † p≥0.05, **, p<0.01, (n=3) ANOVA. (H) HCM images, sample from a large dataset of SIGMAR1 KO effects on ribophagy. Scale bar, 10 μm. Masks: white, primary objects, red, Keima fluorescence at 560 nm; turquoise, Keima fluorescence at 440 nm. (I,J) HCM image samples, SIGMAR1 KO effect on bulk autophagy using LDH-Keima in E-SYT2^{KO} and SIGMAR1^{KO} cells induced for autophagy (8 h, EBSS). Scale bar, 10 μm. Masks: white, primary objects, red, Keima fluorescence at 560 nm; turquoise, Keima fluorescence at 440 nm. (K,L) HCM quantification of the effects of E-SYT2 KO on lipophagy-lipolysis induced by starvation (16 h EBSS) measured by BODIPY in cells preincubated with oleic acid (500 μM for 20 h). Masks: white, primary objects, green, BODIPY fluorescence at 488 nm. Scale bar, 10 μm. † p≥0.05, **, p<0.01, (n=3) ANOVA. (M,N) Western blot analysis and quantification of the effects of E-SYT2 KO on p62 levels in cells induced for autophagy by starvation. *, p<0.05, (n=3) two-way ANOVA. (O,P) Western blot analysis and quantification of the effects of starvation at different time points (2-8 h) on degradation of ManII-EGFP and p62. *, p<0.05, (n=3) ANOVA. (Q,R) Western blot analysis and quantification of effect of E-SYT2 KO on accessibility of p62 to proteinase K (PK; proteinase K protection assay) in cells induced for autophagy in presence of BafA1. 3 independent experiments. *, p<0.05, (n=3) ANOVA. (S,T) HCM quantification of the effects of E-SYT2 KO on CHMP2KD induced CHMP4B dot formation during starvation (EBSS, 2 h). Scale bar, 10 μm. *, p<0.05, (n=3) ANOVA. All HCM data, >500 primary objects counted per well; number of wells ≥6/variable/plate; n, number of independent experiments (each on a different plate).

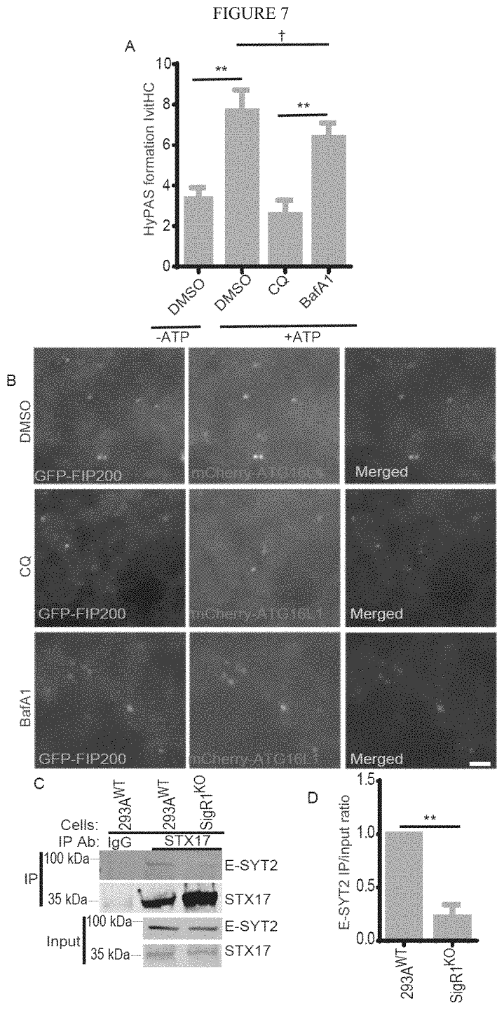

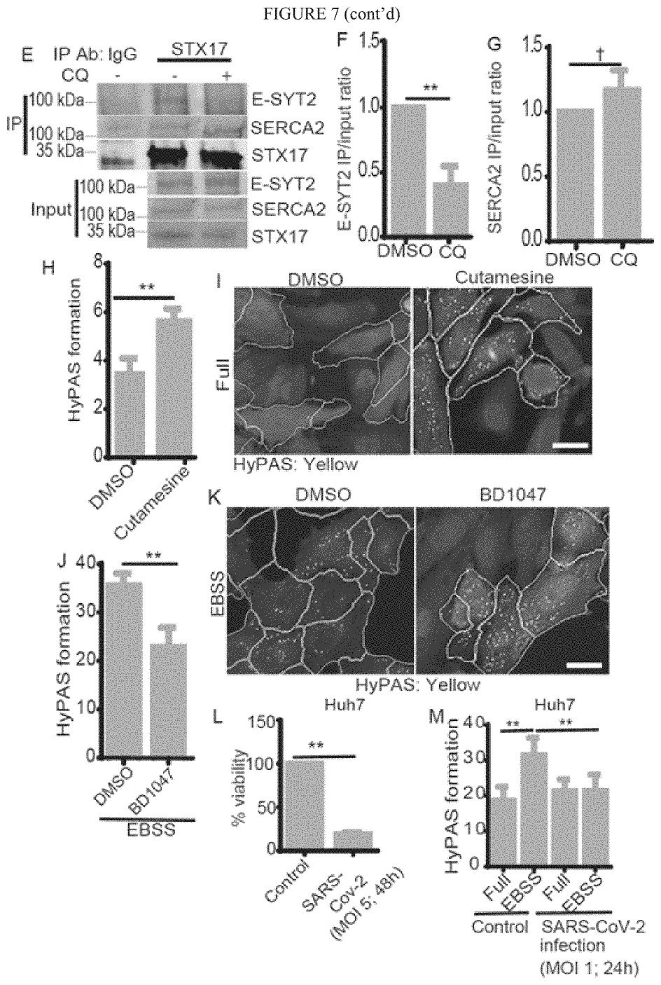

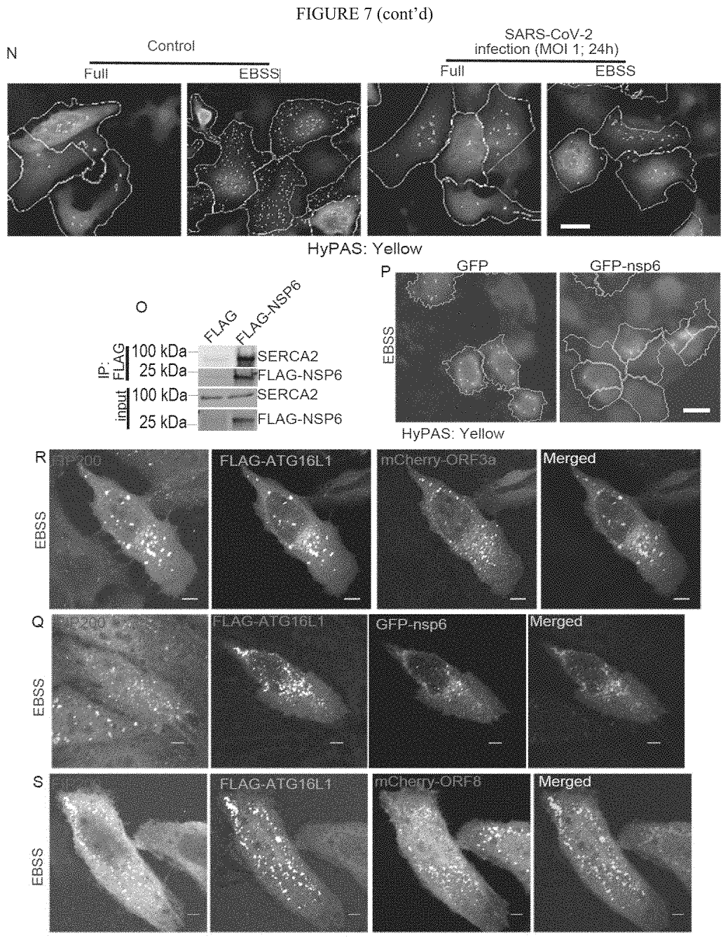

FIGURE S7 shows the effects of pharmacological agents and SARS-CoV-2 on HyPAS, related to FIG. 7. (A,B) Quantification and sample images of the effect of CQ or BafA1 treatments on in vitro fusion assay (ivitHC) between GFP-FIP200 and mCherry-ATG16L1 utilizing high content microscopy. At least 10,000 objects were counted per well and a minimum of 5 wells were used for quantification. † p≥0.05, **, p<0.01, (n=3) ANOVA. Scale bar, 0.5 μm. (C,D) Co-IP analysis, effects of SIGMAR1 KO on interactions of STX17 with E-SYT2 in 293A cells. **, p<0.01, (n=3) t-test. (E-G) Co-IP analysis of effects of CQ treatments on interactions of STX17 with E-SYT2 or SERCA2 in 293T cells. † p≥0.05, **, p<0.01, (n=3) t-test. (H,I) HCM quantification and analysis of the effect of SIGMAR1 agonist cutamesine (100 nM for 2 h) on HyPAS formation. Masks: white, primary objects (FLAG positive cells), yellow, overlap between FIP200 and FLAG-ATG16L1 (HyPAS). **, p<0.01, (n=3) t-test. Scale bar, 10 μm. (J,K) HCM quantification of the effect of SIGMAR1 antagonist BD1407 (10 μM for 1 h) on HyPAS formation in HeLa cell induced for autophagy (EBSS, 2 h). Masks: white, primary objects (FLAG positive cells), yellow, overlap between FIP200 and FLAG-ATG16L1 (HyPAS). Scale bar, 10 μm. **, p<0.01, (n=3) t-test. (L) Cell viability assay showing the effect of SARS-CoV-2 infection (MOI 5; 72 h) on inhibition of viability in Huh7 cells. **, p<0.01, (n=3) t-test. (M,N) HCM quantification of the effects of SARS-CoV-2 infection (MOI: 1, 24 h) on HyPAS formation in Huh7 cells. **p<0.01, (n=3) ANOVA. **, p<0.01, (n=3) ANOVA. Masks: white, primary objects (FLAG positive cells), yellow, overlap between FIP200 and FLAG-ATG16L1 (HyPAS). Scale bar, 10 μm. (O) HCM quantification showing the effect of tetracycline induced expression of APEX2-nsp6^{SARS-COV-2} on the size of starvation-induced LC3 dots. *, p<0.05; **p<0.01, (n=3) ANOVA. (P) Co-IP analysis of interactions between FLAG-nsp6 and SERCA2. (Q) HCM sample images, effect of GFP-nsp6 expression on HyPAS formation. Masks: white, primary objects (GFP-nsp6 positive cells), magenta, overlap between FIP200 and FLAG-ATG16L1 (HyPAS). Scale bar, 10 μm. (R) Confocal microscopy, effect of GFP-nsp6 expression on HyPAS formation. Scale bar, 5 μm. (S,T) Confocal microscopy analysis of the effects of ORF3a expression and ORF8 expression on HyPAS formation. Scale bar, 5 μm. All HCM data in cells, >500 primary objects counted per well; number of wells ≥6/variable/plate; n, number of independent experiments (each on a different plate).

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” includes two or more different compounds. As used herein, the term “include” and its grammatical variants are intended to be non-limiting, such that recitation of items in a list is not to the exclusion of other like items that can be substituted or other items that can be added to the listed items.

The term “compound” or “agent”, as used herein, unless otherwise indicated, refers to any specific chemical compound disclosed herein and includes tautomers, regioisomers, geometric isomers as applicable, and also where applicable, optical isomers (e.g. enantiomers) and isotopomers thereof, as well as pharmaceutically acceptable salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. With respect to cutamesine and BD-1047, the term “compound” also includes isotopomers of each of the these compounds as otherwise disclosed herein. The term also refers, in context to prodrug forms of compounds which have been modified to facilitate the administration and delivery of compounds to a site of activity.

The term “patient” or “subject” is used throughout the specification within context to describe an animal, generally a mammal, including a domesticated mammal including a farm animal (dog, cat, horse, cow, pig, sheep, goat, etc.) and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human.

The terms “effective” or “pharmaceutically effective” are used herein, unless otherwise indicated, to describe an amount of a compound or composition which, in context, is used to produce or affect an intended result, usually the modulation of secretive or degradative autophagy within the context of a particular treatment or alternatively, the effect of a bioactive agent which is coadministered with the secretive or degradative autophagy modulator in the treatment of disease.

The terms “treat”, “treating”, and “treatment”, etc., as used herein, refer to any action providing a benefit to a patient at risk for or afflicted by a secretive or degradative autophagy mediated disease state or condition as otherwise described herein. The benefit may be in curing the disease state or condition, inhibition its progression, or ameliorating, lessening or suppressing one or more symptom of a secretive autophagy mediated disease state or condition. Treatment, as used herein, encompasses both prophylactic and therapeutic treatment. Prophylactic, when used, refers to “reducing the likelihood” of a disease state, condition or symptom associated with same occurring.

The term “co-administration” or “combination therapy” is used to describe a therapy in which at least two active compounds in effective amounts are used to treat a coronavirus infection, including a Covid 19 infection or MERS or other autophagy mediated disease state and/or condition as otherwise described herein, either at the same time or within dosing or administration schedules defined further herein or ascertainable by those of ordinary skill in the art. Although the term co-administration preferably includes the administration of at least two active compounds to the patient at the same time in one or more parts, it is not necessary that the compounds be administered to the patient at the same time, although effective amounts of the individual compounds will be present in the patient at the same time. In addition, in certain embodiments, co-administration will refer to the fact that two or more compounds are administered at significantly different times, but the effects of the two compounds are present at the same time. Thus, the term co-administration includes an administration in which one active agent (especially a modulator of SIGMA-1 Receptor (SIGMAR) as described herein (especially including cutamesine or BD-1047 or an isotopically labeled compound thereof) and optionally, at least one additional autophagy modulator such as ambroxol and/or bromhexine or a mixture thereof as otherwise described herein is administered at approximately the same time (contemporaneously) as the chloroquine, hydroxychloroquine, bafilomycin A1 (BafA1), ivermectin, azithromycin, zinc, molnupiravir, baricitinib, nirmatrelvir and ritonavir (Paxlovid) or a mixture thereof or from about one to several minutes to about 24 hours or more than the other bioactive agent(s).

Compounds used in the methods of treatment of the present invention may be used in pharmaceutical compositions having biological/pharmacological activity for the treatment of, for example, coronavirus, especially SARS, Covid 19 or MERS infections, and a number of other conditions and/or disease states which may appear or occur secondary to the viral infection. These compositions comprise an effective amount of any one or more of the compounds disclosed hereinabove, optionally in combination with a pharmaceutically acceptable additive, carrier or excipient. Compounds used in the methods of treatment of the present invention may also be used as intermediates in the synthesis of compounds exhibiting biological activity as well as standards for determining the biological activity of the present compounds as well as other biologically active compounds.

The term “SIGMA1 receptor modulator” is used to describe a modulator (e.g., an agonist or antagonist of SIGMA1 receptors. Sigma1 receptor antagonists include, for example, haloperidol, BD-1047, BD-1063, 4-IBP, CM-304, NE-100, S1RA (MR-309, API-001), FTC-146, AZ-66, E-52862, or a mixture thereof with BD-1047 or an isotopomer thereof and haloperidol being preferred. These agents, and in particular, BD-1047 and haloperidol, especially including isotopomers of BD-1047 which are otherwise disclosed herein, find particular use in the treatment of SARS-CoV-1, SARS-CoV-2 (Covid19) and MERS-CoV among other infections. SIGMA1 receptor agonists which find use in the treatment of autophagy mediated disease states and/or conditions, including SARS, SARS-CoV-2 (Covid 19) and MERS include fluvoxamine, 4-PPBP (4-phenyl-1-(4-phenylbutylpiperidine)), cutamesine (SA4503) or an isotopomer thereof as disclosed herein, anavex 2-73 (blarcamesine), PRE-084, Ditolylguanidine, dimethyltryptamine, siramesine or a mixture thereof, among others.

In embodiments, the SIGMA1 receptor modulator is often an isotopomer of cutamesine or BD-1047 according to the chemical structures presented herein below. These isotopically labeled agents exhibit great activity and superior pharmacokinetics compared to those same agents which are non-isotopically labeled. Without being limited by way of theory it is believed that these agents exhibit superior pharmacokinetics and greater activity as a consequence of the isotope labeling (generally deuterium for hydrogen in the compounds at key positions of the compounds) which increases the duration of interaction at the SIGMA1 receptor compared to non-isotopically labeled compounds. In addition, these isotopomers exhibit reduced metabolism compared to non-isotopically labeled compound resulting in greater and longer lasting in vivo activity. These agents may be used alone or in combination to effect favorable treatment of SARS, SARS-CoV-2 (COVID 19), MERS and other related autophagy related disease states and/or conditions.

### Cutamesine Isotopomers:

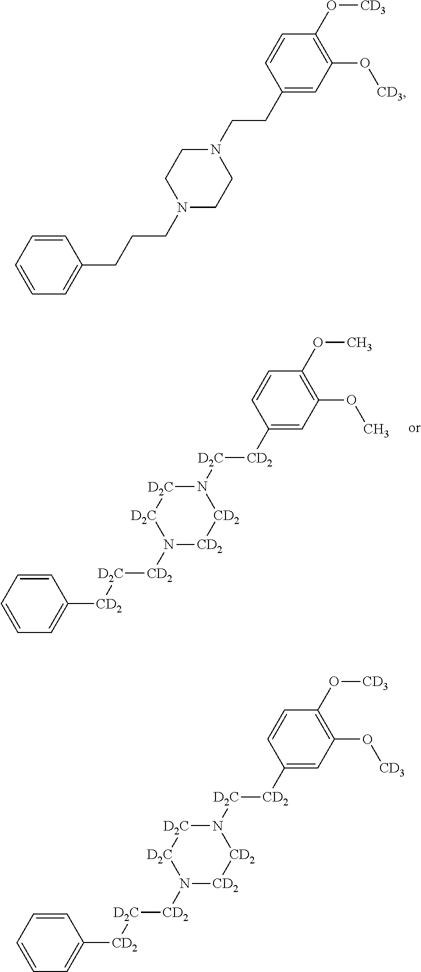

As indicated, in Cutamesine, isotopic labeling is presented in the exocyclic methoxy groups, in the alkylene groups which link the three cyclic groups and/or in the piperazine group.

### BD-1047 Isotopomers:

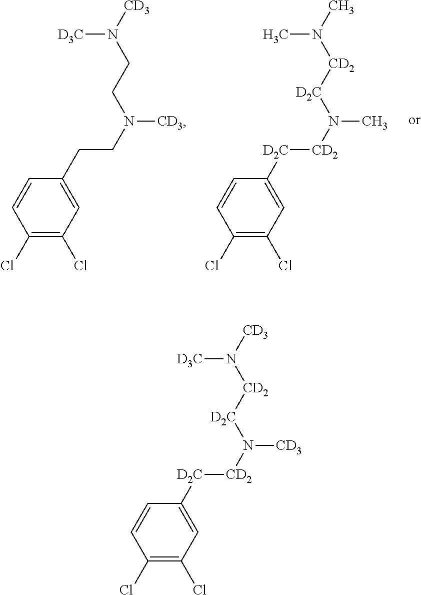

As indicated, in BD-1047, isotopic labeling is presented in the N-methyl groups and/or in the alkylene groups the tertiary amine groups to each other and to the dichlorophenyl group.

In embodiments, isotopomers of Cutamesine or BD-1047 are used alone, in any combination or in combination with non-isotopically labeled Cutamesine and/or BD-1047 and/or any of the other Sigma1 receptor modulators or other additional autophagy modulators which are disclosed herein for the treatment of an autophagy mediated disease state as disclosed herein including SARS, SARS-CoV2 (COVID-19) or MERS. In alternative embodiments, pharmaceutical compositions comprising effective amounts of one or more of these compounds may be used in treating disease states and/or conditions as described herein.

The term “additional autophagy modulator” is used to describe compounds with activity as autophagy agonists and/or antagonists which can be used in combination with a SIGMA1 modulator, especially including Cutamesine, BD-1047 or an isotopomer thereof to provide effective therapy of an autophagy mediated disease state or condition. Additional autophagy modulators include, but are not limited to, autophagy agonists (such as bromhexine, ambroxol, flubendazole, hexachlorophene, propidium iodide, bepridil, clomiphene citrate (Z,E), GBR 12909, propafenone, metixene, dipivefrin, fluvoxamine, dicyclomine, dimethisoquin, ticlopidine, memantine, norcyclobenzaprine, diperodon, nortriptyline or a mixture thereof or their pharmaceutically acceptable salts) to the patient or subject at risk for or suffering from an autophagy mediated disease state and/or condition, especially including a coronavirus infection such as SARS, SARS-CoV2 or MERS. Additional agents which may be used in the present invention to inhibit, prevent and/or treat an autophagy mediated disease state and/or condition, especially including a coronavirus infection such as SARS, SARS-CoV2 or MERS include one or more of benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, Methimazole, Trimeprazine, Ethoxyquin, Clocortolone, Doxycycline, Pirlindole mesylate, Doxazosin, Deptropine, Nocodazole, Scopolamine, Oxybenzone, Halcinonide, Oxybutynin, Miconazole, Clomipramine, Cyproheptadine, Doxepin, Dyclonine, Salbutamol, Flavoxate, Amoxapine, Fenofibrate, Pimethixene and mixtures thereof.

The term “autophagy mediated disease state or condition” is used to describe a disease state or condition that results from disruption in autophagy or cellular self-digestion. Autophagy is a cellular pathway involved in protein and organelle degradation, and has a large number of connections to human disease. Autophagic dysfunction is associated with cancer, neurodegeneration, microbial infection and ageing, among numerous other disease states and/or conditions. Although autophagy plays a principal role as a protective process for the cell, it also plays a role in cell death. Disease states and/or conditions which are mediated through autophagy (which refers to the fact that the disease state or condition may manifest itself as a function of the increase or decrease in autophagy in the patient or subject to be treated and treatment requires administration of an inhibitor or agonist of autophagy in the patient or subject) include, for example, cancer, including metastasis of cancer, lysosomal storage diseases (discussed herein below), neurodegeneration (including, for example, inflammatory disease/disorders, Alzheimer's disease, Parkinson's disease, Huntington's disease; other ataxias), immune response (T cell maturation, B cell and T cell homeostasis, counters damaging inflammation) and chronic inflammatory diseases (may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), affecting lipid metabolism islet function and/or structure, excessive autophagy may lead to pancreatic p-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), and elevated triglycerides) and metabolic syndrome, liver disease (excessive autophagic removal of cellular entities—endoplasmic reticulum), renal disease (apoptosis in plaques, glomerular disease), cardiovascular disease (especially including ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, atherosclerosis and associated conditions such as cardiac and neurological both central and peripheral manifestations including stroke, age-associated dementia and sporadic form of Alzheimer's disease, pre-cancerous states, and psychiatric conditions including depression), stroke and spinal cord injury, arteriosclerosis, infectious diseases (microbial infections, removes microbes, provides a protective inflammatory response to microbial products, limits adaptation of autophagy of host by microbe for enhancement of microbial growth, regulation of innate immunity) including bacterial, fungal, cellular and viral (including secondary disease states or conditions associated with infectious diseases), including AIDS and mycobacterial infections, including tuberculosis, among others, development (including erythrocyte differentiation), embryogenesis/fertility/infertility (embryo implantation and neonate survival after termination of transplacental supply of nutrients, removal of dead cells during programmed cell death) and ageing (increased autophagy leads to the removal of damaged organelles or aggregated macromolecules to increase health and prolong lire, but increased levels of autophagy in children/young adults may lead to muscle and organ wasting resulting in ageing/progeria).

The term “lysosomal storage disorder” refers to a disease state or condition that results from a defect in lysosomal storage. These disease states or conditions include for example, activator deficiency/GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM! Gangliosidosis, including infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs and Wolman disease, among others.

An “inflammatory disorder” “inflammatory disease state” or “inflammatory condition” includes, but is not limited to, lung diseases, hyperglycemic disorders including diabetes and disorders resulting from insulin resistance, such as Type I and Type II diabetes, as well as severe insulin resistance, hyperinsulinemia, and dyslipidemia (e.g. hyperlipidemia (e.g., as expressed by obese subjects), elevated low-density lipoprotein (LDL), depressed high-density lipoprotein (HDL), and elevated triglycerides) and insulin-resistant diabetes, such as Mendenhall's Syndrome, Werner Syndrome, leprechaunism, and lipoatrophic diabetes, renal disorders, such as acute and chronic renal insufficiency, end-stage chronic renal failure, glomerulonephritis, interstitial nephritis, pyelonephritis, glomerulosclerosis, e.g., Kimmelstiel-Wilson in diabetic patients and kidney failure after kidney transplantation, obesity, GH-deficiency, GH resistance, Turner's syndrome, Laron's syndrome, short stature, increased fat mass-to-lean ratios, immunodeficiencies including decreased CD4⁺ T cell counts and decreased immune tolerance or chemotherapy-induced tissue damage, bone marrow transplantation, diseases or insufficiencies of cardiac structure or function such as heart dysfunctions and congestive heart failure, neuronal, neurological, or neuromuscular disorders, e.g., diseases of the central nervous system including Alzheimer's disease, or Parkinson's disease or multiple sclerosis, and diseases of the peripheral nervous system and musculature including peripheral neuropathy, muscular dystrophy, or myotonic dystrophy, and catabolic states, including those associated with wasting caused by any condition, including, e.g., mental health condition (e.g., anorexia nervosa), trauma or wounding or infection such as with a bacterium or human virus such as HIV, wounds, skin disorders, gut structure and function that need restoration, and so forth.

“Inflammatory disorder” also includes a cancer and an “infectious disease” as defined herein, as well as disorders of bone or cartilage growth in children, including short stature, and in children and adults disorders of cartilage and bone in children and adults, including arthritis and osteoporosis. An “inflammation-associated metabolic disorder” includes a combination of two or more of the above disorders (e.g., osteoporosis that is a sequela of a catabolic state). Specific disorders of particular interest targeted for treatment herein are diabetes and obesity, heart dysfunctions, kidney disorders, neurological disorders, bone disorders, whole body growth disorders, and immunological disorders.

In one embodiment, an “inflammatory disorder” includes central obesity, dyslipidemia including particularly hypertriglyceridemia, low HDL cholesterol, small dense LDL particles and postpranial lipemia; glucose intolerance such as impaired fasting glucose; insulin resistance and hypertension, and diabetes. The term “diabetes” is used to describe diabetes mellitus type I or type II. The present invention relates to a method for improving renal function and symptoms, conditions and disease states which occur secondary to impaired renal function in patients or subjects with diabetes as otherwise described herein. It is noted that in diabetes mellitus type I and II, renal function is impaired from collagen deposits, and not from cysts in the other disease states treated by the present invention.

A “neurodegenerative disorder” or “neuroinflammation” includes, but is not limited to inflammatory disorders such as Alzheimer's Dementia (AD), amyotrophic lateral sclerosis, depression, epilepsy, Huntington's Disease, multiple sclerosis, the neurological complications of AIDS, spinal cord injury, glaucoma and Parkinson's disease.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Cancers generally show partial or complete lack of structural organization and functional coordination with the normal tissue and most invade surrounding tissues, metastasize to several sites, and are likely to recur after attempted removal and to cause the death of the patient unless adequately treated. As used herein, the term cancer is used to describe all cancerous disease states applicable to treatment according to the present invention and embraces or encompasses the pathological process associated with all virtually all epithelial cancers, including carcinomas, malignant hematogenous, ascitic and solid tumors. Examples of cancers which may be treated using methods according to the present invention include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, thyroid and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas. See, for example, The Merck Manual of Diagnosis and Therapy, 17.sup.th ed. (Whitehouse Station, N.J.: Merck Research Laboratories, 1999) 973-74, 976, 986, 988, 991).

In addition to the treatment of ectopic cancers as described above, the present invention also may be used preferably to treat eutopic cancers such as choriocarcinoma, testicular choriocarcinoma, non-seminomatous germ cell testicular cancer, placental cancer (trophoblastic tumor) and embryonal cancer, among others.

An “immune disorder” includes, but is not limited to, lupus, multiple sclerosis, rheumatoid arthritis, psoriasis, Type I diabetes, complications from organ transplants, xeno transplantation, diabetes, cancer, asthma, atopic dermatitis, autoimmune thyroid disorders, ulcerative colitis, Crohn's disease, Alzheimer's disease and leukemia.

The terms “severe acute respiratory syndrome coronavirus 1 or 2” (“SARS-CoV-1”) or (“SARS-CoV-2”) and “Middle East respiratory syndrome corona virus” (MERS-CoV)” refer to strains of coronavirus that cause severe acute respiratory syndrome in individuals infected with these viruses. Severe acute respiratory syndrome (SARS) is a viral respiratory illness caused by SARS-associated coronavirus 1 (SARS-CoV-1). SARS was first reported in Asia in February 2003. The illness spread to more than two dozen countries in North America, South America, Europe, and Asia before the SARS global outbreak of 2003 was contained. Middle East Respiratory Syndrome (MERS) is a viral respiratory illness caused by MERS-CoV that is new to humans. It was first reported in Saudi Arabia in 2012 and has since spread to several other countries, including the United States. Most people infected with MERS-CoV developed severe respiratory illness, including fever, cough, and shortness of breath. Many of the infected have died from this illness. COVID 19 is a viral respiratory illness caused by SARS-CoV-2. Most people infected with the virus will experience mild to moderate respiratory illness and recover without requiring special treatment. However, some will become seriously ill and require medical attention. Older people and those with underlying medical conditions like cardiovascular disease, diabetes, chronic respiratory disease, or cancer are more likely to develop serious illness. Anyone can get sick with COVID-19 and become seriously ill or die at any age. In embodiments, the present invention is directed to compositions and methods for the treatment of acute respiratory syndrome caused by SARS-CoV-1, SARS-CoV-2 or MERS-CoV.

The compounds used in the methods of treatment of the present invention may be formulated in a conventional manner using one or more pharmaceutically acceptable carriers. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compounds used in the methods of treatment of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally, or intravenously. Preferred routes of administration include oral administration and pulmonary administration (by inhaler/inhalation spray).

Sterile injectable forms of the compounds used in the methods of treatment of the invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions used in the methods of treatment of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions used in the methods of treatment of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compounds used in the methods of treatment of this invention may also be administered topically, especially when the target of treatment includes areas or organs readily accessible by topical application. Suitable topical formulations are readily prepared for each of these areas or organs.

Topical application also can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions used in the methods of treatment of this invention may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compounds used in the methods of treatment of the instant invention that may be combined with the carrier materials to produce a single dosage form will vary depending upon the host treated, the particular mode of administration. Preferably, the compositions should be formulated so that a therapeutically effective dosage of between about 0.1 and 25 mg/kg, about 1 to about 15 mg/kg of patient/day of the active agent(s) can be administered to a patient receiving these compositions. Preferably, pharmaceutical compositions in dosage form according to the present invention comprise a therapeutically effective amount of at least 25 mg of active agent(s), at least 50 mg of active agent(s), at least 60 mg of active agent(s), at least 75 mg of active agent(s), at least 100 mg of active agent(s), at least 150 mg of active agent(s), at least 200 mg of active agent(s), at least 250 mg of active agent(s), at least 300 mg of active agent(s), about 350 mg of active agent(s), about 400 mg of active agent(s), about 500 mg of active agent(s), about 750 mg of active agent(s), about 1 g (1000 mg) of active agent(s), alone or in combination with a therapeutically effective amount of and/or at least one additional agent selected from the group consisting chloroquine (CQ), hydroxychloroquine HCQ), ivermectin, azithromycin and zinc and/or an additional autophagy modulator.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

Administration of the active compound may range from continuous (intravenous drip) to several oral or inhalation (intratracheal) administrations per day (for example, B.I.D. or Q.I.D.) and may include oral, pulmonary, topical, parenteral, intramuscular, intravenous, sub-cutaneous, transdermal (which may include a penetration enhancement agent), buccal and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Oral dosage forms are particularly preferred, because of ease of administration and prospective favorable patient compliance.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, colouring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques. The use of these dosage forms may significantly the bioavailability of the compounds in the patient.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of compounds according to the present invention.

The present invention also relates to the use of pharmaceutical compositions in an oral dosage form comprising therapeutically effective amounts of isotopically labeled compound according to the present invention, optionally in combination with a pharmaceutically acceptable carrier, additive or excipient. Compositions for oral administration include powders or granules, suspensions or solutions in water or non-aqueous media, sachets, capsules or tablets. Thickeners, diluents, flavorings, dispersing aids, emulsifiers or binders may be desirable.

In embodiments of the invention, especially for treatment of coronavirus, in particular SARS-CoV-2/Covid 19 infections, the compound is administered to the lungs of the subject via pulmonary administration, including intratracheal administration. The pharmaceutical composition of the invention for pulmonary administration is usually used as an inhalant. The composition can be formed into dry powder inhalants, inhalant suspensions, inhalant solutions, encapsulated inhalants and like known forms of inhalants. Such forms of inhalants can be prepared by filling the pharmaceutical composition of the invention into an appropriate inhaler such as a metered-dose inhaler, dry powder inhaler, atomizer bottle, nebulizer etc. before use. Of the above forms of inhalants, powder inhalants may be preferable.

When the pharmaceutical composition used in the methods of treatment of the invention is used in the form of a powder, the mean particle diameter of the powder is not especially limited but, in view of the residence of the particles in the lungs, is preferably that the particles fall within the range of about 0.1 to 20 μm, and particularly about 1 to 5 μm. Although the particle size distribution of the powder pharmaceutical composition of the invention is not particularly limited, it is preferable that particles having a size of about 25 μm or more account for not more than about 5% of the particles, and preferably, 1% or less to maximize delivery into the lungs of the subject.

The pharmaceutical composition in the form of a powder can be produced by, for example, using the drying-micronization method, the spray drying method and standard pharmaceutical methodology well known in the art.

By way of example without limitation, according to the drying-pulverization method, the pharmaceutical composition in the form of a powder can be prepared by drying an aqueous solution (or aqueous dispersion) containing the compound or mixtures with other active agents thereof and excipients which provide for immediate release in pulmonary tissue and microparticulating the dried product. Stated more specifically, after dissolving (or dispersing) a pharmaceutically acceptable carrier, additive or excipient in an aqueous medium, compounds according to the present invention in effective amounts are added and dissolved (or dispersed) by stirring using a homogenizer, etc. to give an aqueous solution (or aqueous dispersion). The aqueous medium may be water alone or a mixture of water and a lower alcohol. Examples of usable lower alcohols include methanol, ethanol, 1-propanol, 2-propanol and like water-miscible alcohols. Ethanol is particularly preferable. After the obtained aqueous solution (or aqueous dispersion) is dried by blower, lyophilization, etc., the resulting product is pulverized or microparticulated into fine particles using jet mills, ball mills or like devices to give a powder having the above mean particle diameter. If necessary, additives as mentioned above may be added in any of the above steps.

According to the spray-drying method, the pharmaceutical composition in the form of a powder of the invention can be prepared, for example, by spray-drying an aqueous solution (or aqueous dispersion) containing isoniazid, urea or mixtures thereof and excipients, additives or carriers for microparticulation. The aqueous solution (or aqueous dispersion) can be prepared following the procedure of the above drying-micronization method. The spray-drying process can be performed using a known method, thereby giving a powdery pharmaceutical composition in the form of globular particles with the above-mentioned mean particle diameter.

The inhalant suspensions, inhalant solutions, encapsulated inhalants, etc. can also be prepared using the pharmaceutical composition in the form of a powder produced by the drying-micronization method, the spray-drying method and the like, or by using a carrier, additive or excipient and isoniazid, urea or mixtures thereof that can be administered via the lungs, according to known preparation methods.

Furthermore, the inhalant comprising the pharmaceutical composition of the invention is preferably used as an aerosol. The aerosol can be prepared, for example, by filling the pharmaceutical composition of the invention and a propellant into an aerosol container. If necessary, dispersants, solvents and the like may be added. The aerosols may be prepared as 2-phase systems, 3-phase systems and diaphragm systems (double containers). The aerosol can be used in any form of a powder, suspension, solution or the like.

Examples of usable propellants include liquefied gas propellants, compressed gases and the like. Usable liquefied gas propellants include, for example, fluorinated hydrocarbons (e.g., CFC substitutes such as HCFC-22, HCFC-123, HFC-134a, HFC-227 and the like), liquefied petroleum, dimethyl ether and the like. Usable compressed gases include, for example, soluble gases (e.g., carbon dioxide, nitric oxide), insoluble gases (e.g., nitrogen) and the like.

The dispersant and solvent may be suitably selected from the additives mentioned above. The aerosol can be prepared, for example, by a known 2-step method comprising the step of preparing the composition of the invention and the step of filling and sealing the composition and propellant into the aerosol container.

As a preferred embodiment of the aerosol according to the invention, the following aerosol can be mentioned: Examples of the compounds to be used include isotopically labeled compound alone or in mixtures with other compounds according to the present invention or with other agents including additional autophagy modulators. As propellants, fluorinated hydrocarbons such as HFC-134a, HFC-227 and like CFC substitutes are preferable. Examples of usable solvents include water, ethanol, 2-propanol and the like. Water and ethanol are particularly preferable. In particular, a weight ratio of water to ethanol in the range of about 0:1 to 10:1 may be used.

The aerosol of the invention contains excipient in an amount ranging from about 0.01 to about 10⁴ wt. % (preferably about 0.1 to 10³ wt. %), propellant in an amount of about 10² to 10⁷ wt. % (preferably about 10³ to 10⁶ wt. %), solvent in an amount of about 0 to 10⁶ w % (preferably about 10 to 10⁵ wt. %), and dispersant in an amount of 0 to 10³ wt. % (preferably about 0.01 to 10² wt. %), relative to the weight of compound according to the present invention which is included in the final composition.

The pharmaceutical compositions of the invention are safe and effective for use in the therapeutic methods according to the present invention. Although the dosage of the compounds used in the methods of treatment of the invention may vary depending on the type of active substance administered as well as the nature (size, weight, etc.) of the subject to be diagnosed, the composition is administered in an amount effective for treating a coronavirus infection, and in particular SARS, Covid 19 or MERS. For example, the composition is preferably administered such that the active ingredient can be given to a human adult in a dose of at least about 25 mg, at least about 50 mg, at least about 60 mg, at least about 75 mg, at least about 100 mg, at least about 150 mg, at least about 200 mg, at least about 250 mg, at least about 300 mg, at least about 350 mg, at least about 400 mg, at least about 500 mg, at least about 750 mg, at least about 1000 mg, and given in a single dose, including sustained or controlled release dosages once daily.

The form of the pharmaceutical composition of the invention such as a powder, solution, suspension etc. may be suitably selected according to the type of substance to be administered.

As an administration route, direct inhalation via the mouth using an inhaler is usually administered into the airways and in particular, directly to pulmonary tissue, the active substance contained therein produces immediate effects. Furthermore, the composition is formulated as an immediate release product so that cleavage and analysis can begin soon after administration.

### Examples

Experiments related to the present invention are described in detail herein below.

### Chemical Synthesis of Isotopomeric Cutamesine and BD-1047

### Cutamesine

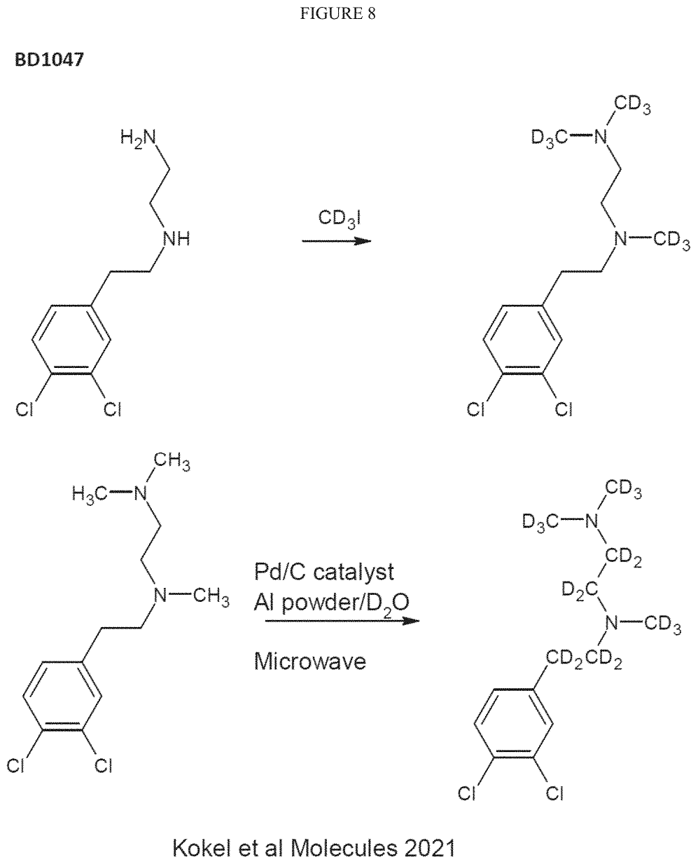

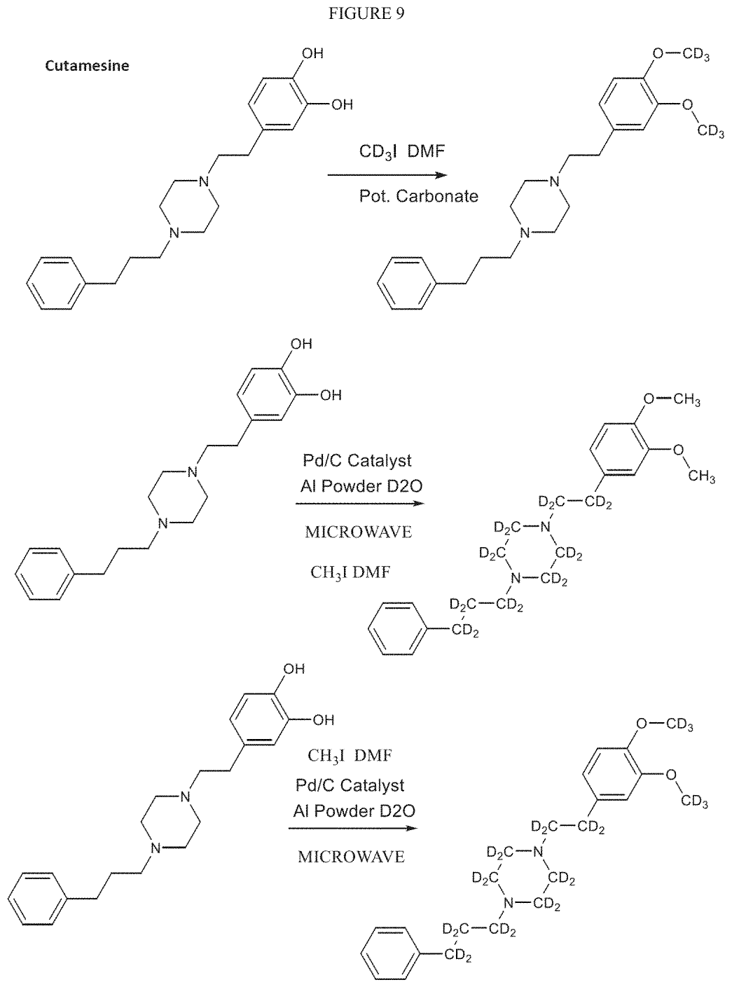

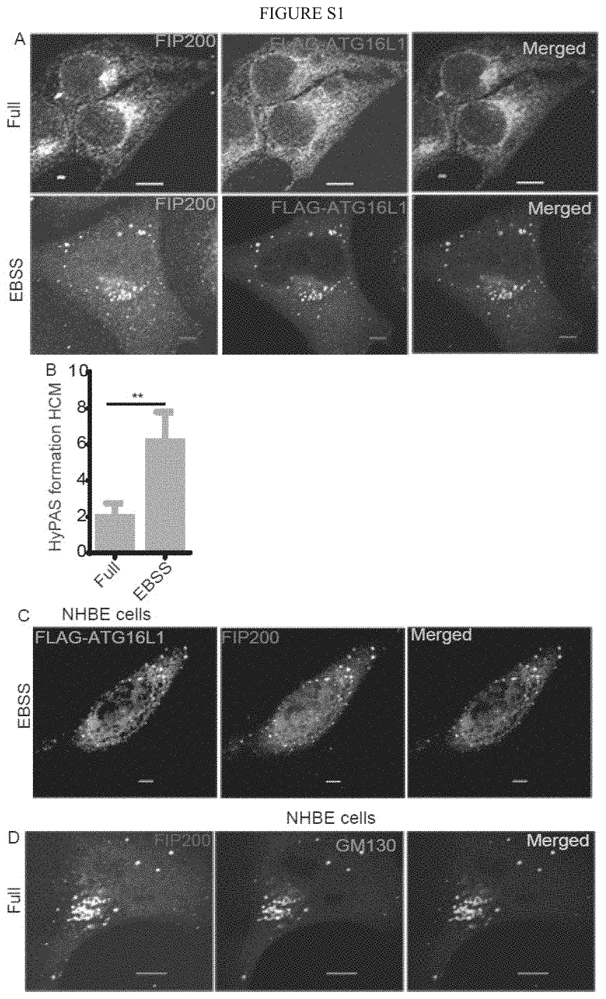

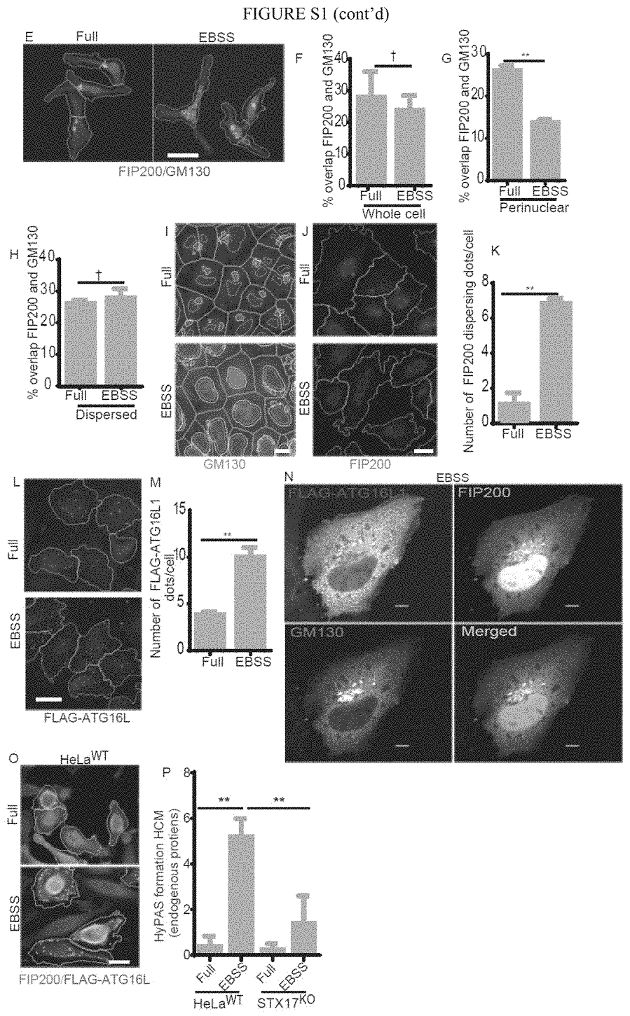

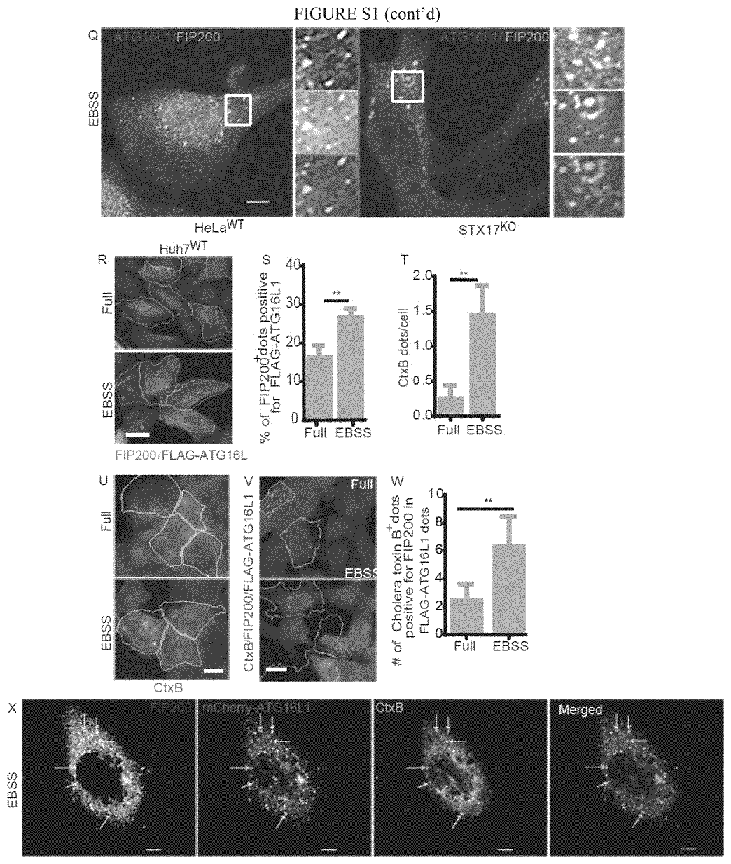

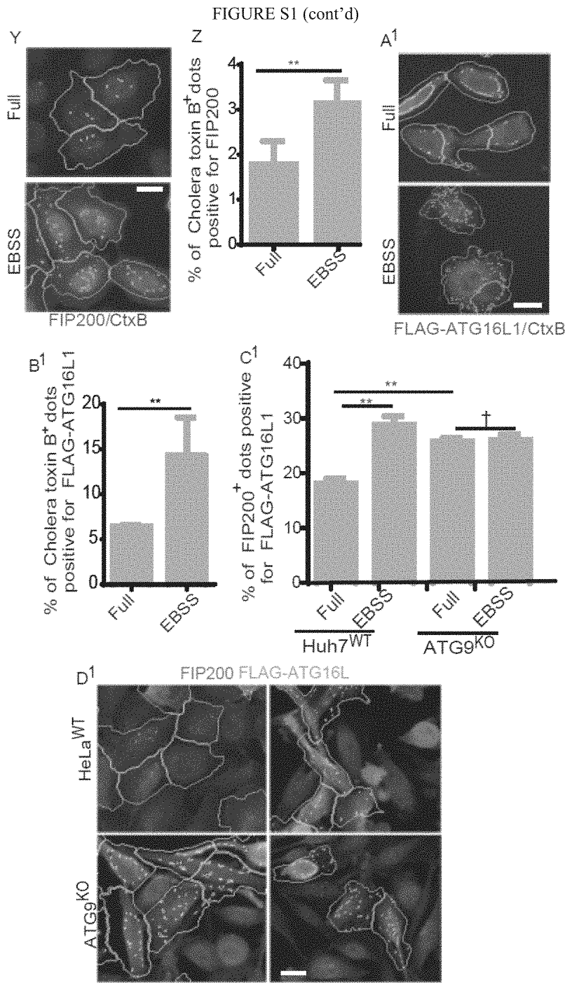

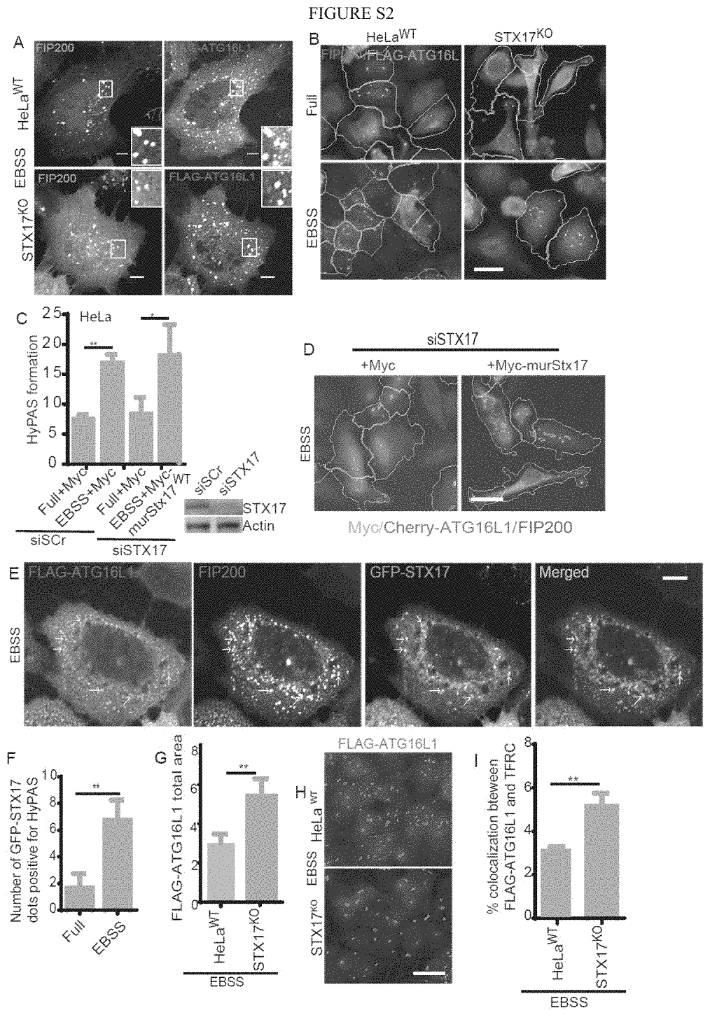

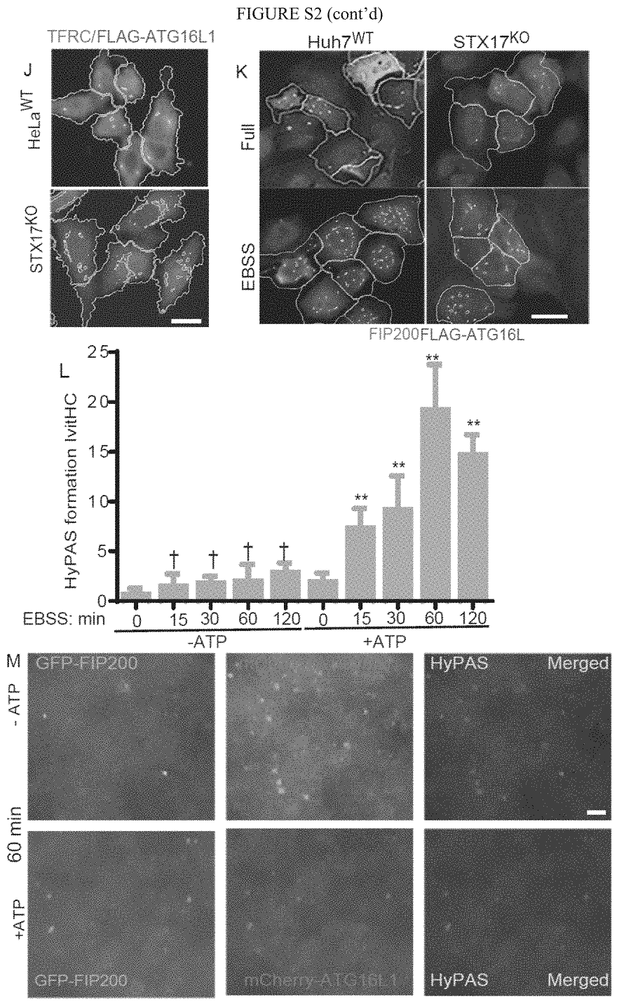

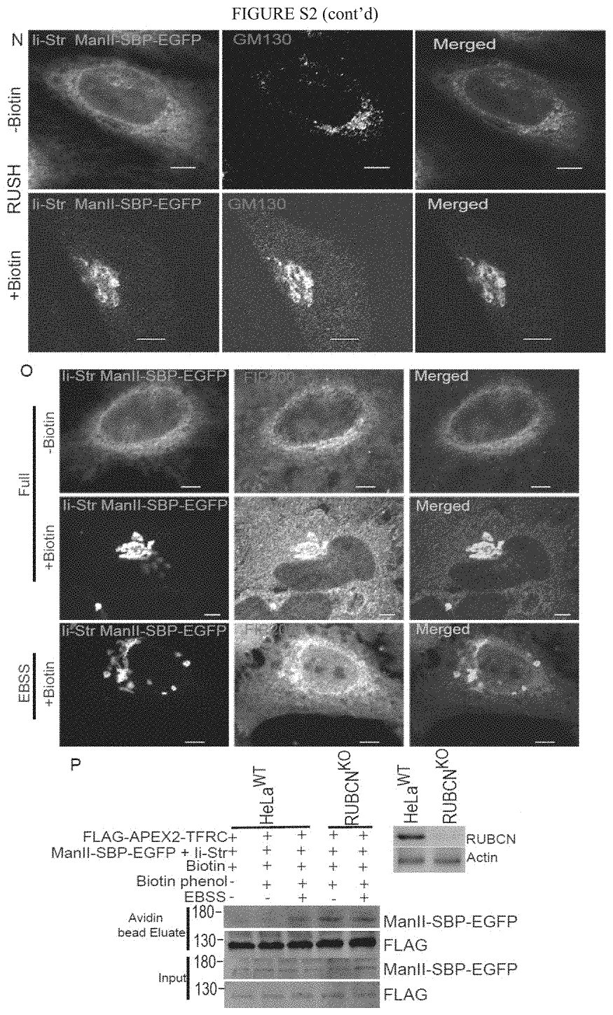

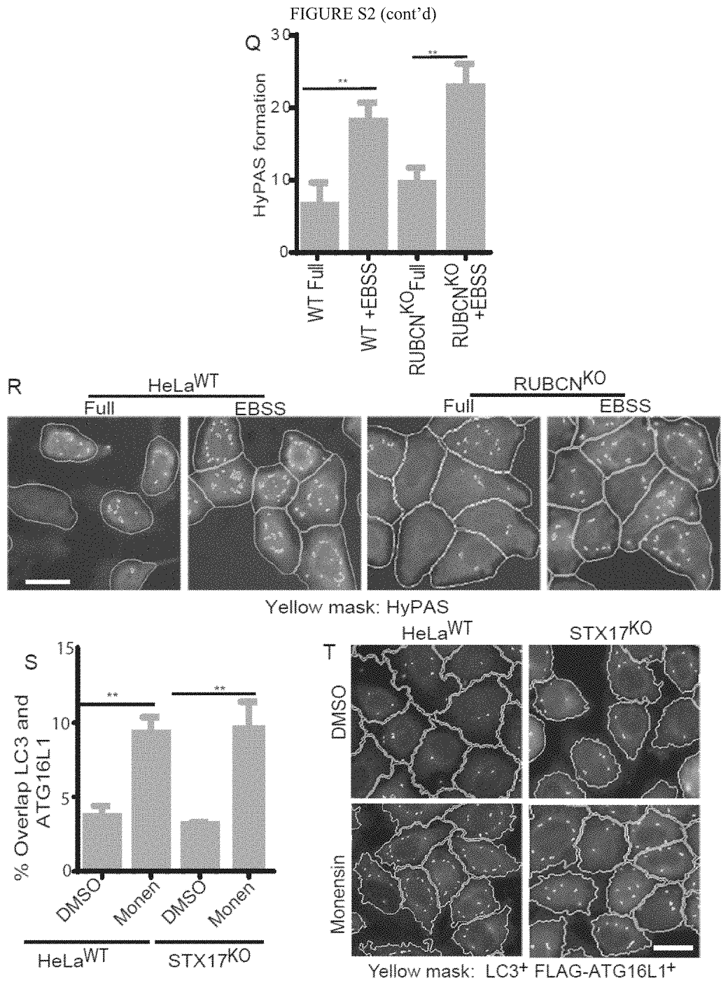

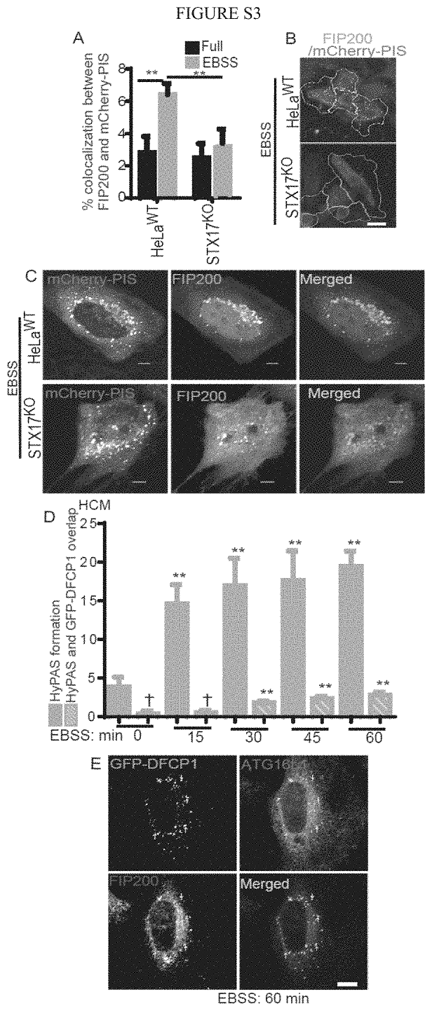

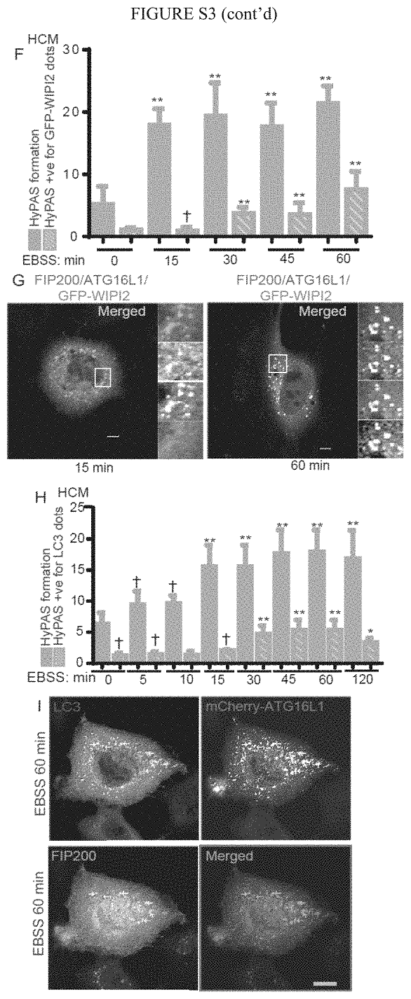

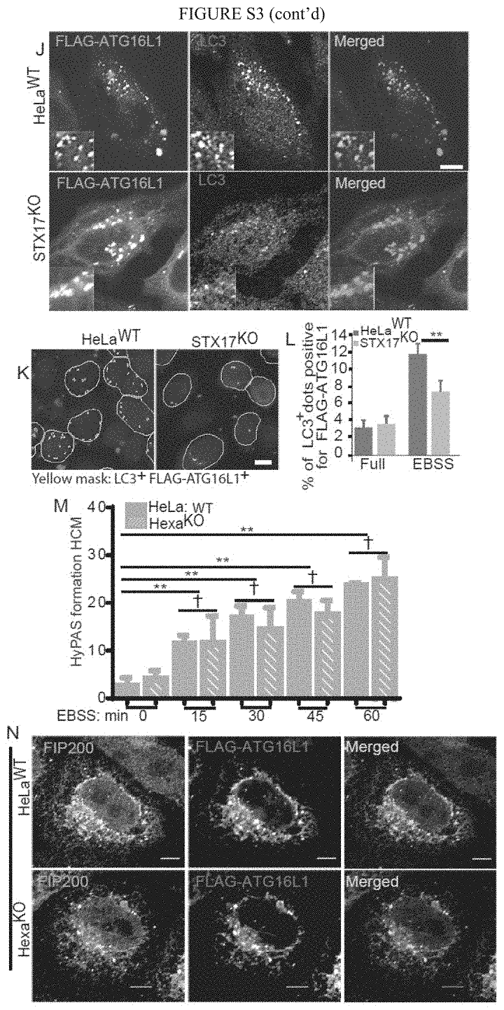

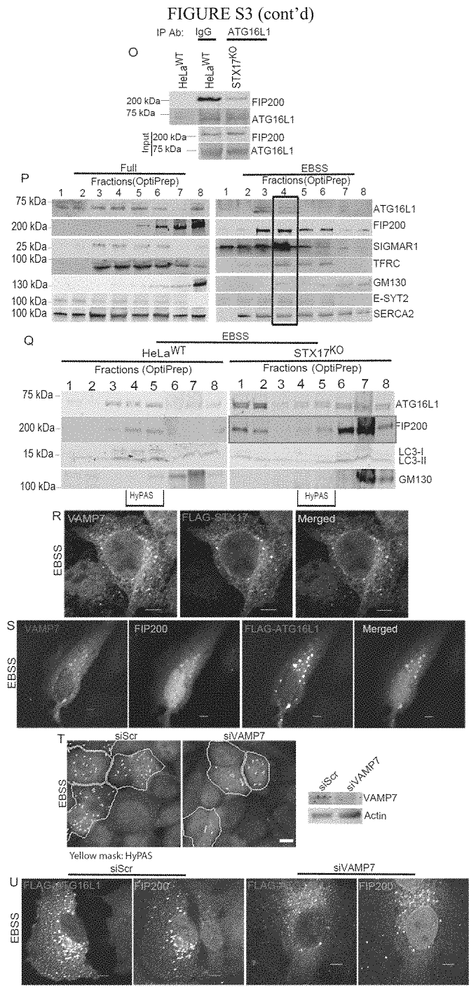

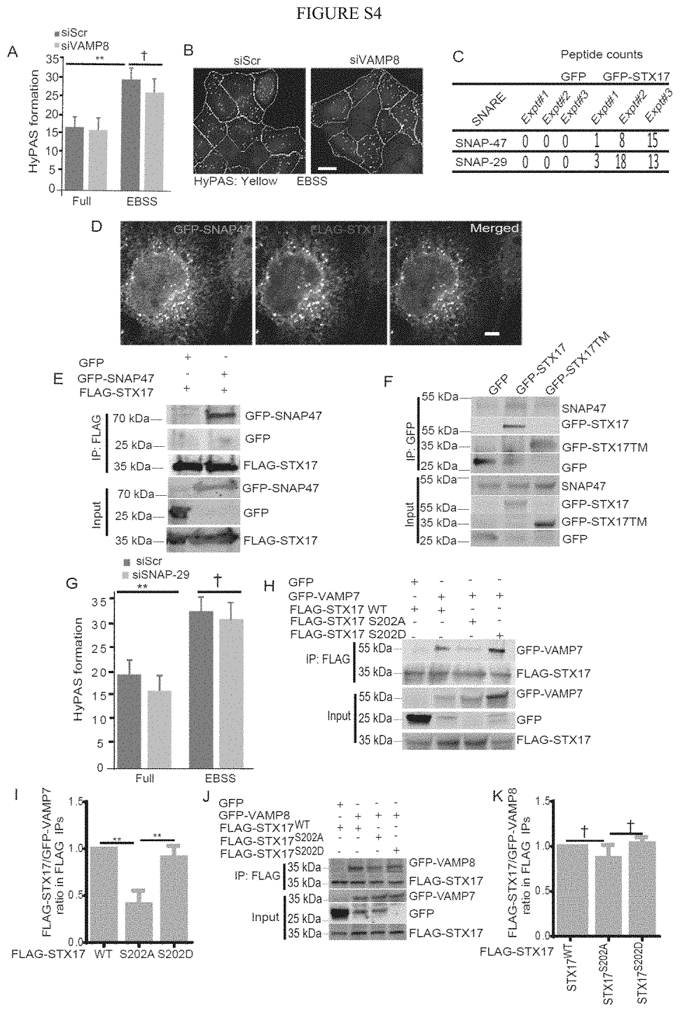

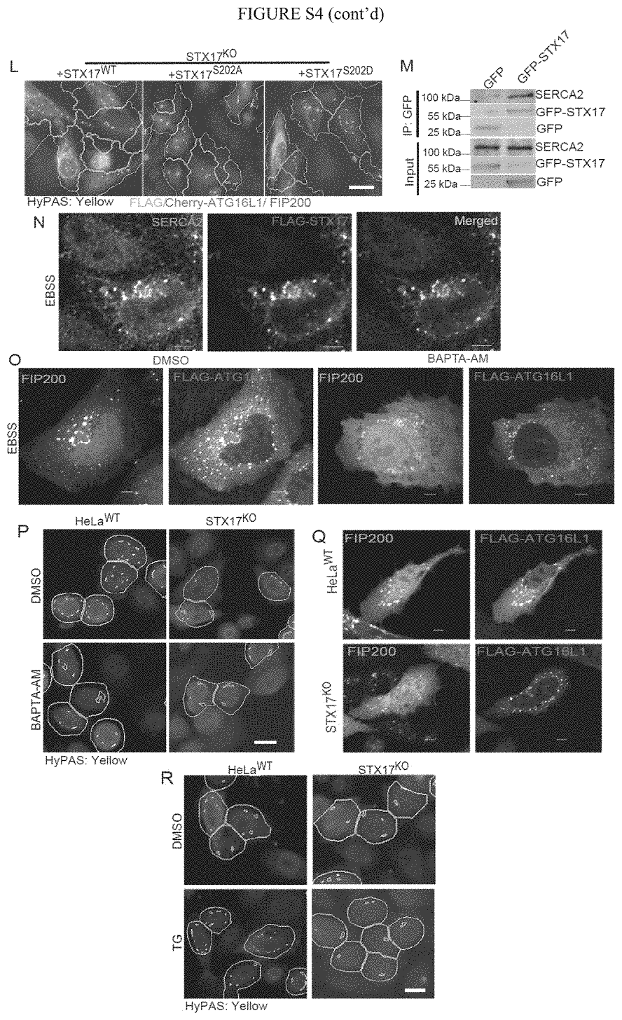

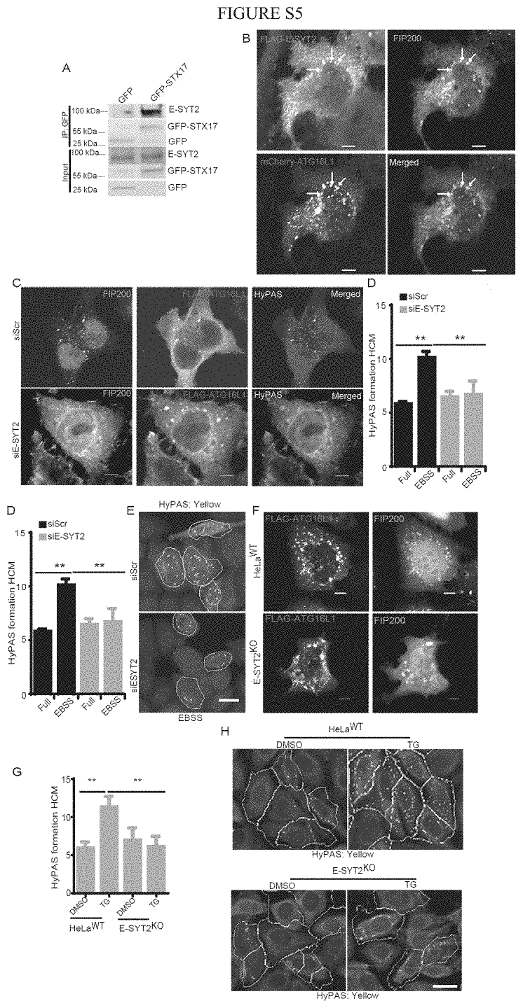

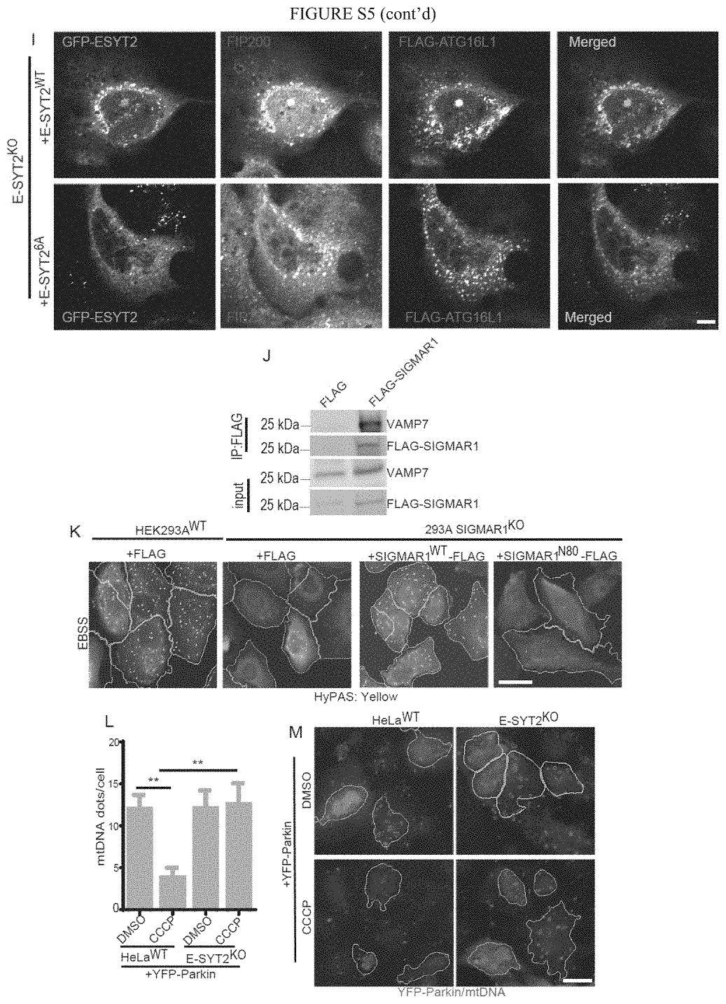

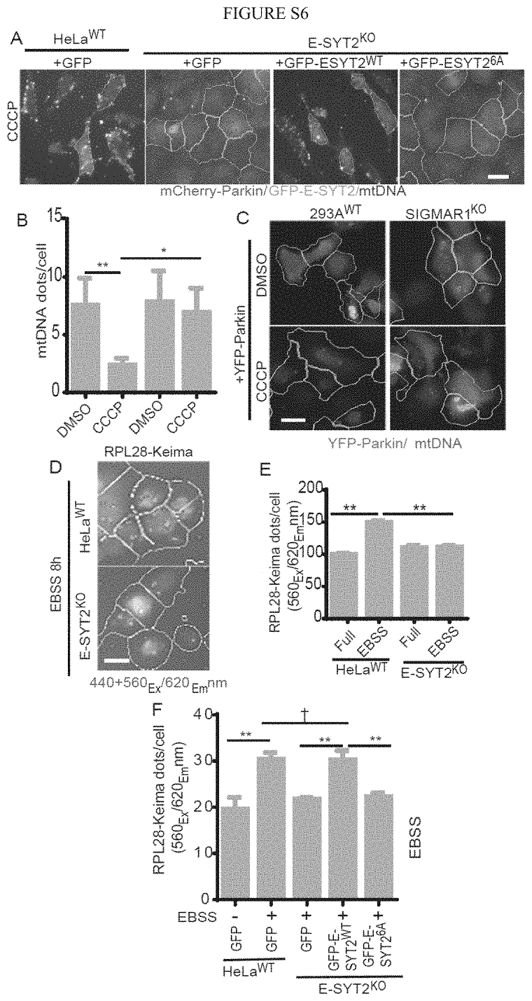

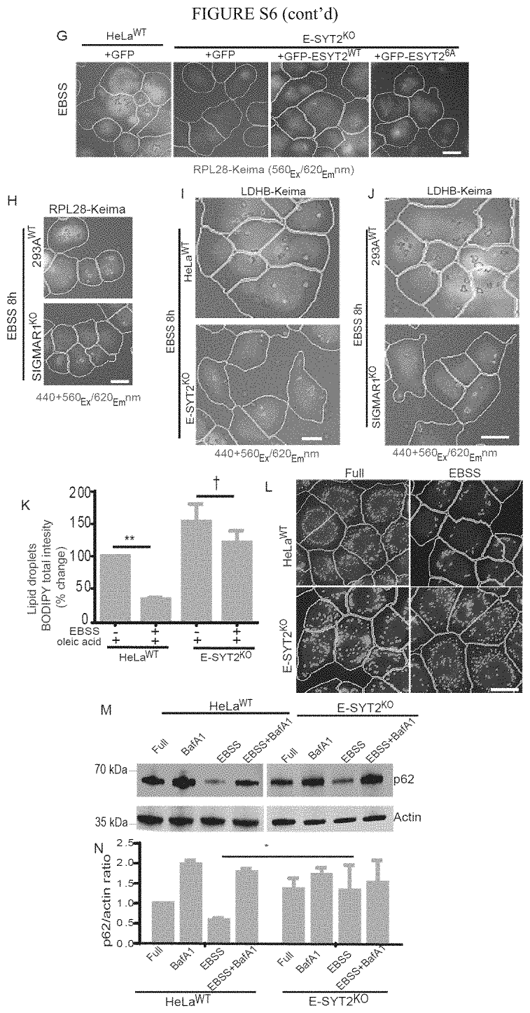

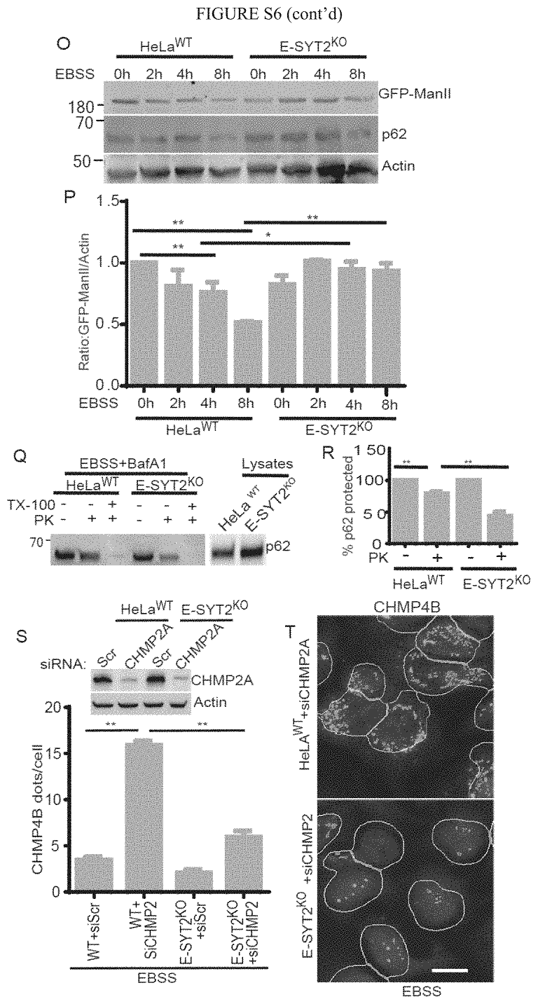

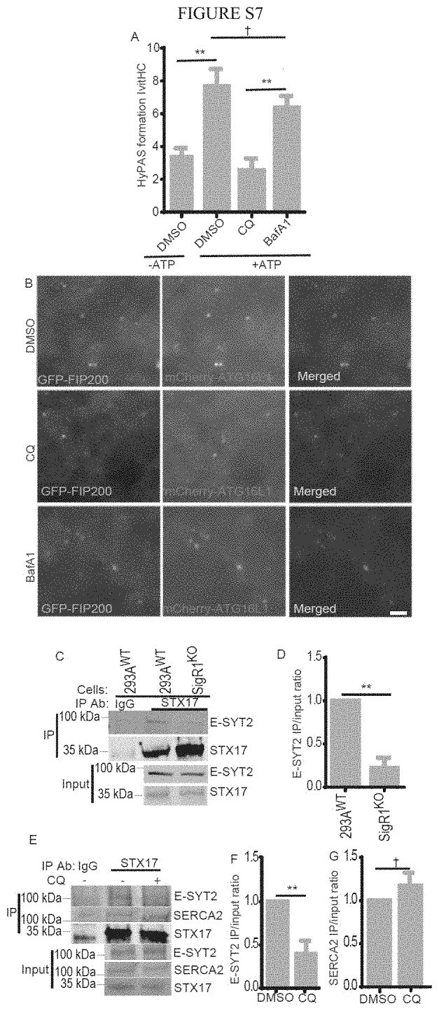

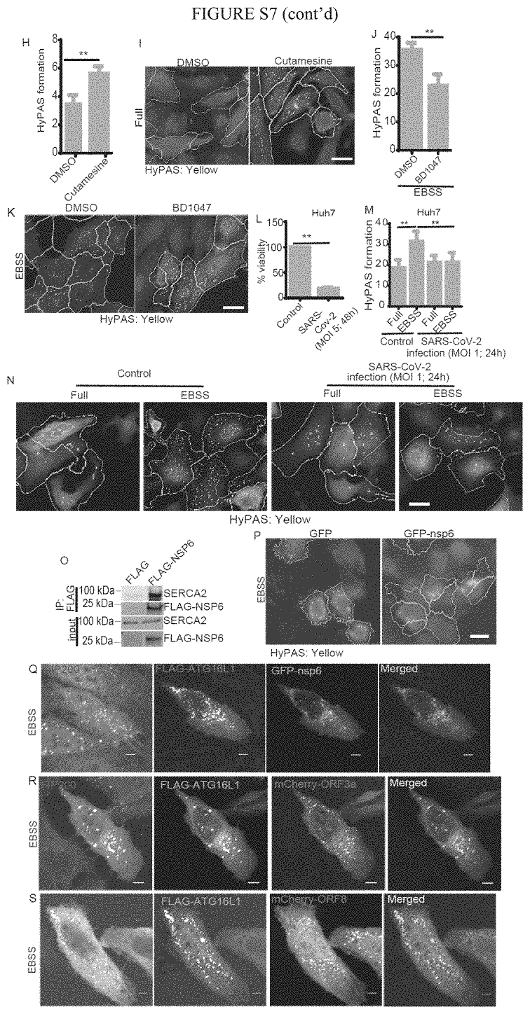

FIGS. 8 and 9 show the facile chemical synthesis of isotopomeric forms of BD-1047 (FIG. 8) and cutamesine (FIG. 9), respectively. The synthesis follows the methods which are set forth in Kokel, et al., Molecules 2021. Pursuant to this approach, deuterated alkyl groups may be introduced onto nucleophilic groups by reacting a deuterated alkyl iodide (e.g. CD₃I) with the nucleophilic group (e.g. OH, NH₂) to provide a deuterated alkyl group. In addition, alkyl and alkylene groups may be deuterated using palladium/carbon catalyst, aluminum powder and deuterated water (D₂O) in the presence of microwave radiation. These steps may be used as depicted in FIGS. 8 and 9, or in conjunction with other standard chemical synthetic steps to afford the various isotopomeric forms of BD-1047 and cutamesine.

### Material and Methods

### Star Methods

### Data and Code Availability

The Raw MS DIA/DDA data used in this study have been deposited at the MassIVE proteomics repository https://massive.ucsd.edu MSV000083251 and MSV000087840.
This paper does not report original code.
Any additional information required to reanalyze the data reported in this paper is available from the lead contact upon request.

### Experimental Model and Subject Details

### Cell Culture

HEK 293T, Huh7 and HeLa cells were obtained from ATCC and maintained in ATCC recommended media. 293T APEX2-nsp6 cells were grown in DMEM supplemented with 10% fetal bovine serum and antibiotic. STX17^{KO} HeLa, FIP200^{KO} HeLa, RUBCN^{KO} HeLa, Hexa^{KO} HeLa, TBK1^{KO} HeLa and STX17^{KO} Huh7, ATG9^{KO} Huh7 cells were cultured in DMEM supplemented with 10% fetal bovine serum and antibiotic as described previously (Gu et al., 2019; Kumar et al., 2019). E-SYT2^{KO} and parental wild type HeLa cells were cultured as described previously (Saheki et al., 2016). HEK293A SIGMAR1^{KO} and parental wild type HEK293A cells were grown as described previously (Yang et al., 2019). Primary NHBE (normal human bronchial epithelial) cells were obtained from Lonza and cultured in media from Lonza (BEGM™ bronchial epithelial cell growth medium BulletKit™; Catalog #: CC-3170).

### Generation of CRISPR Mutant Cells

STX17 CRISPR in HeLa and Huh7 cells and ATG9 CRISPR in Huh7 were generated as described earlier (Gu et al., 2019; Kumar et al., 2019; Kumar et al., 2018). Briefly, the lentiviral vector carrying both Cas9 enzyme and a gRNA targeting STX17 (SEQ ID NO. 1: GATAGTAATCCCAACAGACC), and ATG9 (SEQ ID NO. 1: GACCCCCAGGAGTGTGACGG) were transfected into HEK293T cells together with the packaging plasmids psPAX2 and pCMV-VSV-G at the ratio of 5:3:2. Two days after transfection, the supernatant containing lentiviruses was collected and used to infect the cells. 36 hours after infection, the cells were treated with puromycin (1 mg/ml) for one week to select STX17-knockout cells. The knockouts were confirmed by western blotting. SIGMAR1^{KO} in HEK293A are described previously (Yang et al., 2019), E-SYT2KO cells are described previously (Saheki et al., 2016).

### Generation of 293T Flp-In-nsp6^{TetON} Cells

293T Flp-In host cells were transfected with FLAG-APEX2-nsp6 plasmid and the pOG44 expression plasmid at ration of 9:1. After overnight transfection, cells were washed with PBS and fresh medium was added. 48 h after transfection, cells were trypsinized and seeded at 25% confluency, followed by incubation at 37° C. for 8 h. Cells were then supplied with medium containing 100 μg/mL hygromycin and hygromycin-resistant clones were selected. The clones were tested by western blotting. The tested clones incubated in the medium containing 1 μg/mL tetracycline overnight were determined by western blot with FLAG.

### Method Details

### Antibodies and Reagents

The following antibodies and dilutions were used: STX17 (Sigma, HPA001204; 1:1000 (WB)); Flag (mouse monoclonal Sigma; F1804, used at 0.5 μg/ml and 1:1,000 for (WB); 1:250 (IF)); GFP (rabbit Abcam; ab290; 0.5 μg/ml IP and 1:4,000 (WB)); LC3 (rabbit; MBL International PM036, 1:500 (IF); FIP200 (rabbit; Proteintech; 17250-1-AP, 1:200 (IF)); FIP200 (rabbit Cell Signaling Technology; 12436, 1:1000 (WB)); SERCA2 (Mouse ThermoFisher; MA3-919, 1:150 (IF), 1:500 (WB)); ATG16L1 (mouse; MBL International M152-3, 1:200 (IF); ATG16L1 (rabbit; MBL International PM040, 1:1000 (WB); GM 130 (mouse BD Biosciences, RUO-610822, 1:500 (IF); 1:1000 (WB)); mouse anti-DNA antibody (IF:1:200) was purchased from Progen (#61014); VAMP7 (rabbit Cell Signaling Technology; 14811, 1:1000 (WB)); VAMP8 (1:1,000 (WB); rabbit monoclonal; ab76021; Abcam); E-SYT2 (rabbit ThermoFisher PA5-51689, 1:200 (IF), 1:500 (WB)); GRP78/Bip (1:200 (IF); goat polyclonal; sc10086; Santa Cruz Biotechnology, Inc.); Dynabeads Protein G (Thermo Fisher Scientific 10003D 50 μl/IP); Bafilomycin A1 (Baf A1, InvivoGen, 13D02-MM); OptiPrep Density Gradient Medium (Sigma, D1556); Lipofectamine 2000, Thermo Scientific, 11668019; BAPTA-AM, Sigma Aldrich, A1076; BAPTA, Millipore Sigma 196418; Goat anti mouse IRDye 680 (LI-COR, 925-68020); Goat a Rabbit IRDye 800 (LI-COR, 926-32211); Trueblot anti-mouse DyLight 680, (Rockland, 18-4516-32); Trueblot anti-rabbit DyLight 800 (Rockland, 18-8816-31), BODIPY™ 493/503 (4,4-Difluoro-1,3,5,7,8-Pentamethyl-4-Bora-3a,4a-Diaza-s-Indacene), (ThermoFisher D3922); Oleic Acid-Albumin from bovine serum (Millipore SIGMA 03008); Biotinyl tyramide (biotin-phenol) AdipoGen LIFE SCIENCES CDX-B0270-M100.

### Plasmid Transfections

pDest-GFP-STX17 (Kumar et al., 2018), pDest-GFP-STX17^{S202A}, pDest-GFP-STX17^{S202D} (Kumar et al., 2019), pDest-GFP-VAMP8 (Kumar et al., 2018) FLAG-ATG16L1 (Chauhan et al., 2016), EGFP-DFCP1 (Axe et al., 2008), SIGMAR1-FLAG and SIGMAR1 N80-FLAG (Yang et al., 2019), EGFP-WIPI2b (Bakula et al., 2017) YFP-Parkin (Narendra et al., 2008) have been described earlier. EGFP-hFIP200 (Addgene #38192), mCherry-PIS (Addgene #119078), EGFP-E-SYT2 (Addgene #66831), GFP-VAMP7 (Addgene #45920), were from Addgene. Keima-RPL28 and LDH-Keima was from Heeseon An and J. Wade Harper (Harvard Medical School, Boston, Mass. (An and Harper, 2018). SARS-CoV-2-nsp6 was synthesized and cloned into pDest-GFP or pDest-FLAG. E-SYT2^{6A} was mutated using site directed mutagenesis kit using following primers in GFP-E-SYT2 to generate: (SEQ ID NO. 3) Fw,CGCATGTATTTATTACCAGACGCCGCCGCCTCAGGAGCCGCCGCCACACACGTGTCAAG; (SEQ ID NO: 4) Rw,CTTTGACACGTGTGTGGCGGCGGCTCCTGAGGCGGCGGCGTCTGGTAATAAATACATGCG. Other plasmids and corresponding mutants used in this study, were cloned into pDONR221 using BP cloning, and expression vectors were made utilizing LR cloning (Gateway, ThermoFisher Scientific) in appropriate pDEST vectors for immunoprecipitation and other assays. Point-mutants were generated using the QuikChange Site-directed Mutagenesis Kit (Agilent, 200523). Plasmid constructs were verified by conventional restriction enzyme digestion and/or by DNA-sequencing. Plasmids were transfected using Lipofectamine 2000 (Thermo Fisher Scientific).

### PBMM Assay

HeLa^{WT}, STX17^{KO} and RUBCN^{KO} and Huh7^{WT} and Huh7^{FIP200KO} cells were transfected with Ii-Str-ManII-SBP-EGFP and FLAG-APEX2-TFRC using Lipofectamine 2000 reagent. After transfection, ManII-EGFP was released from hook by giving biotin (40 μM) for 60 min. Cells were then incubated in EBSS or in full media (as indicated in the figures). Samples were then incubated with biotin-phenol (0.5 mM) for 45 min followed by incubation with H₂O₂ for 1 min. Cells were washed three times with quenching buffer (Dulbecco's PBS supplemented with 10 mM sodium ascorbate, 10 mM sodium azide, and 5 mM trolox). Samples were lysed in RIPA lysis buffer. Protein concentrations of lysates were measured, and lysates were incubated with streptavidin beads (Thermo Fisher Scientific) overnight at 4° C. Samples were washed three time with lysis buffer. Proteins were eluted by boiling beads in 2×SDS sample buffer supplemented with 2 mM biotin. Eluted samples and corresponding lysates were subjected to SDS-PAGE followed by western blotting of target proteins.

### High Content Microscopy

HCM was performed as described previously (Kumar et al., 2019). Briefly, cells were plated in 96 well plates and were transfected with plasmids whenever required (as indicated in figures). Cells were stimulated for autophagy by incubating in EBSS for 2 h followed by fixation with 4% paraformaldehyde for 5 mins. Cells were permeabilized with 0.1% saponin and blocked in 3% BSA for 30 mins followed by incubation with primary antibody for 6 h and secondary antibody for 1 h. High content microscopy with automated image acquisition and quantification was carried out using a Cellomics HCS scanner and iDEV software (Thermo) in 96-well plates (Kumar et al., 2019). For HCM experiments >500 primary objects were counted per well, minimum 6 wells were counted per experiments and data presented in figures are derived from at least 3 independent experiments.

### Lipophagy-Lipolysis Assay

HeLaWT or E-SYT2KO cells were plated in 96 well plates. Cells were incubated with oleic acid (500 μM) for 20 h. Cells were then left in full media or incubated with EBSS for 16 h. After completion of treatment times, cells were fixed in 4% PFA followed by two washings with PBS. Cells were then stained with BODIPY (1:500) (ThermoFisher) and Hoechst (1:000) for 15 min followed by three washes with PBS. Finally, lipid droplets were imaged and quantified using HCM as detailed above.

### High Content Microscopy for Keima Probes

293A WT or SIGMAR1^{KO} cells were plated in 96 well plates and transfected with indicated Keima plasmids. Cells were incubated in full media or induced for autophagy by incubating with EBSS for 8 h. Cells were then incubated in full media and incubated with Hoechst 33342 for ten minutes in full media and then acquired for Keima fluorescence at 440 nm and 560 nm using the Cell Insight CX7 High-Content Screening (HCS) Platform (Thermo)(Kumar et al., 2019).
In Vitro Fusion ivitHC Assay
In vitro fusion assay was developed for HCM platform by modifying a previously described assay (Matsui et al., 2018a; Moreau et al., 2011). For in vitro assay, cells were transfected with GFP-FIP200 and mCherry-ATG16L1, post-nuclear supernatants (PNS) were prepared by homogenizing cells in buffer containing 20 mM HEPES-KOH, pH 7.2, 400 mM sucrose, and 1 mM EDTA. Homogenate were centrifuged at 12000 g for 15 min. PNS containing GFP-FIP200 and mCherry-ATG16L1 membranes were mixed for 60 min in the presence of an ATP regenerative system at 37° C. Control samples were left on ice. In experiments when BAPTA was added to the fusion reaction it was used at 20 μM. After the reaction, the samples were fixed with 2% paraformaldehyde in PBS for 15 min, centrifuged to remove the fixative (12,000 g for 15 min), resuspended in mounting media and dispensed in 96 well plates (40 μl/well, at least 5 wells per sample). The plates were centrifuged at 500 g for 1 min to allow settling down of the membranes to bottom of the plate. The plates were scanned in Cell Insight CX7 High-Content Screening (HCS) Platform (Thermo). A minimum of 10,000 objects were scanned per well and 5 wells per sample were used for analysis.

### Super-Resolution Microscopy

Super-resolution imaging, and analysis were done as described previously (Kumar et al., 2019; Kumar et al., 2018). WT or STX17^{KO} HeLa cells were plated on 25 mm coverslips (Warner instruments) and allowed to attach, cells were then transfected with FLAG-ATG16L1. After overnight transfection, cells were induced for autophagy by incubating with EBSS for 2 h followed by fixation in 4% PFA for 5 min. After fixation, cells were incubated with anti-rabbit-FIP200 and anti-mouse FLAG antibodies for 4 h and washed with PBS, followed by labeling with Alexa Fluor 647 (Invitrogen, A21245). The coverslip was mounted on an Attofluor cell chamber (A-7816, life technologies) with 1.1 ml of the imaging buffer. The imaging and center-to-center distances between FLAG-ATG16L1 and FIP200 cluster centroids per ROI (region of interest) were calculated as described earlier (Kumar et al., 2018).

### Immunofluorescence Confocal Microscopy

Immunofluorescence confocal microscopy was carried out as described previously (Kumar et al., 2019). Briefly, cells were plated onto coverslips in 6 well or 12-well plates. Cells were transfected with plasmids as indicated in Figures. Transfected cells were incubated in full media or EBSS (Earle's Balanced Salt Solution) for 2 h and fixed in 4% paraformaldehyde for 10 min followed by permeabilization with 0.1% saponin in 3% BSA. Cells were blocked 3% BSA and then incubated with primary antibodies for 4 h. Cells were washed three times with PBS and then incubated with appropriate secondary antibodies (Invitrogen) for 1 h at room temperature. Coverslips were then mounted using ProLong Gold Antifade Mountant (Invitrogen) and analyzed by confocal microscopy using the Zeiss LSM510 Laser Scanning Microscope.

### Correlative Light-Electron Microscopy

For correlative light-electron microscopy (CLEM), the cells were grown on glass-bottom, gridded Mattek dishes (Cat No P35G-1.5-14-CGRD). Cells were induced for autophagy by incubation in EBSS, followed by fixation in 4% paraformaldehyde in 0.2 M Hepes, pH 7.4 for 10 min at room temperature, and imaged using phase contrast and confocal microscopy to record the cell positions and the fluorescent signals, respectively. The cells were then firmly fixed for electron microscopy using 2% glutaraldehyde in 0.2 M Hepes, pH 7.4, and stored in the same buffer at +4 C. For epon embedding, the cells were postfixed in osmium tetroxide, dehydrated in ethanol, and flat embedded in epon. The Matted grid was used to locate the cells of interest for thin sectioning. Serial 70-nm sections were cut, placed on single-slot girds and stained with uranyl acetate and lead citrate. Imaging was done with a Jeol JEM 1400 Plus transmission electron microscope.
A high magnification TEM micrograph was aligned with a low magnification TEM micrograph, showing the whole cell. The fluorescent image was then correlated to the low magnification TEM micrograph based on the shape of the cell and nucleus as well as lipid droplets that are visible in both TEM and fluorescent images. The alignment and correlation was performed using TrakEM2 software version 1.3.6 (Cardona et al., 2012) as part of the Fiji distribution of ImageJ version 1.53i (Schindelin et al., 2012).

### Membrane Fractionation

Membrane fractionation was carried out as described previously (Kumar et al., 2019). Briefly, 293T cells (3 dishes per sample) were plated in 10 cm dishes, harvested, and homogenized by passing through a 22-G needle. Homogenates were subjected to sequential differential centrifugation at 3,000×g (10 min) and 25,000×g (20 min) to collect the pelleted membranes (TLA100.3 rotor, Beckman, polypropylene tube; Beckman). 25K membrane pellets were suspended in 1 ml 19% OptiPrep for a step gradient containing 0.5 ml 22.5%, 1 ml 19% (sample), 0.9 ml 16%, 0.9 ml 12%, 1 ml 8%, 0.5 ml 5% and 0.2 ml 0% OptiPrep each. The OptiPrep gradient was centrifuged at 150,000×g for 3 h and subsequently eight fractions, 0.5 ml each, were collected from the top. Fractions were diluted with B88 buffer (20 mM HEPES, pH 7.2, 150 mM potassium acetate, 5 mM magnesium acetate, 250 mM sorbitol) and membranes were collected by centrifugation at 100,000×g for 1 h. Sample were subjected to SDS-PAGE and western blot for FIP200, ATG16L1, GM130 and LC3 was done as described under immunoblotting.

### Cholera Toxin B Uptake Assay

Cells were plated in 96 well plates and after suitable transfections (as indicated in figures), cells were incubated with Alexa Fluor-488-conjugated cholera toxin subunit B (ThermoFisher) for 15 min at 4° C. (allowing toxin to bind to the plasma membrane). Then cells were incubated at 37° C. (allowing internalization of cholera toxin) for 10 min and fixed for HCM analysis.

### Protease Protection Assay

E-SYT2^{KO} and wild type HeLa cells, and SIGMAR1^{KO} and parental 293A cells were seeded into 10 cm dishes and induced for autophagy by incubation in EBSS for 2 h in presence of 100 nM bafilomycin A1. After treatment, cells were homogenized in buffer containing 20 mM HEPES-KOH, pH 7.2, 400 mM sucrose, and 1 mM EDTA. Cells were harvested and centrifuged at 500 g at 4° C., the postnuclear supernatant was collected and was equally divided into three parts, one of the samples was left untreated, and the other two were incubated with 25 μg/ml PK only in presence or absence of Triton X-100 (TX-100; 0.2%) for 10 min on ice. All samples were then subjected to TCA precipitation, and protein pellets were resuspended in the same volume of 2× sample buffer. Approximately 40-60 μg of each sample was analyzed by immunoblotting.

### SARS-CoV-2 Infection

Cells were infected with the indicated MOI with SARS-CoV-2, isolate USA-WA/1/2020 (deposited by the Centers for Disease Control and Prevention and obtained through BEI Resources, NIAID, NIH, NR-52281). Cell death was measured at the indicated times using the CyQUANT XTT cell viability assay (ThermoFisher) according to the manufacturer's protocol. For HCM, Calu-3 and Huh7 cells were seeded in 96 well plates and transfected with FLAG-ATG16L1 and then taken to BSL3 facility. Half of the plate was incubated in EBSS (2 h). Cells were then infected with SARS-CoV-2 (MOI:1) for 24 h. Cells were then fixed with 4% paraformaldehyde for 15 min. Plates were decontaminated, permeabilized with 0.2% saponin and blocked with 3% BSA for 30 min and stained with FLAG and FIP200 antibodies. HCM was performed as detailed under HCM section.

### APEX2-Labeling and Streptavidin Enrichment for Mass Spectrometry

HEK293T Flp-In-nsp6^{TetON} cells were left in full media or incubated with EBSS for 2 h. Cells were then incubated in 500 μM biotin-phenol for the last 30 min of EBSS incubation. Cells were then incubated in 1 mM H₂O₂ at room temperature. The reaction was stopped with quenching buffer (10 mM sodium ascorbate, 10 mM sodium azide and 5 mM Trolox in Dulbecco's Phosphate Buffered Saline [DPBS]). All samples were washed three times with quenching buffer, and twice with DPBS. For LC-MS/MS analysis, cell pellets were lysed in 500 μL ice-cold lysis buffer (6 M urea, 0.3 M NaCl, 1 mM EDTA, 1 mM EGTA, 10 mM sodium ascorbate, 10 mM sodium azide, 5 mM Trolox, 1% glycerol and 25 mm Tris/HCl [PH 7.5]) for 30 min by gentle pipetting. Lysates were clarified by centrifugation and protein concentrations were quantified. Streptavidin-coated magnetic beads (Pierce) were washed with lysis buffer. 3 mg of each sample was mixed with 100 μL of streptavidin bead. The suspensions were gently rotated at 4° C. overnight to bind biotinylated proteins. The flowthrough after enrichment was removed and the beads were washed in sequence with 1 mL IP buffer (150 mM NaCl, 10 mM Tris-HCl [pH 8.0], 1 mM EDTA, 1 mM EGTA, 1% Triton X-100) twice; 1 mL 1M KCl; 1 mL of 50 mM Na₂CO₃; 1 mL 2 M Urea in 20 mM Tris HCl (pH 8); 1 mL IP buffer. Biotinylated proteins were eluted and processed for mass spectrometry.

### LC-MS/MS (Sample Preparation)

Protein samples on magnetic beads were washed four times with 200 ul of 50 mM Triethyl ammonium bicarbonate (TEAB) with a twenty-minute shake time at 4° C. in between each wash. Roughly 2.5 μg of trypsin was added to the bead and TEAB mixture and the samples were digested overnight at 800 rpm shake speed. After overnight digestion the supernatant was removed, and the beads were washed once with 50 mM ammonium bicarbonate. After 20 minutes at a gentle shake the wash is removed and combined with the initial supernatant. The peptide extracts were reduced in volume by vacuum centrifugation and a small portion of the extract was used for fluorometric peptide quantification (Thermo scientific Pierce). One microgram of sample based on the fluorometric peptide assay was loaded for each LC-MS analysis.

### Liquid Chromatography Tandem Mass Spectrometry

Peptides were analyzed by LC-MS/MS by trapping on a C18 peptide trapping column (Thermo Scientific) and separated on a Pepsep 8 cm×150 um C18 column (Merslev, Denmark) using a Dionex Ultimate 3000 nUPLC. Samples were run on Thermo Scientific Exploris 480 mass spectrometer for 90 min using Data independent Acquisition mode (DIA). DIA was acquired between 360-1200 Da with 45 Da non-overlapping windows. MS resolution was set at 120K and MS2 at 30K, MS1 normalized AGC target of 300% and MS2 1000% respectively.

### Data Analysis

DIA data was analyzed using Spectronaut 15 with directDIA workflow and default settings.

### Flow Cytometry to Analyze Intracellular Calcium

Intracellular calcium was analyzed using FLUO-3AM fluorescence on the FL-1 channel of flow cytometer (BD FACScan). Wild type HeLa or STX17Ko cells were left unstimulated on incubated in HBSS for 2 h. Cells were incubated with 5 μM of FLUO-3AM for 30 minutes, followed by analysis on flow cytometer.

### Immunoblotting and Co-Immunoprecipitation Assays

Immunoblotting and co-immunoprecipitation (co-IP) were performed as described previously (Kumar et al., 2018). For co-IP, cells were transfected with 10 μg of plasmids, wherever stated, and lysed in NP-40 buffer containing protease inhibitor cocktail (Roche, cat #11697498001) and PMSF (Sigma, cat #93482). Lysates were mixed with 5 μg antibody and incubated at 4° C. for overnight followed by incubation with Dynabeads protein G (Life Technologies) for 4 h, at 4° C. Beads were washed three times with PBS and then boiled with SDS-PAGE buffer for analysis of interacting protein by immunoblotting.

### Quantification and Statistical Analysis

Data are expressed as means±SEM (n≥3). Data were analyzed with a paired two-tailed Student's t-test or analysis of variance (ANOVA). Statistical significance was defined as † p≥0.05, *P<0.05, **p<0.01.

### Results

### FIP200 and ATG16L1 Compartments Merge During Autophagy

To test whether FIP200 and ATG16L1 compartments fuse during autophagy induction, we employed a panel of techniques. Confocal microscopy and high content microscopy (HCM) quantification showed overlaps between FIP200 and ATG16L1 compartments, which increased upon autophagy induction by starvation (EBSS) using antibodies against endogenous proteins (FIG. 1A,B). Similar results were observed with FIP200 and FLAG-ATG16L1 profiles in HeLa cells (FIGS. 1C and S1A) and in primary human bronchial epithelial cells (Figure S1B,C).

In full medium, FIP200 displayed mostly perinuclear Golgi localization (Figure S1A) (Kumar et al., 2019) and localized with the cis-Golgi marker GM130 (Figure S1D-F). Upon starvation-induced autophagy FIP200⁺GM130⁺ puncta did not increase in abundance (Figure S1E,F) but dispersed from their usual perinuclear location of the Golgi (Figure S1G-I) increasing the number of punctate FIP200 profiles (Figure S1J,K).

ATG16L1 profiles increased in cells induced for autophagy (Figure S1L,M), with a subset of FLAG-ATG16L1 colocalizing with both FIP200 and GM130 (Figure S1N). The % of FIP200 profiles positive for FLAG-ATG16L1 increased upon starvation-induced autophagy (Figure S1A) as quantified by HCM (FIGS. 1C, S1O). This was confirmed with endogenous FIP200 and ATG16L1 (Figure S1 P,Q), and in another cell line, Huh7 (Figures S1R,S), indicating merger of FIP200 and ATG16L1 profiles during autophagy induction.

By CLEM ultrastructural analysis, this compartment appeared as a combination of vesicular and cisternal profiles, observed most distinctly in the vicinity of both lipid droplets (FIG. 1D, subpanels i-iii) and unclosed autophagosomes (FIG. 1D, subpanels iv-vii). In EM serial sections, the yellow fluorescence surrounding lipid droplets (corresponding to GFP-FIP200 and mCherry-ATG16L1), appeared morphologically as groups of vesicular (40-55 nm in diameter; FIG. 1D, white arrows) and cisternal (FIG. 1D, gray arrows) structures of varying sizes and sometimes bent shapes. The morphologically distinct phagophore in FIG. 1D (subpanels iv-vii; traced in vii) overlapped with the yellow fluorescence corresponding to the phagophore membrane and was on a cradle of ER in the vicinity of a mitochondrion with potential ER-mitochondrion contact. We interpret the CLEM results as an indication that FIP200-ATG16L1 compartments coincide with vesicular and cisternal profiles in the vicinity of membranous profiles surrounding lipid droplets or are parts of standalone nascent phagophores.

Fluorescently labeled cholera toxin B (CtxB) has been used as a probe to define the ATG16L1⁺ endosomal compartments derived from PM that participate in autophagosome formation (Ravikumar et al., 2010). We used CtxB-Alexa Fluor 488 (CtxB-488) to detect mixing of cis-Golgi-derived FIP200 and endosomal ATG16L1 compartments. As with ATG16L1 puncta (Figure S1L,M), CtxB profiles increased during starvation (Figure S1T,U). The number of CtxB-488⁺ profiles that were simultaneously positive for FIP200 and ATG16L1 increased upon autophagy induction (Figures S1V-X) paralleled by increases in % of CtxB-488⁺ profiles positive for FIP200 (Figure S1Y,Z) or FLAG-ATG16L1 (Figure S1A¹,B¹). Several endosomal sub-compartments contribute to ATG16L1 autophagosome precursors, including ATG9A vesicles (Moreau et al., 2011; Puri et al., 2013). ATG9A^{KO} cells showed increased basal colocalization between FIP200 and FLAG-ATG16L1, but no further increase upon starvation (Figure S1C¹,D¹), suggesting that ATG9A affects the flow of membranes to these compartments. In summary, cis-Golgi-derived FIP200 and ATG16L1 originating from PM-derived endosomes, merge during autophagy.

### STX17 is Required for a Merger Between FIP200 and ATG16L1 Compartments During Autophagy Induction

STX17 is a SNARE first thought to play a role in autophagosome-lysosome fusion (Itakura et al., 2012) but other studies (Arasaki et al., 2018; Hamasaki et al., 2013; Kumar et al., 2019; Sugo et al., 2018) suggest that STX17 may act earlier in the pathway around initiation stages, consistent with STX17-ATG14L interactions (Diao et al., 2015) and ATG14L's function during initiation (Baskaran et al., 2014; Mizushima et al., 2011). STX17 influences FIP200-containing initiation complexes (Kumar et al., 2019). We tested whether STX17 affects the merger of FIP200 and ATG16L1 compartments by quantifying colocalization of FIP200 and FLAG-ATG16L1 profiles in STX17 KO (STX17^{HeLaKO}) (Kumar et al., 2018) vs STX17^{HeLaWT} cells. STX17 was required for fusion of FIP200 and FLAG-ATG16L1 compartments (FIGS. 1E,F and S2A,B). An siRNA knockdown of STX17 reduced overlap between FLAG-ATG16L1 and FIP200, a phenotype that was complemented by siRNA-insensitive mouse Stx17 (Figure S2 C,D). GFP-STX17 colocalization with FLAG-ATG16L1 and FIP200 increased upon autophagy induction (Figure S2 E,F). In STX17^{HeLaKO} cells, the area of FLAG-ATG16L1 punctate profiles increased upon autophagy induction (Figure S2G,H), reflecting precursor accumulation. Since STX17 is required for FIP200 peripheral profiles (Kumar et al., 2019), the number of FIP200 puncta was reduced in STX17^{HeLaKO} cells. ATG16L1 profiles increased in positivity for the endosomal marker transferrin receptor (TFRC) (Figure S2I,J). The defect in fusion was confirmed in a different cell line (Huh7) (Gu et al., 2019): STX17^{Huh7KO} showed no response to starvation whereas in STX17^{Huh7WT} cells the % of FIP200 ATG16L1⁺ profiles increased (FIGS. 1G and S2K). Super-resolution microscopy (FIG. 1H,I) and cluster quantification showed that FIP200 and FLAG-ATG16L1 were dispersed in STX17^{HeLaKO} vs STX17^{HeLaWT} cells (FIG. 1I). Thus, STX17 is a SNARE contributing to the merger of FIP200 and ATG16L1 compartments.

### FIP200 and ATG16L1 Compartments Undergo Membrane Fusion

One hypothesis that emerged from the above studies is that FIP200 and ATG16L1 represent two precursor membranes with STX17 facilitating heterotypic fusion between them early in autophagy. To test this, we used an established assay for in vitro membrane fusion (Matsui et al., 2018a; Moreau et al., 2011; Puri et al., 2013; Wang et al., 2017). This method mixes vesicles from different cells expressing green (GFP-FIP200) and red (mCherry-ATG16L1) markers and after addition of an ATP and ATP-regenerating system vesicle fusion is monitored by fluorescence microscopy. We adapted this to permit HCM quantification allowing highly powered analyses and termed it IvitHC (in vitro fusion high content assay) (FIG. 2A). ATP-dependent fusion between GFP-FIP200 and mCherry-ATG16L1 vesicles peaked 1 h after autophagy induction (Figure S2L,M), resulting in an 8-fold merger of GFP-FIP200 and mCherry-ATG16L1 in wt cells, whereas in STX17^{HeLaKO} fusion was diminished (FIG. 2B,C). Thus, STX17 is required for formation of a hybrid pre-autophagosomal structure (HyPAS) observed both in vivo and in vitro.

To confirm that membrane fusion occurs, we developed an assay for content mixing between the two compartments termed proximity-biotinylation membrane-content mixing assay (PBMM) (FIG. 2D). PBMM consists of an integral membrane protein target in one (‘donor’) compartment and APEX2-fused to a different membrane protein in the partner (‘acceptor’) compartment (FIG. 2D). If the two membranous compartments fuse, either protein can diffuse through the delimiting membrane of the hybrid structure, and proximity biotinylation can occur. If the membranes are just tethered but not fused, the two membranes stay separated and APEX2-has no or very limited access to the target protein. To provide additional layer of control, we combined PBMM with the RUSH system (Boncompain et al., 2012), which is based on a ‘hook’ and a ‘reporter’ (FIG. 2D). The ‘hook’ acts as a clamp and keeps the ‘reporter” transmembrane protein in the ER (based on streptavidin-streptavidin binding protein/SBP cassette integrated into the transmembrane protein). Only upon breaking the hook-reporter interaction, which is sensitive to biotin, is the reporter free to traffic to its normal intracellular location. We employed the characterized ManII-reporter and an ER hook, which allowed us to control location of ManII in the cell: only after adding biotin did ManII relocate to cis-Golgi (Figure S2N) and colocalized with FIP200 (Figure S2O).

The ‘acceptor’ compartment in the PBMM system consisted of APEX2-TFRC, with APEX2 facing the cytosol. Target biotinylation (ManII) can occur only after the two compartments fuse allowing ManII and TFRC to come in proximity by diffusion. ManII was biotinylated (assessed by enrichment on avidin beads followed by immunoblotting) in cells co-expressing APEX2-TFRC only after release of the RUSH clamp and was enhanced by starvation, but only in STX17^{WT} and not STX17^{KO} cells (FIG. 2E,F). A knockout of FIP200 in HeLa cells reduced starvation-induced mixing of the two compartments (FIG. 2G,H). For comparison, we tested the effects of RUBCN. RUBCN is required for LC3 lipidation on noncanonical single membrane structures but is not required for double membrane canonical autophagy (Martinez et al., 2015). Knocking out RUBCN affected neither starvation-induced membrane mixing (Figure S2P) nor HyPAS formation (Figure S2Q,R). Thus, the starvation-induced mixing between cis/early-Golgi compartments (ManII) and endosomal compartments (TFRC) required FIP200, a component of the canonical autophagy core complex transducing signals from mTOR and AMPK upon starvation-induced autophagy.

### Relationship of HyPAS to Other Steps of the Autophagy Pathway

We tested whether HyPAS is on the pathway for downstream ATG-dependent processes of canonical autophagy or noncanonical pathways (Galluzzi and Green, 2019) such as LC3-associated phagocytosis (LAP) (Martinez et al., 2015; Sanjuan et al., 2007) and LAP-like processes (Fletcher et al., 2018; Heckmann et al., 2019; Jacquin et al., 2017). Comparing STX17^{HeLaKO} and STX17^{HeLaWT} cells, we detected no effects of STX17 KO on increase in LC3-ATG16L1 overlap, as a measure of LAP-like noncanonical autophagy (Fletcher et al., 2018; Jacquin et al., 2017) in response to monensin (Figure S2S,T).

We next used early markers of autophagy initiation, phosphatidylinositol synthase (PIS) (Nishimura et al., 2017) and DFCP1 (Axe et al., 2008). STX17 KO prevented starvation-induced greater colocalization between endogenous FIP200 and mCherry-PIS (Figure S3 A-C). DFCP1 marks omegasomes, membranous structures found in the proximity of LC3 profiles believed to act as a cradle for nascent autophagosomes (Axe et al., 2008; Tooze and Yoshimori, 2010). DFCP1 kinetically segregated from HyPAS and appeared later than HyPAS (Fig S3D,E). HyPAS formation preceded WIPI2b (Dooley et al., 2014) but eventually became positive for WIPI2b (Figure S3F,G). HyPAS appearance preceded LC3⁺ stage by 15 min (Figure S3H,I) with delayed recruitment of LC3 to HyPAS 30 min after autophagy induction. This makes HyPAS a prophagophore. Double positive LC3⁺ FLAG-ATG16L1⁺ profiles were reduced in STX17^{KO} cells (Figure S3J-L). HyPAS was independent of the six mATG8s inactivated in Hexa^{KO} cells (Nguyen et al., 2016) (LC3A, LC3B, LC3C, GABARAP, GABARAPL1 and GABARAPL2) which showed normal HyPAS formation (Figure S3M,N). We conclude that HyPAS formation through STX17-dependent heterotypic fusion of FIP200 and ATG16L1 vesicles affects other known events during autophagy initiation.

### FIP200-ATG16L1 Complexes Depend on STX17

FIP200 and ATG16L1 form protein complexes (Fujita et al., 2013; Gammoh et al., 2013; Nishimura et al., 2013). Does formation of these complexes need fusion of precursor membranes? When we compared the ability of endogenous FIP200 and FLAG-tagged ATG16L1 to form protein complexes in HeLa^{WT} vs. STX17^{KO} cells, STX17 was absolutely required (FIG. 3A,B) confirmed with endogenous proteins (Figure S3O).

We examined the distribution of endogenous ATG16L1 and FIP200 by cell membrane fractionation. In cells induced for autophagy by starvation, a membranous compartment with ATG16L1 and FIP200 was formed (HyPAS) (Figure S3P, left panel). At least a portion of this compartment contained LC3-II, a downstream autophagosomal marker. HyPAS membranes were enhanced in intensity in cells induced for autophagy (EBSS) relative to fed cells (Figure S3P). This compartment did not form in STX17^{KO} cells (Figure S3Q, right). Instead, in STX17^{KO} cells ATG16L1 and FIP200 floated in a very light membranous neo-compartment, in addition to FIP200 being retained in heavy fractions together with the Golgi marker GM130 (Figure S3Q).

### SNARE Complexes Controlling HyPAS Formation

Proteomic analyses with STX17 (Kumar et al., 2019) revealed two R-SNAREs, VAMP7 and VAMP8 as STX17 interactors (FIG. 3C), in keeping with prior work (Wang et al., 2016). VAMP8 is implicated in autophagosome-lysosome fusion (Itakura et al., 2012). VAMP7's role is less clear albeit it is implicated in autophagy initiation via ATG16L1 vesicles (Moreau et al., 2011). Under basal conditions, VAMP7 associated with ATG16L1 but not with FIP200 in Co-IPs whereas STX17 associated with FIP200 (FIG. 3D,E). In cells induced for autophagy, FLAG-STX17 and endogenous VAMP7 colocalized (Figure S3R). VAMP7 colocalized with HyPAS in starved cells (Figure S3S). Knocking down (KD) VAMP7 reduced the formation of HyPAS (FIGS. 3F,G and S3 T,U) while KD of VAMP8 did not (Figure S4A,B). We tested in vitro the effects of VAMP7 on HyPAS formation by IvitHC and found reduced HyPAS formation using vesicles from VAMP7 KD cells (FIG. 3H-J). Thus, VAMP7 contributes to early stages of autophagosome formation via fusion of FIP200⁺ and ATG16L1⁺ precursor membranes.

Proteomic analyses with STX17 (Figure S4C) showed that it interacts with the Q_{bc} SNARE SNAP-47 in addition to its well characterized Q_{bc} partner SNAP-29 (Diao et al., 2015; Itakura et al., 2012; Takats et al., 2013; Wang et al., 2016). GFP-SNAP-47 colocalized with FLAG-STX17 (Figure S4D). SNAP-47 co-IPed with STX17 but not with STX17 lacking its SNARE domain (Figure S4E,F). KD of SNAP-29 did not affect HyPAS formation (Figure S4G), suggesting that this Q_{bc} SNARE, which acts in autophagosome-lysosome fusion (Diao et al., 2015; Itakura et al., 2012; Takats et al., 2013; Wang et al., 2016) does not participate in HyPAS formation. We could not achieve KD of SNAP-47, to rule in or out this particular SNARE.

### Phosphorylation of STX17 Directs R-SNARE Partnering During HyPAS Formation

STX17 is phosphorylated by TBK1 at S202 and this is important in formation of mammalian pre-autophagosomal structures (Kumar et al., 2019). We tested whether TBK1 affected HyPAS formation. TBK1^{KO} HeLa cells (Kumar et al., 2019) had reduced HyPAS formation (FIG. 3 K,L). VAMP7 but not VAMP8 lost binding to a non-phosphorylatable mutant (S202A) of STX17 (Figures S4H-K). Complementation of STX17^{KO} cells with WT STX17 or with its phosphomimetic mutant STX17^{S202D} rescued HyPAS formation, whereas the non-phosphorylatable STX17^{S202D} mutant did not (FIGS. 3M and S4L). Thus, phosphorylation of STX17 and its kinase TBK1 regulate the specificity of STX17 interactions with its R-SNARE partners during HyPAS formation.

### SERCA2 is an STX17 Partner and Ca²⁺ Affects HyPAS Formation

Mining the mass spectrometry database MS (MSV000083251) deposited for proteomic analyses with GFP-STX17 (Kumar et al., 2019) uncovered that one of STX17's binding partners is the ubiquitous ER calcium pump SERCA2 (FIG. 4A) participating in the early stages of autophagosomal biogenesis (Zhao et al., 2017). The interaction between STX17 and SERCA2 was observed with endogenous proteins (FIG. 4B) and confirmed in co-IPs between GFP-STX17 and SERCA2 (Figure S4M). FLAG-STX17 colocalized with endogenous SERCA2 (Figure S4N). SERCA2 pumping removes cytosolic Ca²⁺ (MacLennan and Kranias, 2003; Periasamy and Kalyanasundaram, 2007; Vandecaetsbeek et al., 2011), and conceivably STX17-SERCA2 interactions could involve Ca²⁺. Thus, we tested whether chelating cytoplasmic Ca² with BAPTA-AM affected HyPAS formation. Under starvation-induced autophagy, HyPAS formation was reduced by BAPTA-AM to an extent similar to the effects of STX17 KO, whereas BAPTA-AM did not further affect HyPAS formation in STX17^{KO} cells (FIGS. 4C,D and S4O,P). A similar effect of chelation by BAPTA was observed by IvitHC (FIG. 4E,F). SERCA2 is inhibited by thapsigargin (TG), and so the expected effect of TG would be increased HyPAS formation. In cells treated with TG (Engedal et al., 2013; Ganley et al., 2011), HyPAS formation was increased in basal conditions (FIG. 4 G). The effect of TG was abrogated in STX17^{KO} cells (FIGS. 4 G,H and S4Q,R). SERCA2 expression inhibited HyPAS (FIG. 4I). SERCA2 was present in multiple fractions on OptiPrep gradients (Figure S3P). During autophagy induction (HBSS), cytosolic Ca²⁺ (measured by FLUO-3) was elevated (FIG. 4J,K), as described (Cardenas et al., 2010). In STX17^{KO} cells, cytosolic calcium did not increase, consistent with unabated activity of SERCA2 in the absence of its partner STX17 (FIG. 4J,K). We conclude that STX17 interacts with SERCA2 and that STX17 directly or indirectly inhibits its function (FIG. 4L) during autophagy induction.

### E-SYT2, an STX17 Interactor, Affects HyPAS Formation

Among other potential interactors of STX17 based on the MS (MSV000083251) database we identified E-SYT2 as an STX17 partner (FIG. 5A). E-SYT2 is a member of the ESYT family of ER proteins with a role for Ca²⁺ in tethering to PM (Giordano et al., 2013, Min et al., 2007. Saheki et al., 2016). Interactions between STX17 and E-SYT2 were observed with endogenous proteins (FIG. 5B) and confirmed in co-IPs (Figure S5A). FLAG-STX17 and GFP-E-SYT2 profiles overlapped by immunofluorescence (FIG. 5C). FLAG-E-SYT2 colocalized with HyPAS (Figure S5B). E-SYT2-KD prevented HyPAS formation (Figure S5C-E) indicative of fusion arrest by the juxtaposition of ATG16L1 and FIP200 profiles (Figure S5C) similar to the STX17 KO effects (FIG. 1E).

E-SYT2^{KO} cells (Saheki et al., 2016) displayed juxtaposition of ATG16L1 and FIP200 profiles (FIGS. 5D and S5F), with no HyPAS formation (FIG. 5E,F) upon autophagy induction (EBSS). E-SYT2 was necessary for in vitro HyPAS formation by IvitHC (FIG. 5G,H). E-SYT2 was required for mixing of cis-Golgi and endosomal compartments in the PBMM assay (FIG. 5 I,J). Finally, E-SYT2 was important for HyPAS formation in response to SERCA inhibition by TG (Figure S5G,H), suggesting that it acts as a downstream effector of SERCA and Ca²⁺. Thus, in addition to SERCA, which regulates cytosolic Ca²⁺, E-SYT2, which can act as an effector of Ca²⁺ (Giordano et al., 2013; Saheki et al., 2016), controls HyPAS formation.

E-SYTs play a role in membrane tethering at ER-PM contact sites (Giordano et al., 2013; Saheki et al., 2016). Synaptotagmins in principle control SNARE complexes with the paradigm being based on Synaptotagmin 1 (SYT1) (Brose et al., 1992; Chapman, 2002). We thus wondered if E-SYT2 may have an as-yet unrecognized function in HyPAS formation catalyzed by SNAREs. Homology alignments with SYT1 revealed conserved polybasic regions in E-SYT2's C2C domain corresponding to the polybasic region in SYT1's C2B domain (FIG. 5K). We mutated six basic residues in E-SYT2, K805, R806, R807, R809, R810, K811A to generate E-SYT2^{6A}. The corresponding polybasic stretch in SYT1 is essential for the regulatory effects of SYT1 on its cognate syntaxin (Brewer et al., 2015). Co-IP analyses showed reduced binding to STX17 of E-SYT2^{6A} vs GFP-E-SYT2^{WT} (FIG. 5L,M). E-SYT2^{6A} could not rescue HyPAS formation in E-SYT2^{KO} whereas E-SYT2^{WT} did (FIGS. 5N and S5I). Thus mutational analysis, based on prototypical synaptotagmin-syntaxin relationships, revealed that E-SYT2 interacts with STX17 to regulate its function in HyPAS formation.

We observed reduced levels of STX17 in VAMP7 immunoprecipitates from E-SYT2^{KO} cells (FIG. 5O,P). Comparing overexpressed GFP-VAMP7 and GFP-VAMP8 in complexes with FLAG-STX17, we observed that in E-SYT2^{KO} cells GFP-VAMP7, but not GFP-VAMP8, was diminished in SNARE complexes (FIG. 5Q,R). Thus E-SYT2, in addition to its previously characterized roles in lipid transfer (Saheki et al., 2016) and PI3P production at ER-PM contact sites during peripheral LC3 puncta formation (Nascimbeni et al., 2017), plays a role in early stages of autophagosome formation.

### HyPAS Depends on SIGMAR1

The STX17 interactors SERCA and STX17 controlling HyPAS formation are located in the ER. Another ER-localized interactor of STX17 is SIGMAR1 (Yang et al., 2019), which plays a role in autophagy (Christ et al., 2019; Vollrath et al., 2014; Yang et al., 2019). SIGMAR1 co-fractionated with HyPAS in OptiPrep density gradients (Figure S3O). HyPAS formation was reduced in SIGMAR1^{KO} cells (Yang et al., 2019) relative to WT cells (FIG. 6A-C). FLAG-SIGMAR1 co-IPed with VAMP7 (Figure S5J). Complementation, of SIGMAR1^{KO} with full length FLAG-SIGMAR1 recovered HyPAS formation (FIGS. 6D,E and S5K). Expression of a truncated version of SIGMAR1, FLAG-SIGMAR1N80 (Yang et al., 2019), consisting of the N-terminal transmembrane domain and only a portion of the cytosolic-facing surface of the SIGMAR1 protomers within the SIGMAR1 trimeric architecture (Schmidt et al., 2016), did not restore HyPAS formation (FIGS. 6D,E and S5K). This was in keeping with effects on mitophagy (Yang et al., 2019). Thus, the ability of SIGMAR1 to form trimers or to interact with additional components is necessary for HyPAS formation in autophagy.

### HyPAS is Important for Conventional Autophagy of Diverse Cargo

Next tested was whether E-SYT2 and, by extension, HyPAS are responsible for removal of autophagic cargo. We tested Parkin-dependent mitophagy (Narendra et al., 2008; Youle, 2019) in E-SYT2^{WT} and E-SYT2^{KO} HeLa cells transfected with YFP-Parkin (Narendra et al., 2008), using mtDNA antibody to quantify mitophagy (Gu et al., 2019; Lazarou et al., 2015; Nguyen et al., 2016) by HCM while gating on YFP-Parkin⁺ cells. E-SYT2^{KO} cells had diminished mitophagy relative to WT elicited with OA (oligomycin+antimycin) (FIG. 6F,G) or CCCP (Figure S5L,M). In a complementation assay, E-SYT2^{6A} mutant, which does not bind STX17, failed to restore mitophagy whereas E-SYT2^{WT} did (FIGS. 6H and S6A). SIGMAR1^{KO} cells transfected with YFP-Parkin showed reduced mitophagy in response to CCCP (Figure S6 B,C). Thus, HyPAS is required for mitophagy.

We next tested autophagy of ribosomes (ribophagy) (An and Harper, 2018; An et al., 2020; Eskelinen, 2008; Tanaka et al., 2000; Wyant et al., 2018). We transfected the RPL28-Keima ribophagy probe (An and Harper, 2018)) into WT and E-SYT2^{KO} HeLa cells. After starvation for 8 h in EBSS, Keima-positive autolysosomal organelles engaged in ribophagy were quantified by HCM (Ex/Em 560/620 nm) (Katayama et al., 2011; Violot et al., 2009). E-SYT2^{KO} cells showed reduced ribophagy relative to E-SYT2^{WT} parental cells (Figure S6D,E). The E-SYT2^{6A} mutant failed to complement ribophagy whereas E-SYT2w did (Figure S6F,G). SIGMAR1^{KO} 293A cells exhibited reduced ribophagy (FIGS. 6I and S6H). Thus, HyPAS is important for ribophagy.

We next tested bulk autophagy (Kopitz et al., 1990; Pattingre et al., 2003; Szalai et al., 2015) employing LDH-Keima construct (An and Harper, 2018). E-SYT2^{KO} cells had fewer autolysosomes containing the bulk autophagy probe LDH-Keima (An and Harper, 2018) relative to E-SYT2^{WT} cells (FIGS. 6J and S6I), and confirmed in SIGMAR1^{KO} 293A cells (FIGS. 6K and S6 J).

In response to starvation, E-SYT2KO cells exhibited reduced lipophagy-lipolysis (Zechner et al., 2017) (Figure S6K,L) and degradation of endogenous SQSTM1/p62 (Figure S6M,N), a principal autophagy receptor (Bjorkoy et al., 2005; Pankiv et al., 2007). The starvation-induced degradation of p62 paralleled that of ManII (Figure S6O,P), an early Golgi resident enzyme that colocalizes with FIP200 and was utilized in the PBMM assay (Figure S2O). ManII was degraded during starvation, abrogated in E-SYT2^{KO} cells (Figure S6O,P). Thus, HyPAS is critical for autophagy of diverse cargo.

Phagophores eventually close to sequester cargo. E-SYT2 was required for protection of p62 from proteinase K (Nguyen et al., 2016; Velikkakath et al., 2012), an assay for autophagic sequestration (Figure S6Q,R). ESCRTs catalyze phagophore closure (Takahashi et al., 2018; Zhen et al., 2020). When cells are depleted of the ESCRT-III component CHMP2A, this results in morphologically scorable aberrant retention of the ESCRT-III component CHMP4B (Teis et al., 2008) on unclosed autophagosomes (Zhen et al., 2020). In cells induced for autophagy by starvation, CHMP4B puncta increased upon CHMP2A KD, indicative of accumulating unclosed autophagosomes (Zhen et al., 2020). The CHMP4B puncta were diminished in E-SYT2^{KO} HeLa cells, indicative of fewer phagophores being formed (Fig. S6 S,T).

### HyPAS is a Target of Pharmacological Agents

SIGMAR1 is a target of pharmacological agents (Christ et al., 2019; Hayashi and Su, 2007; Hirata et al., 2011; Vollrath et al., 2014) including chloroquine (CQ) (Gordon et al., 2020; Schmidt et al., 2016), an inhibitor of autophagy (Klionsky et al., 2016). CQ neutralizes lysosomes (Klionsky et al., 2016), perturbs Golgi (Mauthe et al., 2018) and binds SIGMAR1 (Gordon et al., 2020; Schmidt et al., 2016). In the IvitHC assay, CQ inhibited HyPAS formation in vitro (Figure S7A,B) while bafilomycin A1 did not (Figure S7A,B). The known CQ target SIGMAR1 was important for STX17 and E-SYT2 interactions (Figure S7C,D) whereas CQ treatment inhibited them (Figure S7E, F). The effect was specific since CQ did not inhibit STX17 and SERCA2 interactions (Figure S7E,G). Thus, CQ interferes with autophagy at a very early point, HyPAS formation.

We next tested other SIGMAR1 ligands implicated in autophagy (Christ et al., 2019; Hirata et al., 2011; Maher et al., 2018; Tesei et al., 2018) for their effects on HyPAS. The agonist cutamesine induced HyPAS formation in cells grown in full medium (Figure S7H,I) whereas the antagonist BD1047 inhibited HyPAS formation induced by starvation (Figure S7J,K). Thus, pharmacological agonists and antagonists of SIGMAR1 affect the formation of the precursor structure to mammalian autophagosomes.

### SARS-CoV-2 Infection and SARS-CoV-2 Nsp6 Target and Inhibit HyPAS

Autophagy intersects morphologically with coronavirus biogenesis (Cottam et al., 2011; Cottam et al., 2014; Fung and Liu, 2019, Gassen et al., 2019; Hoffmann et al., 2021; Miao et al., 2021; Reggiori et al., 2010; Schneider et al., 2021). We tested whether HyPAS was targeted during infection employing Huh7 and additionally Calu3 cells, a human cell line used to study SARS-CoV-2 infection in disease site-relevant context (Hoffmann et al., 2020a; Hoffmann et al., 2020b). Cells were infected with SARS-CoV-2 (USA-WA1/2020) using viral preparations causing cytopathic effect in Vero E6 (Bradfute et al., 2020) and verified in Huh7 cells (Figure S7L). SARS-CoV-2 infection inhibited HyPAS formation in Calu3 (FIGS. 7A,B) and Huh7 (Figures S7M,N) cells. Thus, HyPAS is affected by SARS-CoV-2.

SARS-CoV Nsp6 causes LC3 puncta to be smaller than regular autophagosomes (Cottam et al., 2014). The SARS-CoV-2 ORF1 polyprotein, which includes nsp6, shows 76% identity to SARS-CoV (Zhou et al., 2020). We carried out proximity biotinylation proteomic analysis with APEX2-SARS-CoV-2-nsp6 in stably transfected (FLIP-IN) cells with APEX2-nsp6 expression controlled by the Tet-ON system (FIG. 7C, Table ST1A-C). The functionality of the SARS-CoV2-nsp6 construct was validated in an assay developed for nsp6 of SARS-CoV (Cottam et al., 2014) (Figure S7O). The proteomic data confirmed the previously reported nsp6-SIGMAR1 interaction (Gordon et al., 2020) and revealed that nsp6 interacts with VAMP7, E-SYT2, SERCA2, and TBK1 (FIG. 7D, Table ST1B). Interactions between SARS-CoV-2 nsp6 and SERCA2 were validated by co-1P (Figure S7P). In summary, proximity biotinylation proteomic analysis uncovered ESYT2, VAMP7, and SERCA2 as SARS-CoV-2 nsp6 interactors, and revealed that the HyPAS regulator TBK1, which phosphorylates STX17 (Kumar et al., 2019) and authorizes it to engage VAMP7, is targeted by SARS-CoV-2 nsp6.

We next tested whether SARS-CoV-2 nsp6 affects HyPAS. Following published procedures (Cottam et al., 2014), we expressed SARS-CoV-2 nsp6 in HeLa cells and quantified HyPAS formation by HCM. SARS-CoV-2 nsp6 (GFP-nsp6) reduced HyPAS yields upon autophagy induction (EBSS) (FIGS. 7E and S7Q,R). In contrast to nsp6, expression of SARS-CoV-2 ORF3a, reported to interfere with autophagosomal fusion with lysosomes (Miao et al., 2021), or ORF8 did not significantly inhibit HyPAS formation (FIGS. 7F,G and S7S,T).

To test the effects of nsp6 in vitro by IvitHC, we co-expressed FLAG-nsp6 with GFP-FIP200 and separately co-expressed FLAG-nsp6 with mCherry-ATG16L1 in HeLa cells before preparing vesicles for in vitro fusion (FIG. 7H). IvitHC showed that nsp6 inhibited HyPAS formation in vitro (FIG. 7I,J). Thus, SARS-CoV-2 nsp6 interferes with early formation of autophagosomes at the HyPAS stage.

### Discussion

The inventors have identified a critical step in the biogenesis of canonical autophagosomes in mammalian cells. This step is embodied in HyPAS, a prophagophore compartment formed through fusion of membranes derived from the constitutive secretory pathway and the endosomal pathway (FIG. 7K). The system responds to starvation, a classical inducer of autophagy, and engages ATG16L1-positive endosomal vesicles (Lystad et al., 2019; Moreau et al., 2011; Ravikumar et al., 2010; Travassos et al., 2010), which fuse with FIP200-positive ER/Golgi-derived membranes. Thus, mammalian cells commit to autophagy through a regulated intermixing of two membrane sources that are normally not intended to directly communicate, one working vectorially within the secretory pathway and the other running in the opposite direction via the endocytic pathway.

The regulation of the FIP200 complex by mTOR (Ganley et al., 2009; Hosokawa et al., 2009; Jung et al., 2009; Kim et al., 2011) and AMPK (Egan et al., 2011; Inoki et al., 2012, Kim et al., 2011), which extends to phosphorylation of ATG16L1 by ULK1 (Alsaadi et al., 2019), integrates metabolic inputs (Deretic and Kroemer, 2021) into HyPAS formation. FIP200 and ATG16L1 connect HyPAS to immunity and inflammation, further extended by TBK1 (Chauhan et al., 2015; Pilli et al., 2012; Ravenhill et al., 2019; Saitoh et al., 2008; Shi et al., 2020; Thurston et al., 2009; Travassos et al., 2010; Vargas et al., 2019; Wild et al., 2011). TBK1 controls STX17 (Kumar et al., 2019), a centerpiece of the HyPAS apparatus.

A long-standing question in mammalian autophagy has been where do the autophagosomal membranes come from? For the most part, two primary origins of mammalian autophagic membranes have been considered: (i) ER-centric (Axe et al., 2008; Hara et al., 2008; Hayashi-Nishino et al., 2009; Itakura and Mizushima, 2010, 2011; Mizushima et al., 2011; Nishimura et al., 2017; Tooze and Yoshimori, 2010) or (ii) PM and endosomal-centric (Knaevelsrud et al., 2013; Longatti et al., 2012; Moreau et al., 2011; Puri et al., 2013; Puri et al., 2018; Ravikumar et al., 2010; Soreng et al., 2018). Our study provides the physical and mechanistic link between the two disparate theses and explains how they can both be correct. This accommodates the reported role of ER-Golgi intermediate compartment and COPII vesicles (Ge et al., 2013; Ge et al., 2014) along with the effects of FIP200 (Ge et al., 2017) and the associated tubulovesicular morphology (Hayashi-Nishino et al., 2009). Additional contributors to autophagosomal membranes have been reported (Hailey et al., 2010; Hamasaki et al., 2013; Nascimbeni et al., 2017; Nishida et al., 2009), which may participate at the HyPAS or other stages.

HyPAS is formed via SNARE-dependent fusion centered upon STX17 and is affected by Ca²⁺. The role of Ca²⁺ along the autophagosomal-autolysosomal continuum is complex and acts as a positive or negative regulator depending on the checkpoint reached: (i) Prolonged Ca²⁺ influx from ER to mitochondria (mitochondria-associated ER-membranes, MAM) (Hayashi and Su, 2007) is necessary to maintain mitochondrial function, and when it is disrupted (Cardenas et al., 2010; Criollo et al., 2007) this induces AMPK and autophagy (Cardenas et al., 2010). (ii) As we show, Ca²⁺ is a positive co-factor for HyPAS formation with all three STX17 partners within the HyPAS apparatus, SIGMAR1 (Hayashi and Su, 2007), E-SYT2 (Saheki et al., 2016), and SERCA2 (MacLennan and Kranias, 2003; Periasamy and Kalyanasundaram, 2007; Vandecaetsbeek et al., 2011) being known regulators or effectors of Ca². SIGMAR1 redistributes in response to Ca²⁺ from the MAM to the entire ER (Hayashi and Su, 2007), thus becoming available to participate in HyPAS formation. (iii) Ca²⁺ is a negative regulator of phagophore separation from membranes to which they are initially tethered (Bissa and Deretic, 2018; Zhao et al., 2017). (iv) Pharmacological inhibition of SERCA prevents fusion between autophagosomes and lysosome (Ganley et al., 2011; Mauvezin et al., 2015). Thus, Ca²⁺ transients are necessary to move the autophagy pathway from its beginning to its completion.

STX17 role in HyPAS formation is compatible with studies suggesting that it functions in a number of ways, including autophagic initiation (Kumar et al., 2019) at mitochondria-ER contact sites (Arasaki et al., 2018; Hamasaki et al., 2013), where another HyPAS component, SIGMAR1 operates (Hayashi and Su, 2007). Prior studies (Diao et al., 2015; Guo et al., 2014; Itakura et al., 2012; Takats et al., 2013; Wang et al., 2016) and recent work (Gu et al., 2019; Kumar et al., 2020; Kumar et al., 2018; Yang et al., 2019) have associated STX17 with autolysosomal biogenesis. The function of STX17 in autophagosome-lysosome fusion as a sole Qₐ SNARE has been contested and requires a contribution of additional non-cognate SNAREs such as Ykt6 (Bas et al., 2018; Gao et al., 2018; Matsui et al., 2018b; Takats et al., 2018) and Stx16 (Gu et al., 2019), because STX17 inactivation alone does not prevent autophagic cargo degradation (Gu et al., 2019; Matsui et al., 2018b). STX17 orchestrates progression of the autophagy pathway, whereby different combinations of STX17 and its R-SNARE partners catalyze sequential stages. During HyPAS formation, STX17 favors VAMP7 over VAMP8, which acts in autolysosome formation (Diao et al., 2015; Itakura et al., 2012; Wang et al., 2016; Yang et al., 2019). However, SNARE redundancy or compensation in HyPAS formation is possible.

Autophagy and coronaviruses are intertwined (Cottam et al., 2011; Cottam et al., 2014; Fung and Liu, 2019, Guo et al., 2016; Guo et al., 2017; Ko et al., 2017, Prentice et al., 2004; Reggiori et al., 2010; Zhu et al., 2016) (Schneider et al., 2012; Zhao et al., 2007). Coronaviruses actively remodel cellular membranes and generate protrusion-type viral-replication compartments (VRCs) (Strating and van Kuppeveld, 2017). The coronavirus VRCs include interconnected double membrane vesicles (DMVs), vesicle packets of merged DMVs, additional convoluted membranes (CM), and represent the cellular locales for RNA replication-transcription complexes (RTCs) (EA and Jones, 2019; Knoops et al., 2008; Sola et al., 2015). Some aspects of coronavirus VRCs include morphological features of autophagosomes (e.g. DMVs) (Snijder et al., 2006) and engage peripheral autophagy factors TMEM41B and VMP1 (Schneider et al., 2021) but are distinct from classical autophagosomes (Cottam et al., 2014; Reggiori et al., 2010). The inhibition of HyPAS by SARS-CoV-2 nsp6 suggests potential diversion of membrane sources engaged in autophagy toward the formation of DMVs and CMs to support coronavirus RTCs.

Of the three proteins nsp3, nsp4, and nsp6, implicated in coronavirus remodeling of host membranes to generate VRCs (Fung and Liu, 2019; Reggiori et al., 2010) (Angelini et al., 2013; Snijder et al., 2020; Wolff et al., 2020), we focused on nsp6. This was based on nsp6's action alone on LC3 profiles (Cottam et al., 2014), the dearth of nsp3 and nsp4 host protein interactors relative to the rich portfolio of nsp6 partners (Stukalov, 2020), and the specialized engagement of nsp3 and nsp4 in VRCs and DMV pores (Angelini et al., 2013; Snijder et al., 2020; Wolff et al., 2020). Our own proximity biotinylation proteomic analysis (MSV000087840) with SARS-CoV-2 nsp6 indicates multiple interactions between nsp6 and components of the HyPAS fusion apparatus (E-SYT2, VAMP7, SIGMAR1) and regulators of HyPAS formation including SERCA2 (Ca²⁺ pump with Ca²⁺ being a key signal for HyPAS formation), TBK1 (a protein kinase phosphorylating and authorizing STX17 to associate with VAMP7), and a number of key regulators of autophagy, mTOR and AMPK (Table ST1B,C). Conversely, factors controlling fusion processes along the endosomal organelles where ATG16L1 is located, i.e. ORF3a, did not affect HyPAS formation, indicating that nsp6 domain of action is mostly within the early secretory pathway, albeit we noticed nsp6 interactions with a number of Golgi proteins (Table ST1C).

The definition of HyPAS in this work is of significance not only as a potential target for pharmacological intervention in COVID-19 but is of fundamental value for our understanding of the formation of autophagosomes, and hence for many physiological functions and disease states affected by autophagy.

### REFERENCES

Alsaadi, R. M., Losier, T. T., Tian, W., Jackson, A., Guo, Z., Rubinsztein, D. C., and Russell, R. C. (2019). ULK1-mediated phosphorylation of ATG16L1 promotes xenophagy, but destabilizes the ATG16L1 Crohn's mutant. EMBO Rep 20, e46885.
An, H., and Harper, J. W. (2018). Systematic analysis of ribophagy in human cells reveals bystander flux during selective autophagy. Nat Cell Biol 20, 135-143.
An, H., Ordureau, A., Korner, M., Paulo, J. A., and Harper, J. W. (2020). Systematic quantitative analysis of ribosome inventory during nutrient stress. Nature 583, 303-309.
Angelini, M. M., Akhlaghpour, M., Neuman, B. W., and Buchmeier, M. J. (2013). Severe acute respiratory syndrome coronavirus nonstructural proteins 3, 4, and 6 induce double-membrane vesicles. mBio 4.
Arasaki, K., Nagashima, H., Kurosawa, Y., Kimura, H., Nishida, N., Dohmae, N., Yamamoto, A., Yanagi, S., Wakana, Y., Inoue, H., et al. (2018). MAP1B-LC1 prevents autophagosome formation by linking syntaxin 17 to microtubules. EMBO Rep.
Axe, E. L., Walker, S. A., Manifava, M., Chandra, P., Roderick, H. L., Habermann, A., Griffiths, G., and Ktistakis, N. T. (2008). Autophagosome formation from membrane compartments enriched in phosphatidylinositol 3-phosphate and dynamically connected to the endoplasmic reticulum. J Cell Biol 182, 685-701.
Bakula, D., Muller, A. J., Zuleger, T., Takacs, Z., Franz-Wachtel, M., Thost, A. K., Brigger, D., Tschan, M. P., Frickey, T., Robenek, H., el al. (2017). WIPI3 and WIPI4 beta-propellers are scaffolds for LKB1-AMPK-TSC signalling circuits in the control of autophagy. Nat Commun 8, 15637.
Bas, L., Papinski, D., Licheva, M., Torggler, R., Rohringer, S., Schuschnig, M., and Kraft, C. (2018). Reconstitution reveals Ykt6 as the autophagosomal SNARE in autophagosome-vacuole fusion. J Cell Biol.
Baskaran, S., Carlson, L. A., Stjepanovic, G., Young, L. N., Kim do, J., Grob, P., Stanley, R. E., Nogales, E., and Hurley, J. H. (2014). Architecture and dynamics of the autophagic phosphatidylinositol 3-kinase complex. Elife 3.
Bissa, B., and Deretic, V. (2018). Autophagosome Formation: Cutting the Gordian Knot at the ER. Curr Biol 28, R347-R349.
Bjorkoy, G., Lamark, T., Brech, A., Outzen, H., Perander, M., Overvatn, A., Stenmark, H., and Johansen, T. (2005). p62/SQSTM1 forms protein aggregates degraded by autophagy and has a protective effect on huntingtin-induced cell death. J Cell Biol 171, 603-614.
Boncompain, G., Divoux, S., Gareil, N., de Forges, H., Lescure, A., Latreche, L., Mercanti, V., Jollivet, F., Raposo, G., and Perez, F. (2012). Synchronization of secretory protein traffic in populations of cells. Nat Methods 9, 493-498.
Bradfute, S. B., Hurwitz, I., Yingling, A. V., Ye, C., Cheng, Q., Noonan, T. P., Raval, J. S., Sosa, N. R., Mertz, G. J., Perkins, D. J., el al. (2020). Severe Acute Respiratory Syndrome Coronavirus 2 Neutralizing Antibody Titers in Convalescent Plasma and Recipients in New Mexico: An Open Treatment Study in Patients With Coronavirus Disease 2019. J Infect Dis 222, 1620-1628.
Brewer, K. D., Bacaj, T., Cavalli, A., Camilloni, C., Swarbrick, J. D., Liu, J., Zhou, A., Zhou, P., Barlow, N., Xu, J., el al. (2015). Dynamic binding mode of a Synaptotagmin-1-SNARE complex in solution. Nat Struct Mol Biol 22, 555-564.
Brose, N., Petrenko, A. G., Sudhof, T. C., and Jahn, R. (1992). Synaptotagmin: a calcium sensor on the synaptic vesicle surface. Science 256, 1021-1025.
Cardenas, C., Miller, R. A., Smith, I., Bui, T., Molgo, J., Muller, M., Vais, H., Cheung, K. H., Yang, J., Parker, I., et al. (2010). Essential regulation of cell bioenergetics by constitutive InsP3 receptor Ca2+ transfer to mitochondria. Cell 142, 270-283.
Cardona, A., Saalfeld, S., Schindelin, J., Arganda-Carreras, I., Preibisch, S., Longair, M., Tomancak, P., Hartenstein, V., and Douglas, R. J. (2012). TrakEM2 software for neural circuit reconstruction. PLoS One 7, e38011.
Chang, C., Young, L. N., Morris, K. L., von Bulow, S., Schoneberg, J., Yamamoto-Imoto, H., Oe, Y., Yamamoto, K., Nakamura, S., Stjepanovic, G., et al. (2019). Bidirectional Control of Autophagy by BECN1 BARA Domain Dynamics. Mol Cell 73, 339-353 e336.
Chapman, E. R. (2002). Synaptotagmin: a Ca(2+) sensor that triggers exocytosis? Nat Rev Mol Cell Biol 3, 498-508.
Chauhan, S., Kumar, S., Jain, A., Ponpuak, M., Mudd, M. H., Kimura, T., Choi, S. W., Peters, R., Mandell, M., Bruun, J. A., et al. (2016). TRIMs and Galectins Globally Cooperate and TRIM16 and Galectin-3 Co-direct Autophagy in Endomembrane Damage Homeostasis. Dev Cell 39, 13-27.
Chauhan, S., Mandell, M. A., and Deretic, V. (2015). IRGM Governs the Core Autophagy Machinery to Conduct Antimicrobial Defense. Mol Cell 58, 507-521.
Christ, M. G., Huesmann, H., Nagel, H., Kern, A., and Behl, C. (2019). Sigma-1 Receptor Activation Induces Autophagy and Increases Proteostasis Capacity In Vitro and In Vivo. Cells 8.
Cottam, E. M., Maier, H. J., Manifava, M., Vaux, L. C., Chandra-Schoenfelder, P., Gerner, W., Britton, P., Ktistakis, N. T., and Wileman, T. (2011). Coronavirus nsp6 proteins generate autophagosomes from the endoplasmic reticulum via an omegasome intermediate. Autophagy 7, 1335-1347.
Cottam, E. M., Whelband, M. C., and Wileman, T. (2014). Coronavirus NSP6 restricts autophagosome expansion. Autophagy 10, 1426-1441.
Criollo, A., Maiuri, M. C., Tasdemir, E., Vitale, I., Fiebig, A. A., Andrews, D., Molgo, J., Diaz, J., Lavandero, S., Harper, F., et al. (2007). Regulation of autophagy by the inositol trisphosphate receptor. Cell Death Differ 14, 1029-1039.
Deretic, V. (2021). Autophagy in inflammation, infection, and immunometabolism. Immunity 54, 437-453.
Deretic, V., and Kroemer, G. (2021). Autophagy in metabolism and quality control: opposing, complementary or interlinked functions? Autophagy, 1-10.
Diao, J., Liu, R., Rong, Y., Zhao, M., Zhang, J., Lai, Y., Zhou, Q., Wilz, L. M., Li, J., Vivona, S., et al. (2015). ATG14 promotes membrane tethering and fusion of autophagosomes to endolysosomes. Nature 520, 563-566.
Dooley, H. C., Razi, M., Polson, H. E., Girardin, S. E., Wilson, M. I., and Tooze, S. A. (2014). WIPI2 Links LC3 Conjugation with PI3P, Autophagosome Formation, and Pathogen Clearance by Recruiting Atg12-5-16L1. Mol Cell 55, 238-252.
EA, J. A., and Jones, I. M. (2019). Membrane binding proteins of coronaviruses. Future Virol 14, 275-286.
Egan, D. F., Shackelford, D. B., Mihaylova, M. M., Gelino, S., Kohnz, R. A., Mair, W., Vasquez, D. S., Joshi, A., Gwinn, D. M., Taylor, R., et al. (2011). Phosphorylation of ULK1 (hATG1) by AMP-activated protein kinase connects energy sensing to mitophagy. Science 331, 456-461.
Engedal, N., Torgersen, M. L., Guldvik, I. J., Barfeld, S. J., Bakula, D., Saetre, F., Hagen, L. K., Patterson, J. B., Proikas-Cezanne, T., Seglen, P. O., et al. (2013). Modulation of intracellular calcium homeostasis blocks autophagosome formation. Autophagy 9, 1475-1490.
Eskelinen, E. L. (2008). Fine structure of the autophagosome. Methods Mol Biol 445, 11-28.
Feher, A., Juhasz, A., Laszlo, A., Kalman, J., Jr., Pakaski, M., Kalman, J., and Janka, Z. (2012). Association between a variant of the sigma-1 receptor gene and Alzheimer's disease. Neurosci Lett 517, 136-139.
Fletcher, K., Ulferts, R., Jacquin, E., Veith, T., Gammoh, N., Arasteh, J. M., Mayer, U., Carding, S. R., Wileman, T., Beale, R., et al. (2018). The WD40 domain of ATG16L1 is required for its non-canonical role in lipidation of LC3 at single membranes. EMBO J 37.
Fujita, N., Morita, E., Itoh, T., Tanaka, A., Nakaoka, M., Osada, Y., Umemoto, T., Saitoh, T., Nakatogawa, H., Kobayashi, S., et al. (2013). Recruitment of the autophagic machinery to endosomes during infection is mediated by ubiquitin. J Cell Biol 203, 115-128.
Fung, T. S., and Liu, D. X. (2019). Human Coronavirus: Host-Pathogen Interaction. Annu Rev Microbiol 73, 529-557.
Galluzzi, L., and Green, D. R. (2019). Autophagy-Independent Functions of the Autophagy Machinery. Cell 177, 1682-1699.
Gammoh, N., Florey, O., Overholtzer, M., and Jiang, X. (2013). Interaction between FIP200 and ATG16L1 distinguishes ULK1 complex-dependent and -independent autophagy. Nat Struct Mol Biol 20, 144-149.
Ganley, I. G., Lam du, H., Wang, J., Ding, X., Chen, S., and Jiang, X. (2009). ULK1.ATG13.FIP200 complex mediates mTOR signaling and is essential for autophagy. J Biol Chem 284, 12297-12305.
Ganley, I. G., Wong, P. M., Gammoh, N., and Jiang, X. (2011). Distinct autophagosomal-lysosomal fusion mechanism revealed by thapsigargin-induced autophagy arrest. Mol Cell 42, 731-743.
Gao, J., Reggiori, F., and Ungermann, C. (2018). A novel in vitro assay reveals SNARE topology and the role of Ykt6 in autophagosome fusion with vacuoles. J Cell Biol.
Gassen, N. C., Niemeyer, D., Muth, D., Corman, V. M., Martinelli, S., Gassen, A., Hafner, K., Papies, J., Mosbauer, K., Zellner, A., et al. (2019). SKP2 attenuates autophagy through Beclin 1-ubiquitination and its inhibition reduces MERS-Coronavirus infection. Nature communications 10, 5770.
Ge, L., Melville, D., Zhang, M., and Schekman, R. (2013). The ER-Golgi intermediate compartment is a key membrane source for the LC3 lipidation step of autophagosome biogenesis. Elife 2, e00947.
Ge, L., Zhang, M., Kenny, S. J., Liu, D., Maeda, M., Saito, K., Mathur, A., Xu, K., and Schekman, R. (2017). Remodeling of ER-exit sites initiates a membrane supply pathway for autophagosome biogenesis. EMBO Rep 18, 1586-1603.
Ge, L., Zhang, M., and Schekman, R. (2014). Phosphatidylinositol 3-kinase and COPII generate LC3 lipidation vesicles from the ER-Golgi intermediate compartment. Elife 3, e04135.
Ghosh, S., Dellibovi-Ragheb, T. A., Kerviel, A., Pak, E., Qiu, Q., Fisher, M., Takvorian, P. M., Bleck, C., Hsu, V. W., Fehr, A. R., et al. (2020). beta-Coronaviruses Use Lysosomes for Egress Instead of the Biosynthetic Secretory Pathway. Cell.
Giordano, F., Saheki, Y., Idevall-Hagren, O., Colombo, S. F., Pirruccello, M., Milosevic, I., Gracheva, E. O., Bagriantsev, S. N., Borgese, N., and De Camilli, P. (2013). PI(4,5)P(2)-dependent and Ca(2+)-regulated ER-PM interactions mediated by the extended synaptotagmins. Cell 153, 1494-1509.
Gordon, D. E., Jang, G. M., Bouhaddou, M., Xu, J., Obernier, K., White, K. M., O'Meara, M. J., Rezelj, V. V., Guo, J. Z., Swaney, D. L., et al. (2020). A SARS-CoV-2 protein interaction map reveals targets for drug repurposing. Nature.
Gu, Y., Princely Abudu, Y., Kumar, S., Bissa, B., Choi, S. W., Jia, J., Lazarou, M., Eskelinen, E. L., Johansen, T., and Deretic, V. (2019). Mammalian Atg8 proteins regulate lysosome and autolysosome biogenesis through SNAREs. EMBO J 38, e101994.
Guo, B., Liang, Q., Li, L., Hu, Z., Wu, F., Zhang, P., Ma, Y., Zhao, B., Kovacs, A. L., Zhang, Z., et al. (2014). O-GlcNAc-modification of SNAP-29 regulates autophagosome maturation. Nat Cell Biol 16, 1215-1226.
Guo, L., Yu, H., Gu, W., Luo, X., Li, R., Zhang, J., Xu, Y., Yang, L., Shen, N., Feng, L., et al. (2016). Autophagy Negatively Regulates Transmissible Gastroenteritis Virus Replication. Sci Rep 6, 23864.
Guo, X., Zhang, M., Zhang, X., Tan, X., Guo, H., Zeng, W., Yan, G., Memon, A. M., Li, Z., Zhu, Y., et al. (2017). Porcine Epidemic Diarrhea Virus Induces Autophagy to Benefit Its Replication. Viruses 9.
Hailey, D. W., Rambold, A. S., Satpute-Krishnan, P., Mitra, K., Sougrat, R., Kim, P. K., and Lippincott-Schwartz, J. (2010). Mitochondria supply membranes for autophagosome biogenesis during starvation. Cell 141, 656-667.
Hamasaki, M., Furuta, N., Matsuda, A., Nezu, A., Yamamoto, A., Fujita, N., Oomori, H., Noda, T., Haraguchi, T., Hiraoka, Y., et al. (2013). Autophagosomes form at ER-mitochondria contact sites. Nature 495, 389-393.
Hara, T., Takamura, A., Kishi, C., Iemura, S., Natsume, T., Guan, J. L., and Mizushima, N. (2008). FIP200, a ULK-interacting protein, is required for autophagosome formation in mammalian cells. J Cell Biol 181, 497-510.
Hayashi, T., and Su, T. P. (2007). Sigma-1 receptor chaperones at the ER-mitochondrion interface regulate Ca(2+) signaling and cell survival. Cell 131, 596-610.
Hayashi-Nishino, M., Fujita, N., Noda, T., Yamaguchi, A., Yoshimori, T., and Yamamoto, A. (2009). A subdomain of the endoplasmic reticulum forms a cradle for autophagosome formation. Nat Cell Biol 11, 1433-1437.
Heckmann, B. L., Teubner, B. J. W., Tummers, B., Boada-Romero, E., Harris, L., Yang, M., Guy, C. S., Zakharenko, S. S., and Green, D. R. (2019). LC3-Associated Endocytosis Facilitates beta-Amyloid Clearance and Mitigates Neurodegeneration in Murine Alzheimer's Disease. Cell 178, 536-551 e514.
Hirata, Y., Yamamoto, H., Atta, M. S., Mahmoud, S., Oh-hashi, K., and Kiuchi, K. (2011). Chloroquine inhibits glutamate-induced death of a neuronal cell line by reducing reactive oxygen species through sigma-1 receptor. J Neurochem 119, 839-847.
Hoffmann, H. H., Schneider, W. M., Rozen-Gagnon, K., Miles, L. A., Schuster, F., Razooky, B., Jacobson, E., Wu, X., Yi, S., Rudin, C. M., et al. (2021). TMEM41B Is a Pan-flavivirus Host Factor. Cell 184, 133-148 e120.
Hoffmann, M., Kleine-Weber, H., Schroeder, S., Kruger, N., Herrler, T., Erichsen, S., Schiergens, T. S., Herrier, G., Wu, N. H., Nitsche, A., et al. (2020a). SARS-CoV-2 Cell Entry Depends on ACE2 and TMPRSS2 and Is Blocked by a Clinically Proven Protease Inhibitor. Cell 181, 271-280 e278.
Hoffmann, M., Mosbauer, K., Hofmann-Winkler, H., Kaul, A., Kleine-Weber, H., Kruger, N., Gassen, N. C., Muller, M. A., Drosten, C., and Pohlmann, S. (2020b). Chloroquine does not inhibit infection of human lung cells with SARS-CoV-2. Nature.
Hosokawa, N., Hara, T., Kaizuka, T., Kishi, C., Takamura, A., Miura, Y., Iemura, S., Natsume, T., Takehana, K., Yamada, N., et al. (2009). Nutrient-dependent mTORC1 association with the ULK1-Atg13-FIP200 complex required for autophagy. Mol Biol Cell 20, 1981-1991.
Inoki, K., Kim, J., and Guan, K. L. (2012). AMPK and mTOR in cellular energy homeostasis and drug targets. Annu Rev Pharmacol Toxicol 52, 381-400.
Itakura, E., Kishi-Itakura, C., and Mizushima, N. (2012). The hairpin-type tail-anchored SNARE syntaxin 17 targets to autophagosomes for fusion with endosomes/lysosomes. Cell 151, 1256-1269.
Itakura, E., and Mizushima, N. (2010). Characterization of autophagosome formation site by a hierarchical analysis of mammalian Atg proteins. Autophagy 6.
Itakura, E., and Mizushima, N. (2011). p62 Targeting to the autophagosome formation site requires self-oligomerization but not LC3 binding. The Journal of cell biology 192, 17-27.
Jacquin, E., Leclerc-Mercier, S., Judon, C., Blanchard, E., Fraitag, S., and Florey, O. (2017). Pharmacological modulators of autophagy activate a parallel noncanonical pathway driving unconventional LC3 lipidation. Autophagy 13, 854-867.
Jung, C. H., Jun, C. B., Ro, S. H., Kim, Y. M., Otto, N. M., Cao, J., Kundu, M., and Kim, D. H. (2009). ULK-Atg13-FIP200 complexes mediate mTOR signaling to the autophagy machinery. Mol Biol Cell 20, 1992-2003.
Katayama, H., Kogure, T., Mizushima, N., Yoshimori, T., and Miyawaki, A. (2011). A sensitive and quantitative technique for detecting autophagic events based on lysosomal delivery. Chem Biol 18, 1042-1052.
Kim, J., Kundu, M., Viollet, B., and Guan, K. L. (2011). AMPK and mTOR regulate autophagy through direct phosphorylation of Ulk1. Nat Cell Biol 13, 132-141.
Klionsky, D. J., Abdelmohsen, K., Abe, A., Abedin, M. J., Abeliovich, H., Acevedo Arozena, A., Adachi, H., Adams, C. M., Adams, P. D., Adeli, K., et al. (2016). Guidelines for the use and interpretation of assays for monitoring autophagy (3rd edition). Autophagy 12, 1-222.
Klionsky, D. J., Petroni, G., Amaravadi, R. K., Baehrecke, E. H., Ballabio, A., Boya, P., Bravo-San Pedro, J. M., Cadwell, K., Cecconi, F., Choi, A. M. K., et al. (2021). Autophagy in major human diseases. EMBO J, e108863.
Knaevelsrud, H., Soreng, K., Raiborg, C., Haberg, K., Rasmuson, F., Brech, A., Liestol, K., Rusten, T. E., Stenmark, H., Neufeld, T. P., et al. (2013). Membrane remodeling by the PX-BAR protein SNX18 promotes autophagosome formation. J Cell Biol 202, 331-349.
Knoops, K., Kikkert, M., Worm, S. H., Zevenhoven-Dobbe, J. C., van der Meer, Y., Koster, A. J., Mommaas, A. M., and Snijder, E. J. (2008). SARS-coronavirus replication is supported by a reticulovesicular network of modified endoplasmic reticulum. PLoS Biol 6, e226.
Ko, S., Gu, M. J., Kim, C. G., Kye, Y. C., Lim, Y., Lee, J. E., Park, B. C., Chu, H., Han, S. H., and Yun, C. H. (2017). Rapamycin-induced autophagy restricts porcine epidemic diarrhea virus infectivity in porcine intestinal epithelial cells. Antiviral Res 146, 86-95.
Kopitz, J., Kisen, G. O., Gordon, P. B., Bohley, P., and Seglen, P. O. (1990). Nonselective autophagy of cytosolic enzymes by isolated rat hepatocytes. J Cell Biol 111, 941-953.
Kumar, S., Gu, Y., Abudu, Y. P., Bruun, J. A., Jain, A., Farzam, F., Mudd, M., Anonsen, J. H., Rusten, T. E., Kasof, G., et al. (2019). Phosphorylation of Syntaxin 17 by TBK1 Controls Autophagy Initiation. Dev Cell.
Kumar, S., Jain, A., Choi, S. W., da Silva, G. P. D., Allers, L., Mudd, M. H., Peters, R. S., Anonsen, J. H., Rusten, T. E., Lazarou, M., et al. (2020). Mammalian Atg8 proteins and the autophagy factor IRGM control mTOR and TFEB at a regulatory node critical for responses to pathogens. Nat Cell Biol 22, 973-985.
Kumar, S., Jain, A., Farzam, F., Jia, J., Gu, Y., Choi, S. W., Mudd, M. H., Claude-Taupin, A., Wester, M. J., Lidke, K. A., et al. (2018). Mechanism of Stx17 recruitment to autophagosomes via IRGM and mammalian Atg8 proteins. J Cell Biol 217, 997-1013.
Lamb, C. A., Yoshimori, T., and Tooze, S. A. (2013). The autophagosome: origins unknown, biogenesis complex. Nat Rev Mol Cell Biol 14, 759-774.
Lazarou, M., Sliter, D. A., Kane, L. A., Sarraf, S. A., Wang, C., Burman, J. L., Sideris, D. P., Fogel, A. I., and Youle, R. J. (2015). The ubiquitin kinase PINK1 recruits autophagy receptors to induce mitophagy. Nature 524, 309-314.
Levine, B., and Kroemer, G. (2019). Biological Functions of Autophagy Genes: A Disease Perspective. Cell 176, 11-42.
Longatti, A., Lamb, C. A., Razi, M., Yoshimura, S., Barr, F. A., and Tooze, S. A. (2012). TBC1D14 regulates autophagosome formation via Rab11- and ULK1-positive recycling endosomes. J Cell Biol 197, 659-675.
Lystad, A. H., Carlsson, S. R., de la Ballina, L. R., Kauffman, K. J., Nag, S., Yoshimori, T., Melia, T. J., and Simonsen, A. (2019). Distinct functions of ATG16L1 isoforms in membrane binding and LC3B lipidation in autophagy-related processes. Nat Cell Biol 21, 372-383.
MacLennan, D. H., and Kranias, E. G. (2003). Phospholamban: a crucial regulator of cardiac contractility. Nat Rev Mol Cell Biol 4, 566-577.
Maher, C. M., Thomas, J. D., Haas, D. A., Longen, C. G., Oyer, H. M., Tong, J. Y., and Kim, F. J. (2018). Small-Molecule Sigma1 Modulator Induces Autophagic Degradation of PD-L1. Mol Cancer Res 16, 243-255.
Martinez, J., Malireddi, R. K., Lu, Q., Cunha, L. D., Pelletier, S., Gingras, S., Orchard, R., Guan, J. L., Tan, H., Peng, J., el al. (2015). Molecular characterization of LC3-associated phagocytosis reveals distinct roles for Rubicon, NOX2 and autophagy proteins. Nat Cell Biol 17, 893-906.
Matsui, T., Jiang, P., Nakano, S., Sakamaki, Y., Yamamoto, H., and Mizushima, N. (2018a). Autophagosomal YKT6 is required for fusion with lysosomes independently of syntaxin 17. J Cell Biol.
Matsui, T., Jiang, P., Nakano, S., Sakamaki, Y., Yamamoto, H., and Mizushima, N. (2018b). Autophagosomal YKT6 is required for fusion with lysosomes independently of syntaxin 17. J Cell Biol 217, 2633-2645.
Mauthe, M., Orhon, I., Rocchi, C., Zhou, X., Luhr, M., Hijlkema, K. J., Coppes, R. P., Engedal, N., Mari, M., and Reggiori, F. (2018). Chloroquine inhibits autophagic flux by decreasing autophagosome-lysosome fusion. Autophagy 14, 1435-1455.
Mauvezin, C., Nagy, P., Juhasz, G., and Neufeld, T. P. (2015). Autophagosome-lysosome fusion is independent of V-ATPase-mediated acidification. Nat Commun 6, 7007.
Melia, T. J., Lystad, A. H., and Simonsen, A. (2020). Autophagosome biogenesis: From membrane growth to closure. J Cell Biol 219.
Miao, G., Zhao, H., Li, Y., Ji, M., Chen, Y., Shi, Y., Bi, Y., Wang, P., and Zhang, H. (2021). ORF3a of the COVID-19 virus SARS-CoV-2 blocks HOPS complex-mediated assembly of the SNARE complex required for autolysosome formation. Dev Cell 56, 427-442 e425.
Min, S. W., Chang, W. P., and Sudhof, T. C. (2007). E-Syts, a family of membranous Ca2+-sensor proteins with multiple C2 domains. Proc Natl Acad Sci USA 104, 3823-3828.
Mizushima, N., Yoshimori, T., and Ohsumi, Y. (2011). The role of Atg proteins in autophagosome formation. Annu Rev Cell Dev Biol 27, 107-132.
Moreau, K., Ravikumar, B., Renna, M., Puri, C., and Rubinsztein, D. C. (2011). Autophagosome precursor maturation requires homotypic fusion. Cell 146, 303-317.
Morishita, H., and Mizushima, N. (2019). Diverse Cellular Roles of Autophagy. Annu Rev Cell Dev Biol 35, 453-475.
Narendra, D., Tanaka, A., Suen, D. F., and Youle, R. J. (2008). Parkin is recruited selectively to impaired mitochondria and promotes their autophagy. J Cell Biol 183, 795-803.
Nascimbeni, A. C., Giordano, F., Dupont, N., Grasso, D., Vaccaro, M. I., Codogno, P., and Morel, E. (2017). ER-plasma membrane contact sites contribute to autophagosome biogenesis by regulation of local PI3P synthesis. EMBO J 36, 2018-2033.
Nguyen, T. N., Padman, B. S., Usher, J., Oorschot, V., Ramm, G., and Lazarou, M. (2016). Atg8 family LC3/GABARAP proteins are crucial for autophagosome-lysosome fusion but not autophagosome formation during PINK1/Parkin mitophagy and starvation. J Cell Biol 215, 857-874.
Nishida, Y., Arakawa, S., Fujitani, K., Yamaguchi, H., Mizuta, T., Kanaseki, T., Komatsu, M., Otsu, K., Tsujimoto, Y., and Shimizu, S. (2009). Discovery of Atg5/Atg7-independent alternative macroautophagy. Nature 461, 654-658.
Nishimura, T., Kaizuka, T., Cadwell, K., Sahani, M. H., Saitoh, T., Akira, S., Virgin, H. W., and Mizushima, N. (2013). FIP200 regulates targeting of Atg16L1 to the isolation membrane. EMBO Rep 14, 284-291.
Nishimura, T., Tamura, N., Kono, N., Shimanaka, Y., Arai, H., Yamamoto, H., and Mizushima, N. (2017). Autophagosome formation is initiated at phosphatidylinositol synthase-enriched ER subdomains. EMBO J 36, 1719-1735.
Pankiv, S., Clausen, T. H., Lamark, T., Brech, A., Bruun, J. A., Outzen, H., Overvatn, A., Bjorkoy, G., and Johansen, T. (2007). p62/SQSTM1 binds directly to Atg8/LC3 to facilitate degradation of ubiquitinated protein aggregates by autophagy. J Biol Chem 282, 24131-24145.
Pattingre, S., Bauvy, C., and Codogno, P. (2003). Amino acids interfere with the ERK1/2-dependent control of macroautophagy by controlling the activation of Raf-1 in human colon cancer HT-29 cells. J Biol Chem 278, 16667-16674.
Periasamy, M., and Kalyanasundaram, A. (2007). SERCA pump isoforms: their role in calcium transport and disease. Muscle Nerve 35, 430-442.
Pilli, M., Arko-Mensah, J., Ponpuak, M., Roberts, E., Master, S., Mandell, M. A., Dupont, N., Ornatowski, W., Jiang, S., Bradfute, S. B., et al. (2012). TBK-1 Promotes Autophagy-Mediated Antimicrobial Defense by Controlling Autophagosome Maturation. Immunity 37, 223-234.
Prentice, E., Jerome, W. G., Yoshimori, T., Mizushima, N., and Denison, M. R. (2004). Coronavirus replication complex formation utilizes components of cellular autophagy. J Biol Chem 279, 10136-10141.
Puri, C., Renna, M., Bento, C. F., Moreau, K., and Rubinsztein, D. C. (2013). Diverse autophagosome membrane sources coalesce in recycling endosomes. Cell 154, 1285-1299.
Puri, C., Vicinanza, M., Ashkenazi, A., Gratian, M. J., Zhang, Q., Bento, C. F., Renna, M., Menzies, F. M., and Rubinsztein, D. C. (2018). The RAB11A-Positive Compartment Is a Primary Platform for Autophagosome Assembly Mediated by WIPI2 Recognition of PI3P-RAB11A. Dev Cell 45, 114-131 e118.
Ravenhill, B. J., Boyle, K. B., von Muhlinen, N., Ellison, C. J., Masson, G. R., Otten, E. G., Foeglein, A., Williams, R., and Randow, F. (2019). The Cargo Receptor NDP52 Initiates Selective Autophagy by Recruiting the ULK Complex to Cytosol-Invading Bacteria. Mol Cell 74, 320-329 e326.
Ravikumar, B., Moreau, K., Jahreiss, L., Puri, C., and Rubinsztein, D. C. (2010). Plasma membrane contributes to the formation of pre-autophagosomal structures. Nat Cell Biol 12, 747-757.
Reggiori, F., Monastyrska, I., Verheije, M. H., Cali, T., Ulasli, M., Bianchi, S., Bernasconi, R., de Haan, C. A., and Molinari, M. (2010). Coronaviruses Hijack the LC3-I-positive EDEMosomes, ER-derived vesicles exporting short-lived ERAD regulators, for replication. Cell Host Microbe 7, 500-508.
Reggiori, F., and Ungermann, C. (2017). Autophagosome Maturation and Fusion. J Mol Biol 429, 486-496.
Saheki, Y., Bian, X., Schauder, C. M., Sawaki, Y., Surma, M. A., Klose, C., Pincet, F., Reinisch, K. M., and De Camilli, P. (2016). Control of plasma membrane lipid homeostasis by the extended synaptotagmins. Nat Cell Biol 18, 504-515.
Saitoh, T., Fujita, N., Jang, M. H., Uematsu, S., Yang, B. G., Satoh, T., Omori, H., Noda, T., Yamamoto, N., Komatsu, M., et al. (2008). Loss of the autophagy protein Atg16L1 enhances endotoxin-induced IL-1beta production. Nature 456, 264-268.
Sanjuan, M. A., Dillon, C. P., Tait, S. W., Moshiach, S., Dorsey, F., Connell, S., Komatsu, M., Tanaka, K., Cleveland, J. L., Withoff, S., et al. (2007). Toll-like receptor signalling in macrophages links the autophagy pathway to phagocytosis. Nature 450, 1253-1257.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682.
Schmidt, H. R., Zheng, S., Gurpinar, E., Koehl, A., Manglik, A., and Kruse, A. C. (2016). Crystal structure of the human sigma1 receptor. Nature 532, 527-530.
Schneider, M., Ackermann, K., Stuart, M., Wex, C., Protzer, U., Schatzl, H. M., and Gilch, S. (2012). Severe acute respiratory syndrome coronavirus replication is severely impaired by MG132 due to proteasome-independent inhibition of M-calpain. J Virol 86, 10112-10122.
Schneider, W. M., Luna, J. M., Hoffmann, H. H., Sanchez-Rivera, F. J., Leal, A. A., Ashbrook, A. W., Le Pen, J., Ricardo-Lax, I., Michailidis, E., Peace, A., et al. (2021). Genome-Scale Identification of SARS-CoV-2 and Pan-coronavirus Host Factor Networks. Cell 184, 120-132 e114.
Sclip, A., Bacaj, T., Giam, L. R., and Sudhof, T. C. (2016). Extended Synaptotagmin (ESyt) Triple Knock-Out Mice Are Viable and Fertile without Obvious Endoplasmic Reticulum Dysfunction. PLoS One 11, e0158295.
Shi, X., Yokom, A. L., Wang, C., Young, L. N., Youle, R. J., and Hurley, J. H. (2020). ULK complex organization in autophagy by a C-shaped FIP200 N-terminal domain dimer. J Cell Biol 219.
Snijder, E. J., Limpens, R., de Wilde, A. H., de Jong, A. W. M., Zevenhoven-Dobbe, J. C., Maier, H. J., Faas, F., Koster, A. J., and Barcena, M. (2020). A unifying structural and functional model of the coronavirus replication organelle: Tracking down RNA synthesis. PLoS Biol 18, e3000715.
Snijder, E. J., van der Meer, Y., Zevenhoven-Dobbe, J., Onderwater, J. J., van der Meulen, J., Koerten, H. K., and Mommaas, A. M. (2006). Ultrastructure and origin of membrane vesicles associated with the severe acute respiratory syndrome coronavirus replication complex. J Virol 80, 5927-5940.
Sola, I., Almazan, F., Zuniga, S., and Enjuanes, L. (2015). Continuous and Discontinuous RNA Synthesis in Coronaviruses. Annu Rev Virol 2, 265-288.
Soreng, K., Munson, M. J., Lamb, C. A., Bjorndal, G. T., Pankiv, S., Carlsson, S. R., Tooze, S. A., and Simonsen, A. (2018). SNX18 regulates ATG9A trafficking from recycling endosomes by recruiting Dynamin-2. EMBO Rep.
Strating, J. R., and van Kuppeveld, F. J. (2017). Viral rewiring of cellular lipid metabolism to create membranous replication compartments. Curr Opin Cell Biol 47, 24-33.
Stukalov, A., V. Girault, V. Grass, V. Bergant, O. Karayel, C. Urban, D. A. Haas, Y. Huang, L. Oubraham, A. Wang, et al. (2020). Wang, et al. 2020. Multi-level proteomics reveals host-perturbation strategies of SARS-CoV-2 and SARS-CoV. bioRxiv.
Sugo, M., Kimura, H., Arasaki, K., Amemiya, T., Hirota, N., Dohmae, N., Imai, Y., Inoshita, T., Shiba-Fukushima, K., Hattori, N., et al. (2018). Syntaxin 17 regulates the localization and function of PGAM5 in mitochondrial division and mitophagy. EMBO J.
Szalai, P., Hagen, L. K., Saetre, F., Luhr, M., Sponheim, M., Overbye, A., Mills, I. G., Seglen, P. O., and Engedal, N. (2015). Autophagic bulk sequestration of cytosolic cargo is independent of LC3, but requires GABARAPs. Exp Cell Res 333, 21-38.
Takahashi, Y., He, H., Tang, Z., Hattori, T., Liu, Y., Young, M. M., Serfass, J. M., Chen, L., Gebru, M., Chen, C., et al. (2018). An autophagy assay reveals the ESCRT-III component CHMP2A as a regulator of phagophore closure. Nature communications 9, 2855.
Takats, S., Glatz, G., Szenci, G., Boda, A., Horvath, G. V., Hegedus, K., Kovacs, A. L., and Juhasz, G. (2018). Non-canonical role of the SNARE protein Ykt6 in autophagosome-lysosome fusion. PLoS Genet 14, e1007359.
Takats, S., Nagy, P., Varga, A., Pires, K., Karpati, M., Varga, K., Kovacs, A. L., Hegedus, K., and Juhasz, G. (2013). Autophagosomal Syntaxin17-dependent lysosomal degradation maintains neuronal function in Drosophila. J Cell Biol 201, 531-539.
Tanaka, Y., Guhde, G., Suter, A., Eskelinen, E. L., Hartmann, D., Lullmann-Rauch, R., Janssen, P. M., Blanz, J., von Figura, K., and Saftig, P. (2000). Accumulation of autophagic vacuoles and cardiomyopathy in LAMP-2-deficient mice. Nature 406, 902-906.
Teis, D., Saksena, S., and Emr, S. D. (2008). Ordered assembly of the ESCRT-III complex on endosomes is required to sequester cargo during MVB formation. Dev Cell 15, 578-589.
Tesei, A., Cortesi, M., Zamagni, A., Arienti, C., Pignatta, S., Zanoni, M., Paolillo, M., Curti, D., Rui, M., Rossi, D., et al. (2018). Sigma Receptors as Endoplasmic Reticulum Stress “Gatekeepers” and their Modulators as Emerging New Weapons in the Fight Against Cancer. Front Pharmacol 9, 711.
Thurston, T. L., Ryzhakov, G., Bloor, S., von Muhlinen, N., and Randow, F. (2009). The TBK1 adaptor and autophagy receptor NDP52 restricts the proliferation of ubiquitin-coated bacteria. Nat Immunol 10, 1215-1221.
Tooze, S. A., and Yoshimori, T. (2010). The origin of the autophagosomal membrane. Nat Cell Biol 12, 831-835.
Travassos, L. H., Carneiro, L. A., Ramjeet, M., Hussey, S., Kim, Y. G., Magalhaes, J. G., Yuan, L., Soares, F., Chea, E., Le Bourhis, L., et al. (2010). Nod1 and Nod2 direct autophagy by recruiting ATG16L1 to the plasma membrane at the site of bacterial entry. Nature immunology 11, 55-62.
Vandecaetsbeek, I., Vangheluwe, P., Raeymaekers, L., Wuytack, F., and Vanoevelen, J. (2011). The Ca2+ pumps of the endoplasmic reticulum and Golgi apparatus. Cold Spring Harb Perspect Biol 3.
Vargas, J. N. S., Wang, C., Bunker, E., Hao, L., Maric, D., Schiavo, G., Randow, F., and Youle, R. J. (2019). Spatiotemporal Control of ULK1 Activation by NDP52 and TBK1 during Selective Autophagy. Mol Cell 74, 347-362 e346.
Velikkakath, A. K., Nishimura, T., Oita, E., Ishihara, N., and Mizushima, N. (2012). Mammalian Atg2 proteins are essential for autophagosome formation and important for regulation of size and distribution of lipid droplets. Molecular biology of the cell 23, 896-909.
Vilner, B. J., John, C. S., and Bowen, W. D. (1995). Sigma-1 and sigma-2 receptors are expressed in a wide variety of human and rodent tumor cell lines. Cancer Res 55, 408-413.
Violot, S., Carpentier, P., Blanchoin, L., and Bourgeois, D. (2009). Reverse pH-dependence of chromophore protonation explains the large Stokes shift of the red fluorescent protein mKeima. J Am Chem Soc 131, 10356-10357.
Vollrath, J. T., Sechi, A., Dreser, A., Katona, I., Wiemuth, D., Vervoorts, J., Dohmen, M., Chandrasekar, A., Prause, J., Brauers, E., el al. (2014). Loss of function of the ALS protein SigR1 leads to ER pathology associated with defective autophagy and lipid raft disturbances. Cell Death Dis 5, e1290.
Wang, L., Kim, J. Y., Liu, H. M., Lai, M. M. C., and Ou, J. J. (2017). HCV-induced autophagosomes are generated via homotypic fusion of phagophores that mediate HCV RNA replication. PLoS Pathog 13, e1006609.
Wang, Z., Miao, G., Xue, X., Guo, X., Yuan, C., Wang, Z., Zhang, G., Chen, Y., Feng, D., Hu, J., el al. (2016). The Vici Syndrome Protein EPG5 Is a Rab7 Effector that Determines the Fusion Specificity of Autophagosomes with Late Endosomes/Lysosomes. Mol Cell 63, 781-795.
Watanabe, S., Ilieva, H., Tamada, H., Nomura, H., Komine, O., Endo, F., Jin, S., Mancias, P., Kiyama, H., and Yamanaka, K. (2016). Mitochondria-associated membrane collapse is a common pathomechanism in SIGMAR1- and SOD1-linked ALS. EMBO Mol Med 8, 1421-1437.
Wild, P., Farhan, H., McEwan, D. G., Wagner, S., Rogov, V. V., Brady, N. R., Richter, B., Korac, J., Waidmann, O., Choudhary, C., el al. (2011). Phosphorylation of the autophagy receptor optineurin restricts Salmonella growth. Science 333, 228-233.
Wolff, G., Limpens, R., Zevenhoven-Dobbe, J. C., Laugks, U., Zheng, S., de Jong, A. W. M., Koning, R. I., Agard, D. A., Grunewald, K., Koster, A. J., et al. (2020). A molecular pore spans the double membrane of the coronavirus replication organelle. Science 369, 1395-1398.
Wyant, G. A., Abu-Remaileh, M., Frenkel, E. M., Laqtom, N. N., Dharamdasani, V., Lewis, C. A., Chan, S. H., Heinze, I., Ori, A., and Sabatini, D. M. (2018). NUFIP1 is a ribosome receptor for starvation-induced ribophagy. Science 360, 751-758.
Yang, H., Shen, H., Li, J., and Guo, L. W. (2019). SIGMAR1/Sigma-1 receptor ablation impairs autophagosome clearance. Autophagy 15, 1539-1557.
Youle, R. J. (2019). Mitochondria-Striking a balance between host and endosymbiont. Science 365.
Zechner, R., Madeo, F., and Kratky, D. (2017). Cytosolic lipolysis and lipophagy: two sides of the same coin. Nat Rev Mol Cell Biol 18, 671-684.
Zhao, Y. G., Chen, Y., Miao, G., Zhao, H., Qu, W., Li, D., Wang, Z., Liu, N., Li, L., Chen, S., et al. (2017). The ER-Localized Transmembrane Protein EPG-3/VMP1 Regulates SERCA Activity to Control ER-Isolation Membrane Contacts for Autophagosome Formation. Mol Cell 67, 974-989 e976.
Zhao, Z., Thackray, L. B., Miller, B. C., Lynn, T. M., Becker, M. M., Ward, E., Mizushima, N. N., Denison, M. R., and Virgin, H.W.t. (2007). Coronavirus replication does not require the autophagy gene ATG5. Autophagy 3, 581-585.
Zhen, Y., Spangenberg, H., Munson, M. J., Brech, A., Schink, K. O., Tan, K. W., Sorensen, V., Wenzel, E. M., Radulovic, M., Engedal, N., et al. (2020). ESCRT-mediated phagophore sealing during mitophagy. Autophagy 16, 826-841.
Zhou, P., Yang, X. L., Wang, X. G., Hu, B., Zhang, L., Zhang, W., Si, H. R., Zhu, Y., Li, B., Huang, C. L., et al. (2020). A pneumonia outbreak associated with a new coronavirus of probable bat origin. Nature 579, 270-273.
Zhu, L., Mou, C., Yang, X., Lin, J., and Yang, Q. (2016). Mitophagy in TGEV infection counteracts oxidative stress and apoptosis. Oncotarget 7, 27122-27141.

1. A method of treating of treating an autophagy mediated disease state or condition in a patient or subject in need comprising administering to said patient an effective amount of a SIGMA 1 receptor modulator.

2. The method according to claim 1 wherein said SIGMA 1 receptor modulator is selected from the group consisting of haloperidol, BD-1047, cutamesine (SA4503), BD-1063, 4-IBP, CM-304, NE-100, S1RA (MR-309), FTC-146, AZ-66 or a mixture thereof.

3. The method according to claim 1 wherein said SIGMA 1 receptor modulator is selected from the group consisting of fluvoxamine, 4-PPBP (4-phenyl-1-(4-phenylbutylpiperidine)), haloperidol, BD-1047, cutamesine (SA4503), anavex2-73, PRE-084, Ditolylguanidine, dimethyltryptamine and siramesine.

4. The method according to claim 1 wherein said modulator is haloperidol, BD-1047, cutamesine, an isotopomer of BD-1047, an isotopomer of cutamesine or a mixture thereof.

5. The method according to claim 4 wherein said isotopomer of BD-1047 is a compound according to the chemical structure: 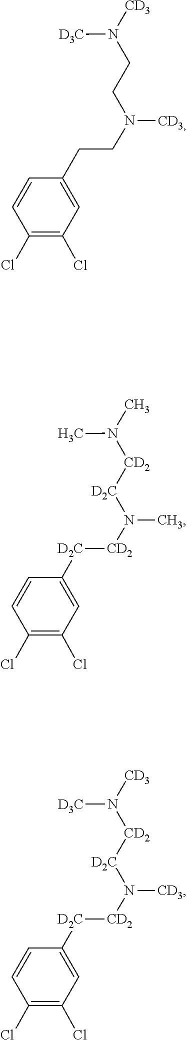
or a pharmaceutically acceptable salt or mixture thereof.

6. The method according to claim 4 wherein said isotopomer of cutamesine is a compound according to the chemical structure: 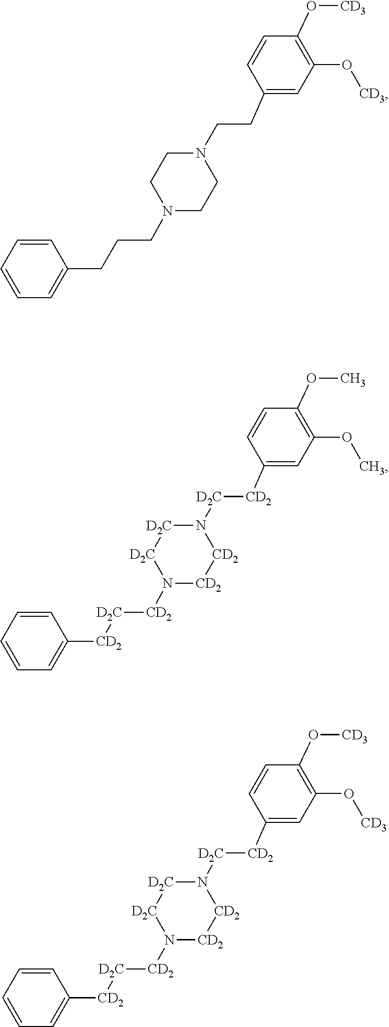
or a pharmaceutically acceptable salt thereof or mixture thereof.

7. The method according to claim 1 wherein said disease state or condition is a SARS-CoV, SARS-CoV-2 or MERS-CoV infection.

8. The method according to claim 1 wherein said SIGMA 1 receptor modulator is selected from the group consisting of haloperidol, BD-1047, cutamesine, BD-1063, 4-IBP, CM-304, NE-100, S1RA (MR-309), FTC-146, AZ-66 or a mixture thereof.

9. The method according to claim 7 wherein said modulator is haloperidol, BD-1047, isotopomeric BD-1047, cutamesine, isotopomeric cutamesine or a mixture thereof.

10. The method according to claim 9 wherein said modulator is isotopomeric BD-1047, isotopomeric cutamesine or a mixture thereof.

11. The method according to claim 1 wherein said modulator is administered in combination with bromhexine, ambroxol, chloroquine, hydroxychloroquine, azithromycin, ivermectin, zinc, molnupiravir, nirmatrelvir and ritonavir (Paxlovid), baricitinib, or a mixture thereof.

12. The method according to claim 1 wherein said modulator is further combined with an additional autophagy modulator.

13. The method according to claim 1 wherein said autophagy mediated disease state or condition is cancer, neurodegeneration, a lysosomal storage disease or a chronic inflammatory disease.

14. The method according to claim 1 wherein said autophagy mediated disease state or condition is inflammatory bowel disease, Crohn's disease, rheumatoid arthritis, lupus, multiple sclerosis, chronic obstructive pulmony disease/COPD, pulmonary fibrosis, cystic fibrosis, Sjogren's disease; a hyperglycemic disorder, diabetes (I or II), severe insulin resistance, hyperinsulinemia, insulin-resistant diabetes, Mendenhall's Syndrome, Werner Syndrome, leprechaunism, lipoatrophic diabetes, dyslipidemia, depressed high-density lipoprotein (HDL), elevated triglycerides, metabolic syndrome, liver disease including fibrosis, renal disease (apoptosis in plaques, glomerular disease), cardiovascular disease, including ischemia, stroke, pressure overload and complications during reperfusion, muscle degeneration and atrophy, symptoms of aging, frailty, low grade inflammation, atherosclerosis, stroke, age-associated dementia and sporadic forms of Alzheimer's disease, pre-cancerous states, psychiatric conditions including depression, stroke and spinal cord injury, arteriosclerosis, infectious diseases including microbial infections and embryogenesis/fertility/infertility.

15. The method according to claim 1 wherein said autophagy mediated disease state or condition is cancer, rheumatoid arthritis, malaria, antiphospholipid antibody syndrome, lupus, chronic urticaria or Sjogren's disease.

16. A pharmaceutical composition comprising an effective amount of haloperidol, cutamesine, an isotopomer of cutamesine, BD-1047, an isotopomer of BD-1047 or a mixture thereof in combination with bromhexine, ambroxol, chloroquine, hydroxychloroquine, azithromycin, ivermectin, zinc, molnupiravir, nirmatrelvir and ritonavir (Paxlovid), baricitinib, or a mixture thereof, further in combination with a pharmaceutically acceptable carrier, additive or excipient.

17. The composition according to claim 16 wherein said composition comprises an isotopomer of cutamesine, an isotopomer of BD-1047 or a mixture thereof in combination with bromhexine, ambroxol or a mixture thereof.

18. The composition according to claim 17 wherein said isotopomer of BD-1047 is a compound according to the chemical structure: 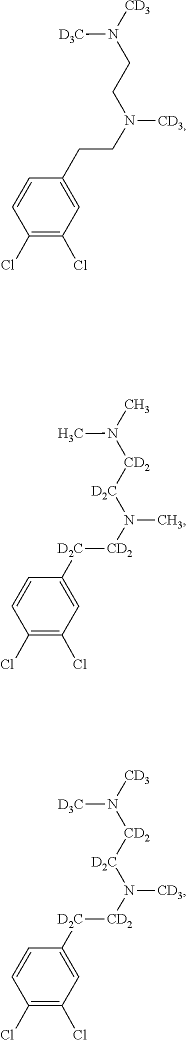
or a pharmaceutically acceptable salt or mixture thereof.

19. The composition according to claim 17 wherein said isotopomer of cutamesine is a compound according to the chemical structure: 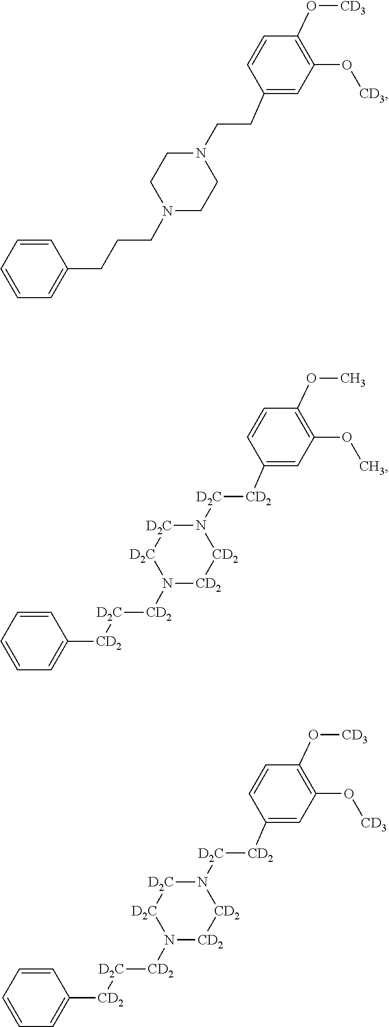
or a pharmaceutically acceptable salt thereof or mixture thereof.

20. The composition according to claim 16 which includes ambroxol.
